(19) United States
(12) Patent Application Publication
(10) Pub. No.: US 2013/0230459 A1
(43) Pub. Date: Sep. 5, 2013

(54) RADIOLABELLED mGluR2 PET LIGANDS

(71) Applicants: José Ignacio Andrés-Gil, Toledo (ES); Manuel Jesús Alcázar-Vaca, Toledo (ES); José Maria Cid-Núñez, Toledo (ES); Andrés Avelino Trabanco-Suárez, Toledo (ES); Guy Maurits R. Bormans, Leuven (BE); Sofie Jeanne Leopoldine Celen, Leuven (BE); Michel Koole, Leuven (BE)

(72) Inventors: José Ignacio Andrés-Gil, Toledo (ES); Manuel Jesús Alcázar-Vaca, Toledo (ES); José Maria Cid-Núñez, Toledo (ES); Andrés Avelino Trabanco-Suárez, Toledo (ES); Guy Maurits R. Bormans, Leuven (BE); Sofie Jeanne Leopoldine Celen, Leuven (BE); Michel Koole, Leuven (BE)

(73) Assignee: Janssen Pharmaceuticals, Inc., Titusville, NJ (US)

(21) Appl. No.: 13/884,073

(22) PCT Filed: Nov. 8, 2011
(86) PCT No.: PCT/EP2011/069643

§ 371 Date: May 8, 2013

(30) Foreign Application Priority Data
Nov. 8, 2010 (EP) 10190325.0

(51) Int. Cl. A61K 51/04; G01N 33/60; C07B 59/00; C07D 471/04

(52) U.S. Cl. CPC A61K 51/0455; C07D 471/04; G01N 33/60; C07B 59/002
USPC 424/1.81; 546/119; 435/7.21

(57) ABSTRACT

The present invention relates to novel, selective, radiolabelled mGluR2 ligands which are useful for imaging and quantifying the metabotropic glutamate receptor mGluR2 in tissues, using positron-emission tomography (PET). The invention is also directed to compositions comprising such compounds, to processes for preparing such compounds and compositions, to the use of such compounds and compositions for imaging a tissue, cells or a host, in vitro or in vivo and to precursors of said compounds.

### FIELD OF THE INVENTION

The present invention relates to novel, selective, radiolabelled mGluR2 ligands which are useful for imaging and quantifying the metabotropic glutamate receptor mGluR2 in tissues, using positron-emission tomography (PET). The invention is also directed to compositions comprising such compounds, to processes for preparing such compounds and compositions, to the use of such compounds and compositions for imaging a tissue, cells or a host, in vitro or in vivo and to precursors of said compounds.

### BACKGROUND OF THE INVENTION

Glutamate is the major amino acid neurotransmitter in the mammalian central nervous system. Glutamate plays a major role in numerous physiological functions, such as learning and memory but also sensory perception, development of synaptic plasticity, motor control, respiration, and regulation of cardiovascular function. Furthermore, glutamate is at the centre of several different neurological and psychiatric diseases, where there is an imbalance in glutamatergic neurotransmission.

Glutamate mediates synaptic neurotransmission through the activation of ionotropic glutamate receptor channels (iGluRs), and the NMDA, AMPA and kainate receptors which are responsible for fast excitatory transmission.

In addition, glutamate activates metabotropic glutamate receptors (mGluRs) which have a more modulatory role that contributes to the fine-tuning of synaptic efficacy.

Glutamate activates the mGluRs through binding to the large extracellular amino-terminal domain of the receptor, herein called the orthosteric binding site. This binding induces a conformational change in the receptor which results in the activation of the G-protein and intracellular signalling pathways. Eight different subtypes of mGluRs have been identified (mGluR1-8) which can be divided into three groups based on sequence homology, transduction mechanism and agonist pharmacology.

The mGluR2 subtype is negatively coupled to adenylate cyclase via activation of Gαi-protein, and its activation leads to inhibition of glutamate release in the synapse. In the central nervous system (CNS), mGluR2 receptors are abundant mainly throughout cortex, thalamic regions, accessory olfactory bulb, hippocampus, amygdala, caudate-putamen and nucleus accumbens.

Activating mGluR2 was shown in clinical trials to be efficacious to treat anxiety disorders. In addition, activating mGluR2 in various animal models was shown to be efficacious, thus representing a potential novel therapeutic approach for the treatment of schizophrenia, anxiety, depression, epilepsy, drug addiction/dependence, Parkinson's disease, pain, sleep disorders and Huntington's disease.

To date, most of the available pharmacological tools targeting mGluRs are orthosteric ligands which activate several members of the family as they are structural analogues of glutamate.

A new avenue for developing selective compounds acting at mGluRs is to identify compounds that act through allosteric mechanisms, modulating the receptor by binding to a site different from the highly conserved orthosteric binding site.

Positive allosteric modulators of mGluRs have emerged recently as novel pharmacological entities offering this attractive alternative. Various compounds have been described as mGluR2 positive allosteric modulators.

It was demonstrated that such compounds do not activate the receptor by themselves. Rather, they enable the receptor to produce a maximal response to a concentration of glutamate, which by itself induces a minimal response. Mutational analysis has demonstrated unequivocally that the binding of mGluR2 positive allosteric modulators does not occur at the orthosteric site, but instead at an allosteric site situated within the seven transmembrane region of the receptor.

Animal data suggest that positive allosteric modulators of mGluR2 have effects in anxiety and psychosis models similar to those obtained with orthosteric agonists. Allosteric modulators of mGluR2 were shown to be active in fear-potentiated startle, and in stress-induced hyperthermia models of anxiety. Furthermore, such compounds were shown to be active in reversal of ketamine- or amphetamine-induced hyperlocomotion, and in reversal of amphetamine-induced disruption of prepulse inhibition of the acoustic startle effect models of schizophrenia.

Recent animal studies further reveal that the selective positive allosteric modulator of metabotropic glutamate receptor subtype 2 biphenyl-indanone (BINA) blocks a hallucinogenic drug model of psychosis, supporting the strategy of targeting mGluR2 receptors for treating glutamatergic dysfunction in schizophrenia.

Positive allosteric modulators enable potentiation of the glutamate response, but they have also been shown to potentiate the response to orthosteric mGluR2 agonists such as LY379268 or DCG-IV. These data provide evidence for yet another novel therapeutic approach to treat the above mentioned neurological and psychiatric diseases involving mGluR2, which would use a combination of a positive allosteric modulator of mGluR2 together with an orthosteric agonist of mGluR2.

WO2010/130424, WO2010/130423 and WO2010/130422, published on 18 Nov. 2010, disclose mGluR2 positive allosteric modulators.

Our aim was to develop a positron emission tomography (PET) imaging agent to quantify the mGluR2 receptors in the brain. Positron Emission Tomography (PET) is a non-invasive imaging technique that offers the highest spatial and temporal resolution of all nuclear imaging techniques and has the added advantage that it can allow for true quantification of tracer concentrations in tissues. It uses positron emitting radionuclides such as, for example, ¹⁵O, ¹³N, ¹¹C and ¹⁸F for detection. Several positron emission tomography radiotracers have been reported so far for in vivo imaging of mGluR1 and mGluR5. Up to our knowledge there is not any PET ligand that has been disclosed for imaging mGluR2 so far.

### SUMMARY OF THE INVENTION

The present invention relates to a compound having the Formula (I)

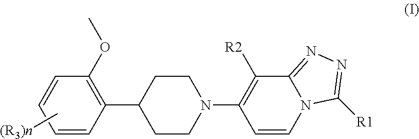

or a stereoisomeric form thereof, wherein

R¹ is selected from the group consisting of cyclopropylmethyl and C₁₋₃alkyl substituted with one or more fluoro substituents;

R² is selected from chloro and trifluoromethyl;

R³ is fluoro;

n is selected from 0, 1 and 2;

wherein at least one C is [¹¹C];

or a salt or a solvate thereof.

The invention also relates to precursor compounds for the synthesis of a compound of formula (I) as previously defined. Thus, the present invention also relates to a compound of formula (V)

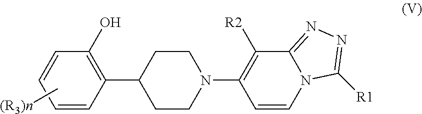

or a stereisomeric form thereof, wherein

R¹ is selected from the group consisting of cyclopropylmethyl and C₁₋₃alkyl substituted with one or more fluoro substituents;

R² is selected from chloro and trifluoromethyl;

R³ is fluoro;

n is selected from 0, 1 and 2;

or a salt or a solvate thereof;
with the proviso that 2-[1-[8-chloro-3-(cyclopropylmethyl)-1,2,4-triazolo[4,3-a]-pyridin-7-yl]-4-piperidinyl]-4-fluoro-phenol is excluded.

The invention also relates to reference materials, corresponding to the [¹²C]-compounds of formula (I). In an additional aspect, the invention relates to novel compounds selected from the group consisting of
8-chloro-3-(cyclopropylmethyl)-7-[4-(2,4-difluoro-6-methoxyphenyl)-1-piperidinyl]-1,2,4-triazolo[4,3-a]pyridine,
8-chloro-3-(cyclopropylmethyl)-7-[4-(3,6-difluoro-2-methoxyphenyl)-1-piperidinyl]-1,2,4-triazolo[4,3-a]pyridine,
8-chloro-3-(cyclopropylmethyl)-7-[4-(2,3-difluoro-6-methoxyphenyl)-1-piperidinyl]-1,2,4-triazolo[4,3-a]pyridine,
8-chloro-3-(cyclopropylmethyl)-7-[4-(3-fluoro-2-methoxyphenyl)-1-piperidinyl]-1,2,4-triazolo[4,3-a]pyridine,
8-chloro-3-(cyclopropylmethyl)-7-[4-(2-methoxyphenyl)-1-piperidinyl]-1,2,4-triazolo[4,3-a]pyridine,
8-chloro-3-(cyclopropylmethyl)-7-[4-(3,4-difluoro-2-methoxyphenyl)-1-piperidinyl]-1,2,4-triazolo[4,3-a]pyridine,
3-(cyclopropylmethyl)-7-[4-(3-fluoro-2-methoxyphenyl)-1-piperidinyl]-8-(trifluoromethyl)-1,2,4-triazolo[4,3-a]pyridine, and
3-(cyclopropylmethyl)-7-[4-(3,6-difluoro-2-methoxyphenyl)-1-piperidinyl]-8-(trifluoromethyl)-1,2,4-triazolo[4,3-a]pyridine;
   and the stereoisomeric forms, solvates and salts thereof.

Illustrative of the invention is a sterile solution comprising a compound of Formula (I) described herein.

Exemplifying the invention is a use of a compound of formula (I) as described herein, for, or a method of, imaging a tissue, cells or a host, in vitro or in vivo.

Further exemplifying the invention is a method of imaging a tissue, cells or a host, comprising contacting with or administering to a tissue, cells or a host, a compound of Formula (I) as described herein, and imaging the tissue, cells or host with a positron-emission tomography imaging system.

Additionally, the invention refers to a process for the preparation of a compound according to Formula (I) as described herein, wherein the C in the methoxy group is radiolabelled, herein referred to as [¹¹C]-(I), comprising the step of reacting a compound according to formula (V) as described herein, with [¹¹C]CH₃I or [¹¹C]CH₃OTf in the presence of a base in an inert solvent

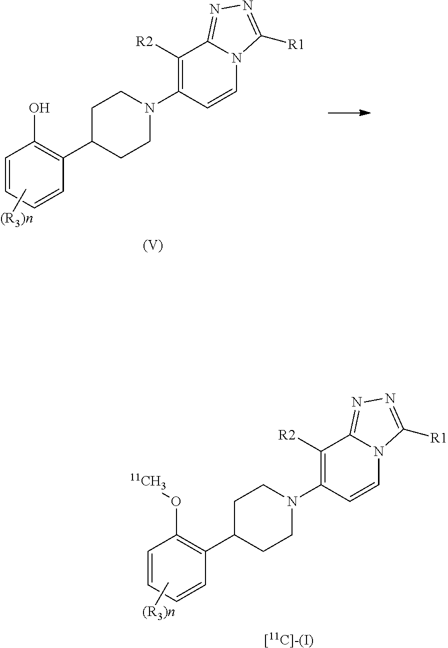

### DETAILED DESCRIPTION OF THE INVENTION

The present invention is directed to compounds of formula (I) as defined herein before, and pharmaceutically acceptable salts thereof. The present invention is also directed to precursor compounds of formula (V), used in the synthesis of compounds of formula (I).

In one embodiment of the present invention, R¹ is selected from cyclopropylmethyl and 2,2,2-trifluoroethyl; and R² is selected from chloro and trifluoromethyl.

In another embodiment of the present invention, R¹ is cyclopropylmethyl and R² is chloro.

In an additional embodiment of the present invention, n is 0 or 2.

In a further embodiment, the invention relates to a compound according to formula [¹¹C]-(I)

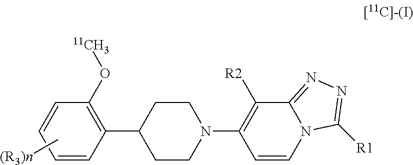

or a stereisomeric form thereof, wherein

R¹ is selected from the group consisting of cyclopropylmethyl and C₁₋₃alkyl substituted with one or more fluoro substituents;

R² is selected from chloro and trifluoromethyl;

R³ is fluoro;

n is selected from 0, 1 and 2;

or a salt or a solvate thereof.

In an additional embodiment, R¹ is selected from cyclopropylmethyl and 2,2,2-trifluoroethyl; and R² is selected from chloro and trifluoromethyl.

In another embodiment, R¹ is cyclopropylmethyl and R² is chloro.

In an additional embodiment, n is 0 or 2.

An additional embodiment of the invention relates to compounds wherein n is 2.

Compounds of formula (I) wherein n is 2 correspond to compounds wherein the phenyl ring is trisubstituted. In particular, such compounds, may be represented as (Ia) or (Ib) below

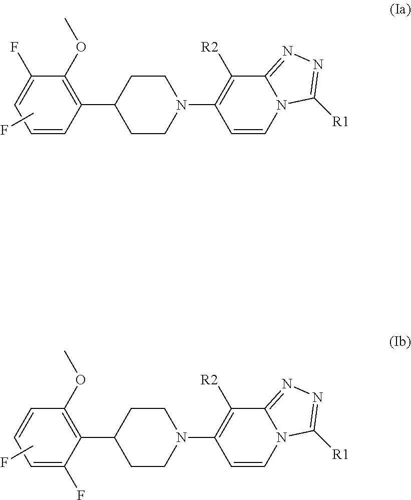

wherein R¹ and R² are as previously defined.

Compounds of formula [¹¹C]-(I) wherein n is 2 correspond to compounds wherein the phenyl ring is trisubstituted, in particular, such compounds, may be represented as [¹¹C]-(Ia) or [¹¹C]-(Ib) below

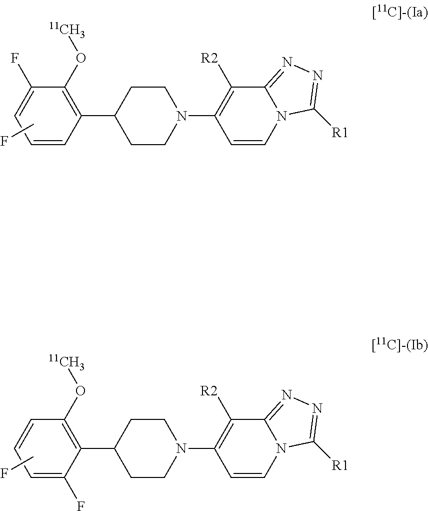

wherein R¹ and R² are as previously defined.

In a further embodiment, the compound of Formula (I) as previously described is selected from the group consisting of
8-chloro-3-(cyclopropylmethyl)-7-[4-[5-fluoro-2-[¹¹C]methoxyphenyl]-1-piperidinyl]-1,2,4-triazolo[4,3-a]pyridine,
8-chloro-3-(cyclopropylmethyl)-7-[4-[2-fluoro-6-[¹¹C]methoxyphenyl]-1-piperidinyl]-1,2,4-triazolo[4,3-a]pyridine,
8-chloro-7-[4-[5-fluoro-2-[¹¹C]methoxyphenyl]-1-piperidinyl]-3-(2,2,2-trifluoroethyl)-1,2,4-triazolo[4,3-a]pyridine,
8-chloro-7-[4-[2-fluoro-6-[¹¹C]methoxyphenyl]-1-piperidinyl]-3-(2,2,2-trifluoroethyl)-1,2,4-triazolo[4,3-a]pyridine,
8-chloro-3-(cyclopropylmethyl)-7-[4-[2,4-difluoro-6-[¹¹C]methoxyphenyl]-1-piperidinyl]-1,2,4-triazolo[4,3-a]pyridine,
8-chloro-3-(cyclopropylmethyl)-7-[4-(3,6-difluoro-2-[¹¹C]methoxyphenyl)-1-piperidinyl]-1,2,4-triazolo[4,3-a]pyridine,
8-chloro-3-(cyclopropylmethyl)-7-[4-[2,3-difluoro-6-[¹¹C]methoxyphenyl]-1-piperidinyl]-1,2,4-triazolo[4,3-a]pyridine,
8-chloro-3-(cyclopropylmethyl)-7-[4-[3-fluoro-2-[¹¹C]methoxyphenyl]-1-piperidinyl]-1,2,4-triazolo[4,3-a]pyridine,
8-chloro-3-(cyclopropylmethyl)-7-[4-[2-[¹¹C]methoxyphenyl]-1-piperidinyl]-1,2,4-triazolo[4,3-a]pyridine,
8-chloro-3-(cyclopropylmethyl)-7-[4-[3,4-difluoro-2-[¹¹C]methoxyphenyl]-1-piperidinyl]-1,2,4-triazolo[4,3-a]pyridine,
3-(cyclopropylmethyl)-7-[4-[3-fluoro-2-[¹¹C]methoxyphenyl]-1-piperidinyl]-8-(trifluoromethyl)-1,2,4-triazolo[4,3-a]pyridine, and
3-(cyclopropylmethyl)-7-[4-[3,6-difluoro-2-[¹¹C]methoxyphenyl]-1-piperidinyl]-8-(trifluoromethyl)-1,2,4-triazolo[4,3-a]pyridine;
   or a stereoisomeric form, or a salt or a solvate thereof.

In a further embodiment, the compound of Formula (V) as previously described is selected from the group consisting of
2-[1-[8-chloro-3-(cyclopropylmethyl)-1,2,4-triazolo[4,3-a]pyridin-7-yl]-4-piperidinyl]-3-fluoro-phenol,
2-[1-[8-chloro-3-(cyclopropylmethyl)-1,2,4-triazolo[4,3-a]pyridin-7-yl]-4-piperidinyl]-3,6-difluoro-phenol,
2-[1-[8-chloro-3-(cyclopropylmethyl)-1,2,4-triazolo[4,3-a]pyridin-7-yl]-4-piperidinyl]-3,5-difluoro-phenol,
2-[1-[8-chloro-3-(cyclopropylmethyl)-1,2,4-triazolo[4,3-a]pyridin-7-yl]-4-piperidinyl]-3,4-difluoro-phenol, and
2-[1-[3-(cyclopropylmethyl)-8-(trifluoromethyl)-1,2,4-triazolo[4,3-a]pyridin-7-yl]-4-piperidinyl]-3,6-difluoro-phenol;
   or a stereoisomeric form, or a salt or a solvate thereof.

As already mentioned, the compounds of Formula (I) and compositions comprising the compounds of Formula (I) can be used for imaging a tissue, cells or a host, in vitro or in vivo. In particular, the invention relates to a method of imaging or quantifying the mGluR2 receptor in a tissue, cells or a host in vitro or in vivo.

The cells and tissues are preferably central nervous system cells and tissues in which the mGluR2 receptors are abundant. As already mentioned, the mGluR2 receptor is abundant in central nervous system tissue, more in particular, in central nervous system tissue forming the brain; more in particular, forming the cerebral cortex, thalamic regions, accessory olfactory bulb, hippocampus, amygdala, caudate-putamen and nucleus accumbens.

When the method is performed in vivo, the host is a mammal. In such particular cases, the compound of Formula (I) is administered intravenously, for example, by injection with a syringe or by means of a peripheral intravenous line, such as a short catheter.

When the host is a human, the compound of Formula (I) or a sterile solution comprising a compound of Formula (I), may in particular be administered by intravenous administration in the arm, into any identifiable vein, in particular in the back of the hand, or in the median cubital vein at the elbow.

Thus, in a particular embodiment, the invention relates to a method of imaging a tissue or cells in a mammal, comprising the intravenous administration of a compound of Formula (I), as defined herein, or a composition comprising a compound of Formula (I) to the mammal, and imaging the tissue or cells with a positron-emission tomography imaging system.

Thus, in a further particular embodiment, the invention relates to a method of imaging a tissue or cells in a human, comprising the intravenous administration of a compound of Formula (I), as defined herein, or a sterile formulation comprising a compound of Formula (I) to the human, and imaging the tissue or cells with a positron-emission tomography imaging system.

In a further embodiment, the invention relates to a method of imaging or quantifying the mGluR2 receptor in a mammal, comprising the intravenous administration of a compound of Formula (I), or a composition comprising a compound of Formula (I) to the mammal, and imaging with a positron-emission tomography imaging system.

In another embodiment, the invention relates to the use of a compound of Formula (I) for imaging a tissue, cells or a host, in vitro or in vivo, or the invention relates to a compound of Formula (I), for use in imaging a tissue, cells or a host in vitro or in vivo, using positron-emission tomography.

### DEFINITIONS

“C₁₋₃alkyl” shall denote a straight or branched saturated alkyl group having 1, 2 or 3 carbon atoms, e.g. methyl, ethyl, 1-propyl and 2-propyl; “C₁₋₃alkyl substituted with one or more fluoro substituents” shall denote C₁₋₃alkyl as previously defined, substituted with 1, 2 or 3 or where possible, with more fluoro atoms.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the terms “compound of formula (I)”, “compound of formula [¹¹C]-(I)”, “compound of formula [¹¹C]-(Ia)”, “compound of formula [¹¹C]-(Ib)” and “compound of formula (V)” are meant to include the stereoisomers thereof. The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers. Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. Therefore, the invention includes enantiomers, diastereomers, racemates, and mixtures thereof. The absolute configuration may be specified according to the Cahn-Ingold-Prelog system. The configuration at an asymmetric atom may be specified by either R or S.

Addition salts of the compounds according to Formula (I) and of the compounds of Formula (V) can also form stereoisomeric forms and are also intended to be encompassed within the scope of this invention.

Acceptable salts of the compounds of formula (I) are those wherein the counterion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are included within the ambit of the present invention. The pharmaceutically acceptable salts are defined to comprise the therapeutically active non-toxic acid addition salt forms that the compounds according to Formula (I) are able to form. Said salts can be obtained by treating the base form of the compounds according to Formula (I) with appropriate acids, for example inorganic acids, for example hydrohalic acid, in particular hydrochloric acid, hydrobromic acid, sulphuric acid, nitric acid and phosphoric acid; organic acids, for example acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzensulfonic acid, p-toluenesulfonic acid, cyclamic acid, salicylic acid, p-aminosalicylic acid and pamoic acid.

Conversely, said salt forms can be converted into the free base form by treatment with an appropriate base.

In addition, some of the compounds of the present invention may form solvates with water (i.e., hydrates) or common organic solvents, and such solvates are also intended to be encompassed within the scope of this invention.

The term “host” refers to a mammal, in particular to humans, mice, dogs and rats.

The term “cell” refers to a cell expressing or incorporating the mGlu2 receptor.

The names of the compounds of the present invention were generated according to the nomenclature rules agreed upon by the Chemical Abstracts Service (CAS) using Advanced Chemical Development, Inc., software (ACD/Name product version 10.01; Build 15494, 1 Dec. 2006).

### Preparation

The compounds according to the invention can generally be prepared by a succession of steps, each of which is known to the skilled person. In particular, the compounds can be prepared according to the following synthesis methods.

### A. Preparation of the Final Compounds

Compounds of Formula (I) in their non-radiolabeled version, herein referred to as [¹²C]-(I) can be prepared by synthesis methods well known to the person skilled in the art. Compounds of the invention may be prepared, for example, by two different general methods:

### Method A:

Following the reaction sequence shown in scheme 1.

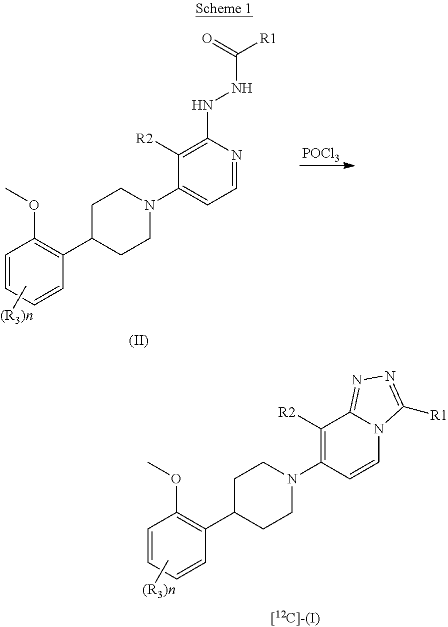

Thus, a final compound according to Formula [¹²C]-(I) wherein all variables are as previously defined, can be prepared following art known procedures by cyclization of an intermediate compound of Formula (II) in the presence of a halogenating agent such as for example POCl₃ in a suitable solvent such as, for example, CH₃CN or DCE, stirring the r.m. at a suitable temperature, using conventional heating or under microwave irradiation for the required time to achieve completion of the reaction, typically at 150-160° C. for 5-15 min in a microwave oven.

### Method B:

Alternatively, compounds of formula [¹²C]-(I) can also be prepared by a reaction sequence as shown in scheme 2, using different reaction conditions.

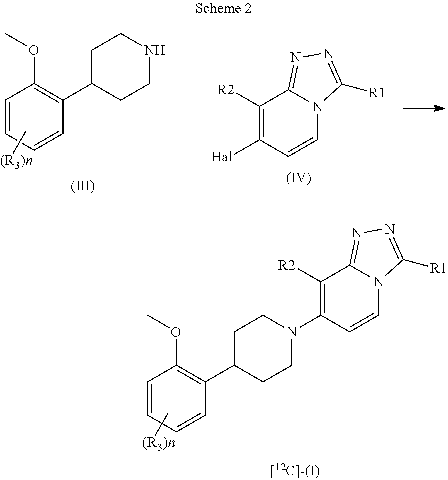

Thus, an intermediate compound of formula (III) can be reacted with an intermediate compound of formula (IV) in a suitable reaction-inert solvent such as, for example, toluene, in the presence of a suitable base such as, for example, Cs₂CO₃, a metal-based catalyst, specifically a palladium catalyst, such as palladium(II) acetate, and a suitable ligand, such as for example BINAP, heating for a suitable period of time that allows the completion of the reaction, typically at 100-125° C. overnight in a sealed tube. In reaction scheme (2) all variables are defined as in Formula (I) and halo is chloro, bromo or iodo, suitable for Pd-mediated coupling with amines

Alternatively, an intermediate compound (III) can be reacted with an intermediate compound (IV) in the presence of a base, such as for example DIPEA, NaHCO₃ or Cs₂CO₃, in a suitable inert solvent such as, for example, CH₃CN or propionitrile, stirring the r.m. at a suitable temperature, using conventional heating or under microwave irradiation for the required time to achieve completion of the reaction, typically at 190-230° C. for 15-30 min in a microwave oven, to yield a compound of Formula (I).

Compounds of formula (III) are either commercially available or can be prepared by standard synthetic procedures well known to the skilled person, some of which are further described.

### Radiolabelled Compounds:

The radiolabelling with radioactive carbon-11 of compounds of formula [¹²C]-(I) may be performed using radiochemical techniques well known to those skilled in the art, as shown in scheme 3.

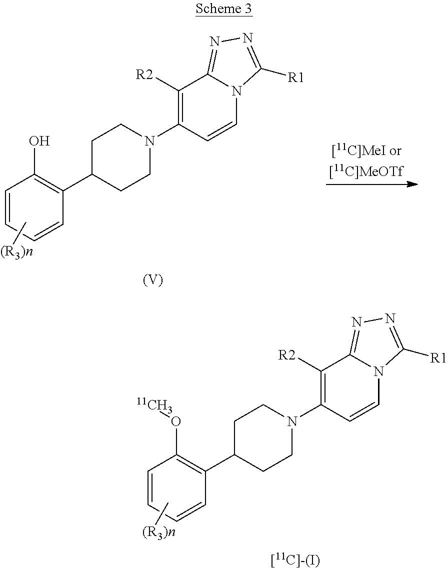

For example, a [¹¹C]-methoxy group can be incorporated by reaction of a suitable phenolic precursor of formula (V) with [¹¹C]CH₃I or [¹¹C]CH₃OTf in the presence of a base, such as for example Cs₂CO₃, in an inert solvent such as for example DMF, stirring the r.m. at a suitable temperature using conventional heating or under microwave irradiation, for a suitable period of time to allow completion of the reaction, typically with conventional heating at 90° C. for 3 min, followed by semi-preparative HPLC purification.

### B. Preparation of the Intermediate Compounds

Intermediate compounds according to Formula (II) can be prepared by art known procedures by reacting an intermediate of Formula (VI) with an acid halide of formula (VIIa), which is commercially available, as shown in scheme 4. The reaction can be carried out using an inert-solvent such as for example DCM in the presence of a base such as for example Et₃N, typically at r.t. for a suitable period of time to allow completion of the reaction. In reaction scheme 4 all variables are defined as in Formula (I).

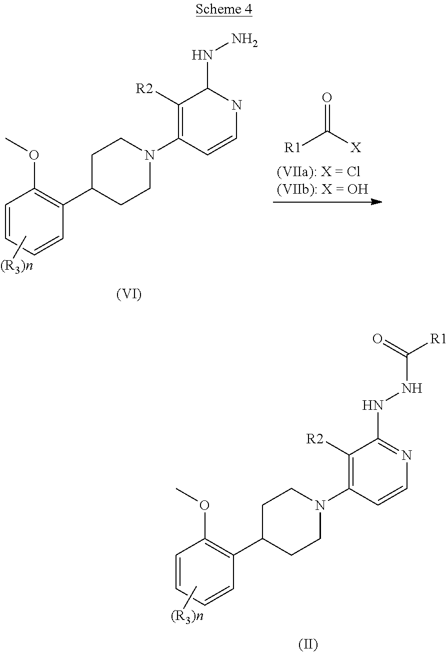

Alternatively, intermediate compounds according to Formula (II) can be prepared, following standard conditions that are known to those skilled in the art, by reacting an intermediate of Formula (VI) with a commercially available carboxylic acid of Formula (VIIb) via an amide bond formation reaction in the presence of a suitable coupling reagent.

Intermediate compounds according to Formula (VI) can be prepared by reacting an intermediate compound of Formula (VIII) with hydrazine-hydrate according to reaction scheme 5.

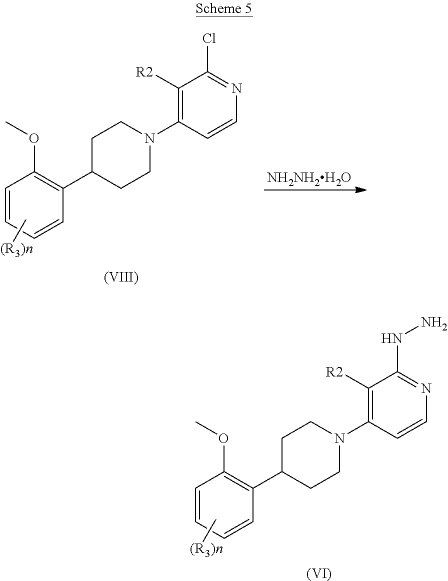

Thus, an intermediate compound (VIII) and hydrazine-hydrate are mixed in a suitable reaction-inert solvent, such as, for example, EtOH or THF and the mixture is stirred at a suitable temperature using conventional heating or under microwave irradiation, for a suitable period of time to allow completion of the reaction, typically at 160° C. under microwave irradiation for 20-40 min.

Intermediate compounds according to formula (VIII) can be prepared by a reaction sequence as shown in scheme 6.

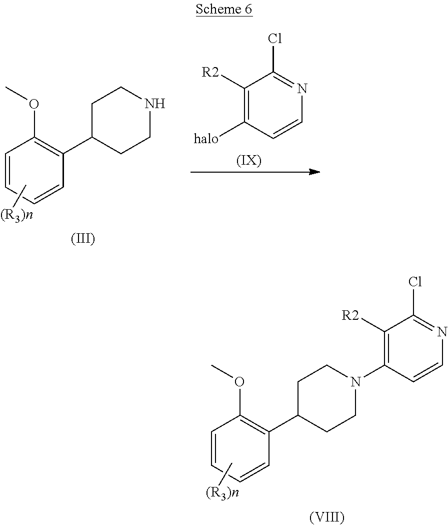

Therefore, an intermediate of Formula (III) can be reacted with an intermediate compound of Formula (IX) in a suitable reaction-inert solvent, such as, for example, CH₃CN, in the presence of a suitable base, such as, for example, DIPEA, heating the r.m. at a suitable temperature, using conventional heating or under microwave irradiation for the required time to achieve completion of the reaction, typically at 190° C. for 20 min in a microwave oven. In reaction scheme 6, halo is chloro, bromo or iodo.

Intermediate compounds of formula (IX) can be prepared by described synthesis methods well known to the person skilled in the art, such as, for example, by the reaction sequence shown in scheme 7 for intermediates wherein R² is chlorine, hereby named (IX-a).

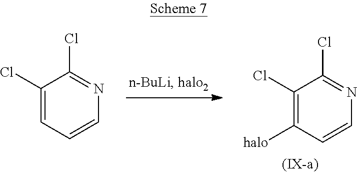

Thus, commercially available 23-dichloropyridine can be treated with an alkyl-lithium derivative, such as for example n-BuLi, in a suitable inert and dry solvent, such as for example Et₂O or THF, and reacted with the desired halogenating agent (halo₂), such as for example iodine, stirring the r.m. at a suitable temperature for the required time to achieve completion of the reaction, typically at −78° C. to r.t. overnight.

Intermediate compounds of Formula (IX) wherein R² is trifluoromethyl, hereby named (IX-b), can be prepared as shown in reaction scheme 8.

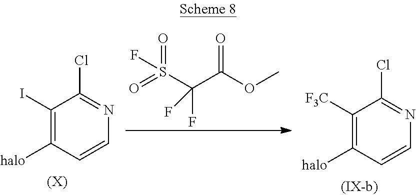

Thus, reaction of an intermediate of Formula (X) with a suitable trifluoromethylating agent, such as for example fluorosulfonyl(difluoro)acetic acid methyl ester, in a suitable reaction-inert solvent such as, for example, DMF in the presence of a suitable coupling agent such as for example, copper iodide, under thermal conditions such as, for example, heating the r.m. at 160° C. under microwave irradiation for 45 min, to afford intermediate of formula (IX-b).

Intermediate compounds of Formula (X) can be prepared as shown in scheme 9.

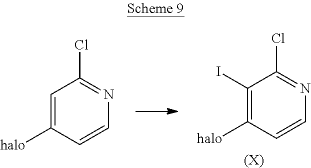

Therefore, a commercially available 2-chloro-4-halopyridine can be reacted with a strong base such as, for example, n-BuLi, and further treated with an iodinating agent such as, for example, iodine. This reaction is performed in a suitable reaction-inert solvent such as, for example, THF at low temperature for a period of time that allows the completion of the reaction, typically at −78° C. for 2 h.

Intermediate compounds of formula (III) can be prepared by a two step synthesis well known to the person skilled in the art, such as, for example, by the reaction sequence shown in scheme 10.

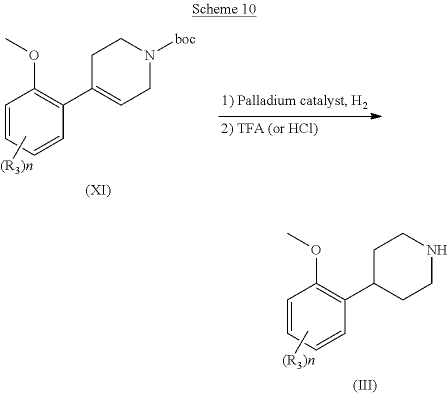

Therefore, a compound of formula (XI) can be subjected first to a hydrogenolysis reaction, in a suitable inert solvent in the presence of a catalyst such as, for example, 5% or 10% palladium on activated carbon, for a period of time that ensures the completion of the reaction, typically at 100° C. and 1 atmosphere of hydrogen in an H-cube apparatus. In a second step this intermediate can be deprotected with HCl in iPrOH or TFA in DCM, at a suitable temperature, typically r.t., for a period of time to allow cleavage of the BOC protecting group, typically 2 h. These two steps can be also reversed: first deprotection and then hydrogenation to give intermediate compound of formula (III). Intermediate compound of formula (III) wherein n=0 can be obtained from commercial sources.

Intermediate compounds according to formula (XI) can be prepared by synthesis methods well known to the person skilled in the art, such as, for example, by the reaction sequence shown in scheme 11.

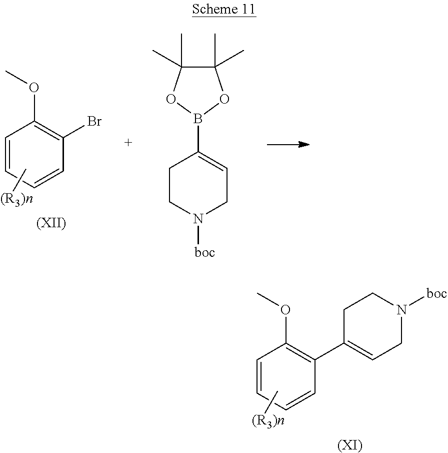

Thus, an intermediate compound of formula (XII) can be reacted with N-Boc-1,2,3,6-tetrahydropyridine-4-boronic acid pinacol ester, available from commercial sources, in the presence of a palladium(0) catalyst, such as, for example, Pd(PPh₃)₄, and in the presence of a base, such as, for example, K₂CO₃ or Cs₂CO₃, in a suitable inert solvent such as, for example, dioxane, stirring the r.m. at a suitable temperature using conventional heating or under microwave irradiation for the required time to achieve completion of the reaction, typically at 150° C. for 10 min in a microwave oven.

Intermediate compounds according to formula (XII) are either commercially available or can be prepared by synthesis methods well known by the skilled person, such as, for example, by the reaction sequence shown in scheme 12.

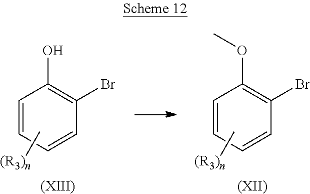

Therefore, an intermediate compound of formula (XIII) can be reacted with a methylating reagent, such as, for example, CH₃I, in the presence of a suitable base, such as, for example, K₂CO₃ or Cs₂CO₃, in a reaction-inert solvent, such as for example, CH₃CN, stirring the r.m. at a suitable temperature using conventional heating or under microwave irradiation for the required period of time to achieve completion of the reaction, typically at 150° C. for 10 min in a microwave oven.

Intermediate compounds according to formula (XIII) are either commercially available or can be prepared by synthesis methods well known to the skilled person, such as, for example, by the reaction sequence shown in scheme 13.

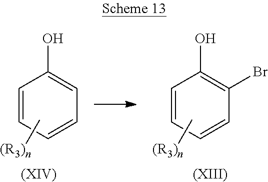

Thus, a phenolic intermediate of formula (XIV) can be brominated in ortho position to the hydroxyl with a brominating reagent, such as, for example, bromine or NBS, in the presence of an aliphatic amine, such as, for example, tert-butylamine, in a suitable inert solvent, such as, for example, DCM, stirring the r.m. at low temperature, typically at −10° C. or −40° C., for the required period of time to achieve completion of the reaction, typically 30 min.

Intermediate compounds according to formula (IV) can be prepared by a reaction sequence as shown in schemes 14 and 15.

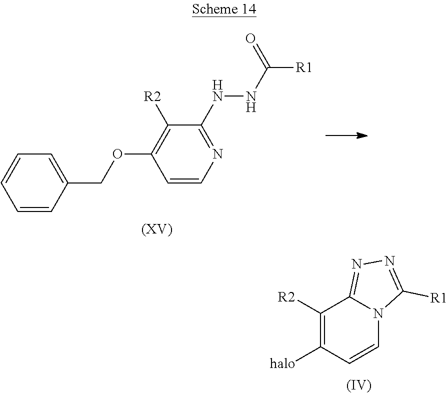

Thus, an intermediate compound of formula (IV) can be prepared following art known procedures by cyclization of an intermediate compound of Formula (XV) in the presence of an halogenating agent such as for example POCl₃ in a suitable solvent such as, for example, DCE, stirred under microwave irradiation, for a suitable period of time that allows the completion of the reaction, as for example 5 min at a temperature between 140-200° C.

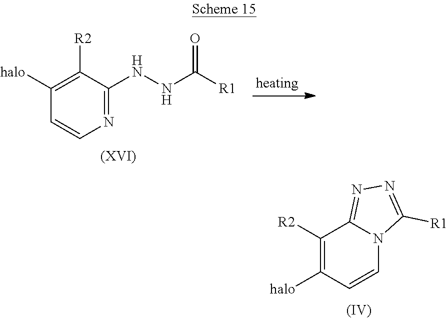

Alternatively, intermediate compounds of formula (IV) can be prepared following art known procedures, as shown in scheme 15, by cyclization of an intermediate compound of formula (XVI) after heating for a suitable period of time to allow the completion of the reaction, as for example 1 h at a temperature between 140-200° C. In reaction schemes 14 and 15 all variables are defined as in Formula (I) and halo is chloro, bromo or iodo.

Intermediate compounds according to Formula (XV) can be prepared by art known procedures such as, for example, by the reaction sequence shown in scheme 16.

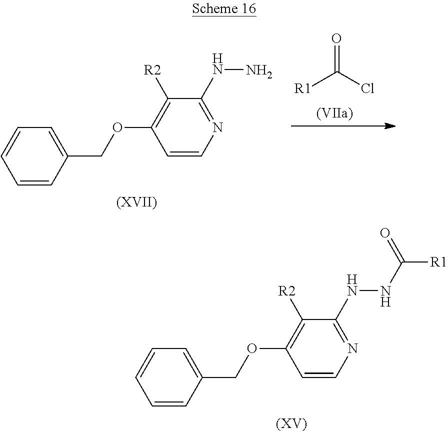

Thus, an intermediate compound of formula (XVII) can react with acid halides of formula (VIIa) in an inert-solvent, such as for example DCM, in the presence of a base such as for example Et₃N, usually at r.t. for a suitable period of time that allows completion of the reaction, for example 20 min, to yield an intermediate compound of formula (XV).

Intermediate compounds according to formula (XVI) can be prepared by art known procedures as shown in scheme 17.

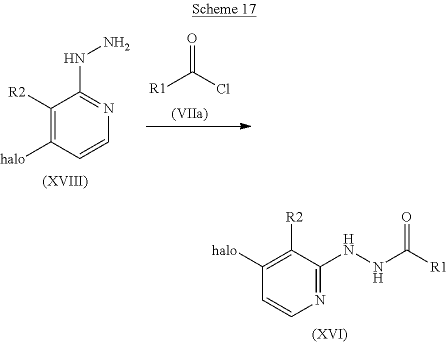

Thus, an intermediate of formula (XVI) can be prepared by reaction of intermediate compounds of formula (XVIII) with acid halides of formula (VIIa). The reaction can be carried out using an inert-solvent such as for example DCM in the presence of a base such as for example Et₃N, typically at r.t., for a suitable period of time that allows completion of the reaction, typically for 20 min.

Intermediate compounds according to Formula (XVIII) can be prepared by art known procedures such as, for example, by the reaction sequence shown in scheme 18.

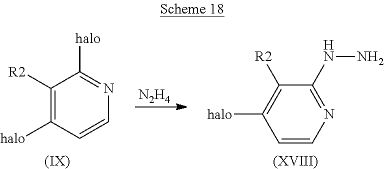

Thus, an intermediate compound of formula (IX) can be reacted with hydrazine in a suitable reaction-inert solvent, such as, for example, EtOH, THF or 1,4-dioxane at a suitable temperature using conventional heating or under microwave irradiation for the required period of time to achieve completion of the reaction, typically at 160° C. under microwave irradiation for 30 min, or by classical thermal heating at 70° C. overnight.

Intermediate compounds according to Formula (XVII) can be prepared by art known procedures such as, for example, by the reaction sequence shown in scheme 19.

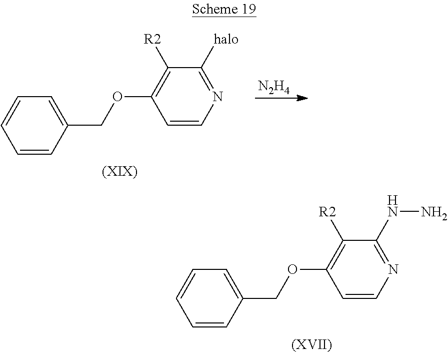

Thus, an intermediate compound of formula (XVII) can be prepared by reacting an intermediate compound of formula (XIX) with hydrazine in a suitable reaction-inert solvent, such as, for example, EtOH, THF or 1,4-dioxane at a suitable temperature using conventional heating or under microwave irradiation for the required period of time to achieve completion of the reaction, typically at 160° C. under microwave irradiation for 30 min, or by classical thermal heating at 70° C. overnight.

Intermediate compounds according to Formula (XIX) can be prepared as shown in scheme 20.

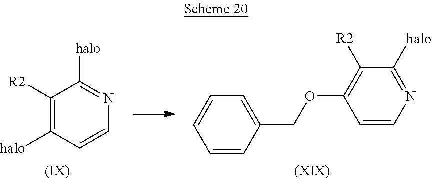

Thus, an intermediate compound of formula (IX) can be reacted with benzyl alcohol in a suitable reaction-inert solvent, such as, for example, DMF in the presence of a suitable base, such as for example NaH at r.t., for a suitable period of time that allows the completion of the reaction, typically for 1 h.

Intermediate compounds, precursors for the final radiolabelled compounds, according to Formula (V) can be prepared by several methods well known to the person skilled in the art. One of these methods is depicted in synthesis scheme 21.

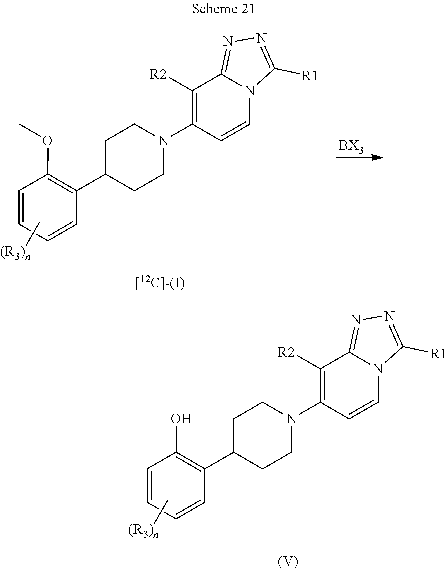

Thus, a final non-radiolabelled compound of formula (I), herein referred to as [¹²C]-(I) can be reacted with a Lewis acid such as, for example, BCl₃ or BBr₃, in a suitable inert solvent such as, for example, DCM, stirring the r.m. at a suitable temperature for the required time to achieve completion of the reaction, typically at r.t. for 30-45 min. Alternatively, intermediate compounds of formula (V) can also be synthesized by a reaction sequence as shown in scheme 22.

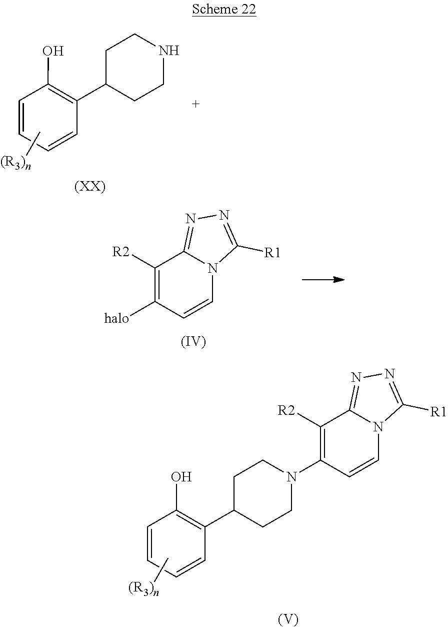

Therefore, an intermediate compound of formula (XX) can be reacted with an intermediate compound of formula (IV) in the presence of a suitable base, such as, for example, NaHCO₃, in an inert solvent such as, for example, CH₃CN, propionitrile or butyronitrile, stirring the r.m. at a suitable temperature, using conventional heating or under microwave irradiation for the required period of time to achieve completion of the reaction, typically at 180-230° C. for 10-30 min in a microwave oven, or for 1.5-16 h using conventional heating in a sealed tube.

Intermediate compounds according to Formula (XX) can be prepared by art known procedures such as, for example, by the reaction sequence shown in scheme 23.

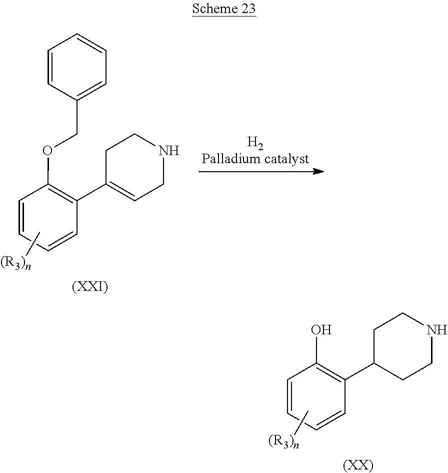

Thus, a compound of formula (XXI) can be subjected to a hydrogenolysis reaction, in a suitable inert solvent in the presence of a catalyst such as, for example, 5% or 10% palladium on activated carbon, for a period of time that ensures the completion of the reaction, typically at 100° C. and 1 atmosphere of hydrogen in an H-cube® apparatus. Intermediate compounds according to Formula (XXI) can be prepared by art known procedures such as, for example, by the reaction sequence shown in scheme 24.

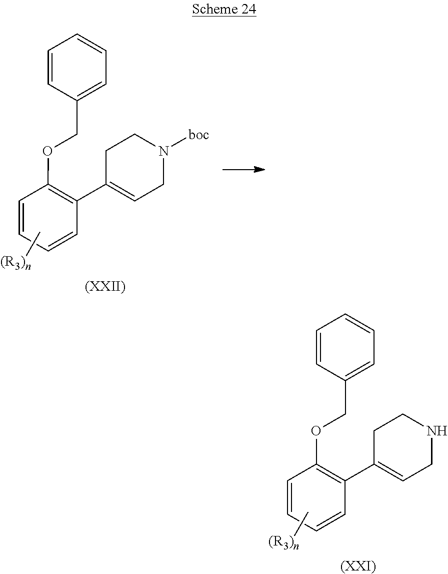

Thus, an intermediate compound according to formula (XXII) can be reacted with a diluted solution of an acid, such as, for example, HCl in iPrOH or TFA in DCM, at a suitable temperature, typically r.t., for a period of time to allow cleavage of the Boc protecting group, typically 2 h.

Intermediate compounds according to formula (XXII) can be prepared by synthesis methods well known to the person skilled in the art, such as, for example, by the reaction sequence shown in scheme 25.

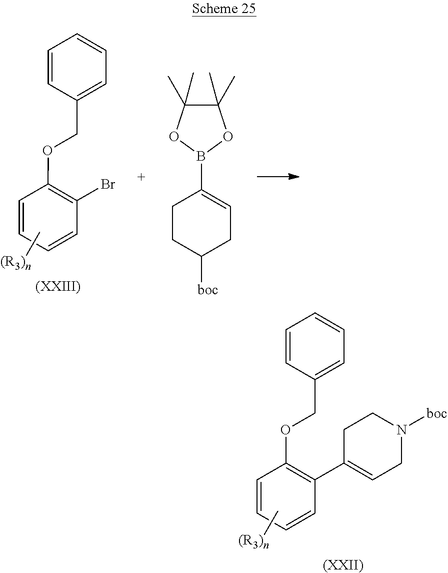

Therefore, an intermediate compound of formula (XXIII) can be reacted with N-Boc-1,2,3,6-tetrahydropyridine-4-boronic acid pinacol ester, available from commercial sources, in the presence of a palladium(0) catalyst, such as, for example, Pd(PPh₃)₄, and in the presence of a base, such as, for example, K₂CO₃ or Cs₂CO₃, in a suitable inert solvent such as, for example, dioxane, stirring the r.m. at a suitable temperature using conventional heating or under microwave irradiation for the required time to achieve completion of the reaction, typically at 150° C. for 10 min in a microwave oven. Intermediate compounds according to formula (XXIII) can be prepared by synthesis methods well known to the person skilled in the art, such as, for example, by the reaction sequence shown in scheme 26.

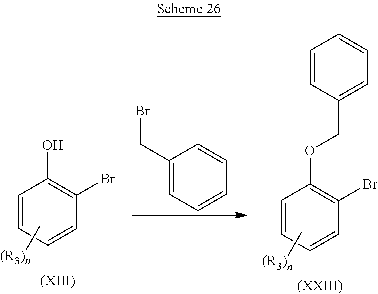

Thus, an intermediate compound of formula (XIII) can be reacted with benzyl bromide, in the presence of a suitable base such as, for example, K₂CO₃ or Cs₂CO₃, in an inert solvent such as, for example, CH₃CN, stirring the r.m. at a suitable temperature using conventional heating or under microwave irradiation for the required time to achieve completion of the reaction, typically at 150° C. for 10 min in a microwave oven.

### Applications

The compounds according to the present invention find various applications for imaging tissues, cells or a host, both in vitro and in vivo. Thus, for instance, they can be used to map the differential distribution of mGluR2 in subjects of different age and sex. Further, they allow one to explore for differential distribution of mGluR2 in subjects afflicted by different diseases or disorders. Thus, abnormal distribution may be helpful in diagnosis, case finding, stratification of subject populations, and in monitoring disease progression in individual subjects. The radioligands may further find utility in determining mGluR2 site occupancy by other ligands. Since the radioligand is administered in trace amounts, no therapeutic effect may be attributed to the administration of the radioligands according to the invention.

### EXPERIMENTAL PART

### I. Chemistry

As used herein, the term “LCMS” means liquid chromatography/mass spectrometry, “GCMS” means gas chromatography/mass spectrometry, “HPLC” means high-performance liquid chromatography, “aq.” means aqueous, “Boc”/“BOC” means tert-butoxycarbonyl, “nBuLi” means n-butyllithium, “DCE” means 1,2-dichloroethane, “DCM” means dichloromethane, “DMF” means N,N-dimethylformamide, “EtOH” means ethanol, “EtOAc” means ethyl acetate, “THF” means tetrahydrofuran, “DIPE” means diisopropyl ether, “DIPEA” means diisopropylethyl amine, “Et₃N” means triethylamine, “BINAP” means 1,1′-[1,1′-binaphthalene]-2,2′-diylbis[1,1-diphenyl-phosphine], “(±)BINAP” means Racemic-2-2′-bis(diphenylphosphino)-1,1′-binaphtyl, “min” means minutes, “h” means hours, “MeI” means methyl iodide, “NaOAc” means sodium acetate, “NBS” means N-bromosuccinimide, “iPrOH” means 2-propanol, “r.m.” means reaction mixture, “r.t.” means room temperature” “Rₜ” means retention time (in minutes), “Tf” means trifluoromethanesulfonate, “TFA” means trifluoroacetic acid, “quant.” means quantitative, “sat.” means saturated, “sol.” means solution, “[M+H]⁺” means the protonated mass of the free base of the compound, “[M−H]⁻” means the deprotonated mass of the free base of the compound, ‘m.p.” means melting point.

Microwave assisted reactions were performed in a single-mode reactor: Biotage Initiator™ Sixty microwave reactor (Biotage) or in a multimode reactor: MicroSYNTH Labstation (Milestone, Inc.).

Hydrogenation reactions were performed in a continuous flow hydrogenator H-CUBE® from ThalesNano Nanotechnology Inc.

Reactions under pressure were performed in a pressure tube (Q-Tube™) from Q-Labtech LLC.

Thin layer chromatography (TLC) was carried out on silica gel 60 F254 plates (Merck) using reagent grade solvents. Open column chromatography was performed on silica gel, mesh 230-400 particle size and 60 Å pore size (Merck) under standard techniques. Automated flash column chromatography was performed using ready-to-connect cartridges from Merck, on irregular silica gel, particle size 15-40 μm (normal phase disposable flash columns) on an SPOT or LAFLASH system from Armen Instrument.

Several methods for preparing the compounds of this invention are illustrated in the following examples, which are intended to illustrate but not to limit the scope of the present invention. Unless otherwise noted, all starting materials were obtained from commercial suppliers and used without further purification.

### A. Synthesis of Intermediates and Precursors

### Intermediate 1

### 2,3-Dichloro-4-iodo-pyridine (I-1)

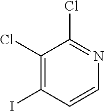

To a solution of n-BuLi (27.6 mL, 69 mmol, 2.5 M in hexanes) in dry Et₂O (150 mL) cooled at −78° C., under a nitrogen atmosphere, was added 2,2,6,6-tetramethylpiperidine (11.64 mL, 69 mmol) dropwise. The resulting r.m. was stirred at −78° C. for 10 min, and then a solution of 2,3-dichloropyridine (10 g, 67.57 mmol) in dry THF (75 mL) was added dropwise. The mixture was stirred at −78° C. for 30 min and then a solution of iodine (25.38 g, 100 mmol) in dry THF (75 mL) was added. The mixture was allowed to warm to r.t. overnight, quenched with Na₂S₂O₃ (aq sat. sol.) and extracted twice with EtOAc. The combined organic extracts were washed with NaHCO₃ (aq. sat. sol.), dried (Na₂SO₄) and concentrated in vacuo. The crude residue was precipitated with heptane, filtered off and dried to yield intermediate I-1 (8.21 g, 44%) as a pale cream solid.

### Intermediate 2

### (3-Chloro-4-iodo-pyridin-2-yl)hydrazine (I-2)

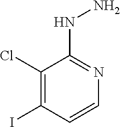

To a solution of intermediate I-1 (8 g, 29.21 mmol) in 1,4-dioxane (450 mL), was added hydrazine monohydrate (14.17 ml, 175.25 mmol). The r.m. was heated in a sealed tube at 70° C. for 16 h. After cooling, NH₄OH (32% aq. sol.) was added and the resulting mixture was concentrated in vacuo. The white solid residue thus obtained was taken up in EtOH. The suspension thus obtained was heated and then filtered off and the filtrate cooled to r.t. The precipitate formed was filtered off and then the filtrate concentrated in vacuo to yield intermediate compound I-2 (2.67 g, 52%) as a white solid.

### Intermediate 3

### N′-(3-chloro-4-iodo-pyridin-2-yl)-2-cyclopropylacetohydrazide (I-3)

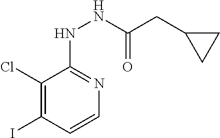 [SMILES: O=C(CC1CC1)NNc1nccc(I)c1Cl]

To a solution of intermediate I-2 (0.73 g, 2.71 mmol) in dry DCM (8 ml), cooled at 0° C., was added Et₃N (0.56 mL, 4.06 mmol) and cyclopropyl-acetyl chloride (0.38 g, 3.25 mmol). The resulting r.m. was stirred at r.t. for 16 h and then NaHCO₃ (aq. sat. sol.) was added. The resulting solution was extracted with DCM. The organic layer was separated, dried (MgSO₄) and concentrated in vacuo to yield intermediate I-3 (0.94 g, 99%).

### Intermediate 4

### 8-Chloro-3-cyclopropylmethyl-7-iodo[1,2,4]triazolo[4,3-a]pyridine (I-4)

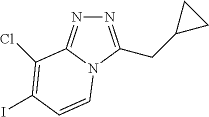 [SMILES: Clc1c(I)ccn2c(CC3CC3)nnc12]

Intermediate I-3 (0.74 g, 2.39 mmol) was heated at 160° C. for 40 min. After cooling, the brown gum thus obtained was triturated with DIPE yielding intermediate I-4 (0.74 g, 93%).

### Intermediate 5

### 2,4-Dichloro-3-iodo-pyridine (I-5)

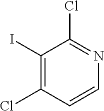

To a solution of 2,4-dichloropyridine (5.2 g, 35.14 mmol) and DIPEA (3.91 g, 38.65 mmol) in dry THF (40 mL) cooled at −78° C. under a nitrogen atmosphere, was added n-BuLi (24.16 mL, 38.65 mmol, 1.6 M in hexanes) dropwise. The resulting r.m. was stirred at −78° C. for 45 min and then a solution of iodine (9.81 g, 38.651 mmol) in dry THF (20 mL) was added dropwise. The mixture was stirred at −78° C. for 1 h, allowed to warm to r.t., diluted with EtOAc and quenched with NH₄Cl (aq. sat. sol.) and Na₂S₂O₃ (aq. sat. sol.). The organic layer was separated, washed with NaHCO₃ (aq. sat. sol.), dried (Na₂SO₄) and concentrated in vacuo. The crude product was purified by column chromatography (silica gel; Heptane/DCM up to 20% as eluent). The desired fractions were collected and concentrated in vacuo to yield intermediate I-5 (7.8 g, 81%).

### Intermediate 6

### 4-Dichloro-3-trifluoromethyl-pyridine (I-6)

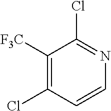 [SMILES: FC(F)(F)c1c(Cl)ccnc1Cl]

To a mixture of intermediate I-5 (2 g, 7.30 mmol) in DMF (50 mL) were added fluorosulfonyl-difluoro-acetic acid methyl ester [C.A.S. 680-15-9] (1.86 ml, 14.60 mmol) and copper (I) iodide (2.79 g, 14.60 mmol). The r.m. was heated in a sealed tube at 100° C. for 5 h. After cooling, the solvent was evaporated in vacuo. The crude product was purified by column chromatography (silica gel, DCM). The desired fractions were collected and concentrated in vacuo to yield intermediate I-6 (1.5 g, 95%).

### Intermediate 7

### 4-Benzyloxy-2-chloro-3-trifluoromethyl-pyridine (I-7)

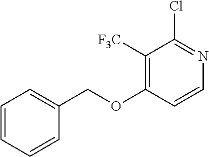 [SMILES: FC(F)(F)c1c(OCc2ccccc2)ccnc1Cl]

To a suspension of NaH (0.49 g, 12.73 mmol, 60% mineral oil) in DMF (50 mL) cooled at 0° C., was added benzyl alcohol (1.26 mL, 12.2 mmol). The resulting mixture was stirred for 2 min then; intermediate I-6 (2.5 g, 11.57 mmol) was added. The resulting r.m. was gradually warmed to r.t. and stirred for 1 h. The r.m. was quenched with water and extracted with Et₂O. The organic layer was separated, dried (Na₂SO₄) and concentrated in vacuo. The crude product was purified by column chromatography (silica; DCM in Heptane 0/100 to 100/0). The desired fractions were collected and concentrated in vacuo to yield intermediate I-7 (1.1 g, 33%).

### Intermediate 8

### 4-(benzyloxy)-2-hydrazino-3-(trifluoromethyl)pyridine (I-8)

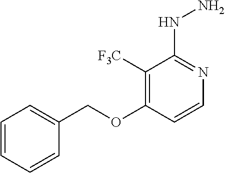 [SMILES: NNc1nccc(OCc2ccccc2)c1C(F)(F)F]

To a suspension of intermediate I-7 (1.09 g, 3.79 mmol) in 1,4-dioxane (9 mL), was added hydrazine monohydrate (3.67 mL, 75.78 mmol). The r.m. was heated at 160° C. under microwave irradiation for 30 min. After cooling, the resulting solution was concentrated in vacuo. The residue thus obtained was dissolved in DCM and washed with NaHCO₃ (aq. sat. sol.). The organic layer was separated, dried (Na₂SO₄) and concentrated in vacuo to yield intermediate I-8 (0.89 g, 83%) as a white solid.

### Intermediate 9

### N′-[4-(benzyloxy)-3-(trifluoromethyl)pyridin-2-yl]-2-cyclopropylacetohydrazide (I-9)

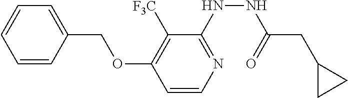 [SMILES: O=C(CC1CC1)NNc1nccc(OCc2ccccc2)c1C(F)(F)F]

To a solution of intermediate I-8 (0.89 g, 3.14 mmol) in dry DCM (3 mL) was added Et₃N (0.65 mL, 4.71 mmol) and cyclopropyl-acetyl chloride [C.A.S. 543222-65-5] (0.37 g, 3.14 mmol). The resulting r.m. was stirred at 0° C. for 20 min. The resulting mixture was then concentrated in vacuo to yield intermediate I-9 (1.1 g, 96%).

### Intermediate 10

### 7-Chloro-3-cyclopropylmethyl-8-trifluoromethyl[1,2,4]triazolo[4,3-a]pyridine (I-10)

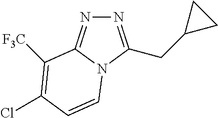 [SMILES: FC(F)(F)c1c(Cl)ccn2c(CC3CC3)nnc12]

Intermediate I-9 (1.14 g, 1.87 mmol) and POCl₃ (0.35 g, 3.74 mmol) in CH₃CN (10 mL) were heated at 150° C. under microwave irradiation for 10 min. After cooling, the resulting r.m. was diluted with DCM and washed with NaHCO₃ (aq. sat. sol.), dried (Na₂SO₄) and concentrated in vacuo. The crude product was purified by column chromatography (silica; 7M solution of NH₃ in MeOH in DCM 0/100 to 20/80). The desired fractions were collected and concentrated in vacuo to yield intermediate I-10 (0.261 g, 51%) as a white solid.

### Intermediate 11

### 2-Bromo-3,6-difluoro-phenol (I-11)

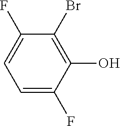

To a solution of 2,5-difluorophenol [C.A.S. 2713-31-7] (2.0 g, 15.37 mmol) and isopropylamine (1.61 ml, 15.37 mmol) in dry THF (40 mL) was added NBS (3.01 g, 16.19 mmol) portionwise at −40° C. The resulting r.m. was stirred at that temperature for 30 min and then allowed to get to r.t. The resulting mixture was diluted with HCl (1N in H₂O) and Et₂O, the organic layer was separated, dried (Na₂SO₄), and the solvent evaporated in vacuo to yield intermediate I-11 (3.23 g, 51% pure), that was used as such in the next reaction step.

### Intermediate 12

### 2-bromo-1,4-difluoro-3-methoxy-benzene (I-12)

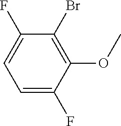

To a solution of intermediate I-11 (3.23 g, 15.45 mmol) in dry CH₃CN (25 mL), K₂CO₃ (6.4 g, 46.36 mmol) and MeI (2.88 mL, 46.36 mmol) were added, the resulting r.m. was heated under microwave irradiation at 150° C. for 10 min. Then the r.m. was diluted with DCM, filtered off and the filtrate solvent evaporated in vacuo to yield intermediate I-12 (3.45 g, 63% pure). The compound was used as such in the next reaction step.

### Intermediate 13

### 4-(3,6-Difluoro-2-methoxy-phenyl)-3,6-dihydro-2H-pyridine-1-carboxylic acid tert-butyl ester (I-13)

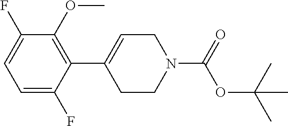 [SMILES: COc1c(F)ccc(F)c1C1=CCN(C(=O)OC(C)(C)C)CC1]

Intermediate I-12 (0.7 g, 3.14 mmol) was added to a stirred solution of 3,6-dihydro-4-(4,4,5,5-tetramethyl-1,3,2-dioxaborolan-2-yl)-1(2H)-pyridinecarboxylic acid, 1,1-dimethylethyl ester (1.26 g, 4.08 mmol) [C.A.S. 286961-14-6], Pd(PPh₃)₄ (0.07 g, 0.06 mmol) and K₂CO₃ (3.5 mL, aq. sat. sol.) in 1,4-dioxane (7 mL). The r.m. was heated under microwave irradiation at 150° C. for 10 min. After cooling, the mixture was diluted with water and extracted with Et₂O. The organic phase was separated, dried (Na₂SO₄) and the solvent evaporated in vacuo. The crude product was purified by column chromatography (silica gel; EtOAc in Heptane 10/90 to 20/80). The desired fractions were collected and concentrated in vacuo to give a residue that was triturated with Et₂O to yield intermediate I-13 (0.23 g, 22%).

### Intermediate 14

### 4-(3,6-Difluoro-2-methoxy-phenyl)-piperidine-1-carboxylic acid tert-butyl ester (I-14)

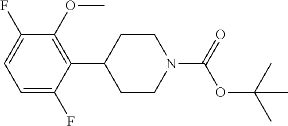 [SMILES: COc1c(F)ccc(F)c1C1CCN(C(=O)OC(C)(C)C)CC1]

A solution of intermediate I-13 (0.23 g, 0.71 mmol) in EtOH (15 mL) was hydrogenated in a H-Cube® reactor (1 ml/min, Pd(OH)₂ 20% cartridge, full H₂ mode, 80° C.). The solvent was evaporated in vacuo to yield intermediate I-14 (0.20 g, 84%).

### Intermediate 15

### 4-(3,6-Difluoro-2-methoxy-phenyl)-piperidine (I-15)

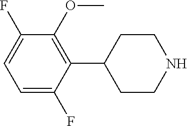 [SMILES: COc1c(F)ccc(F)c1C1CCNCC1]

Hydrochloric acid (7M in iPrOH) (2 mL) was added to a stirred solution of intermediate I-14 (0.20 g, 0.60 mmol) in MeOH (1 mL). The mixture was stirred at r.t. for 1.5 h. The mixture was diluted with Na₂CO₃ (aq. sat. sol.) and extracted with DCM. The organic phase was separated, dried (Na₂SO₄) and the solvent evaporated in vacuo to yield intermediate I-15 (0.12 g, 85%).

### Intermediate 16

### 4-(2-Fluoro-6-methoxy-phenyl)-3,6-dihydro-2H-pyridine-1-carboxylic acid tert-butyl easter (I-16)

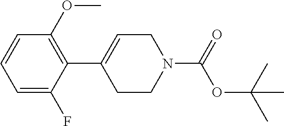 [SMILES: COc1cccc(F)c1C1=CCN(C(=O)OC(C)(C)C)CC1]

Intermediate I-16 was synthesized following the same methodology described for I-13: starting from 2-Bromo-3-fluoroanisole [C.A.S. 446-59-3] (3.18 g, 15.82 mmol) and 3,6-dihydro-4-(4,4,5,5-tetramethyl-1,3,2-dioxaborolan-2-yl)-1(2H)-pyridinecarboxylic acid, 1,1-dimethylethyl ester [C.A.S. 286961-14-6], (4 g, 12.9 mmol) to yield intermediate I-16 (6.63 g, quant. yield).

### Intermediate 17

### 4-(5-Fluoro-2-methoxy-phenyl)-3,6-dihydro-2H-pyridine-1-carboxylic acid tert-butyl ester (I-17)

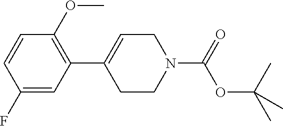 [SMILES: COc1ccc(F)cc1C1=CCN(C(=O)OC(C)(C)C)CC1]

Intermediate I-17 was synthesized following the same methodology described for I-13: starting from 2-Bromo-4-fluoroanisole [C.A.S. 452-08-4] (2.28 g, 11.12 mmol) and 3,6-dihydro-4-(4,4,5,5-tetramethyl-1,3,2-dioxaborolan-2-yl)-1(2H)-pyridinecarboxylic acid, 1,1-dimethylethyl ester (2.86 g, 9.26 mmol) [C.A.S. 286961-14-6], to yield intermediate I-17 (3.4 g, quant. yield).

### Intermediate 18

### 2-Bromo-1,5-difluoro-3-methoxyl-benzene (I-18)

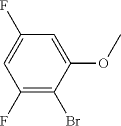

Intermediate I-18 was synthesised as reported for intermediate I-12. Starting from 2-Bromo-3,5-difluorophenol (0.5 g, 2.39 mmol) and MeI (0.22 mL, 3.58 mmol) to yield intermediate I-18 (0.53 g, quant. yield).

### Intermediate 19

### 4-(2,4-Difluoro-6-methoxy-phenyl)-3,6-dihydro-2H-pyridine-1-carboxylic acid tert-butyl ester (I-19)

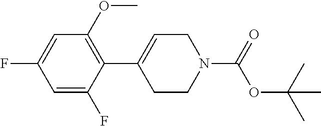 [SMILES: COc1cc(F)cc(F)c1C1=CCN(C(=O)OC(C)(C)C)CC1]

Intermediate I-19 was synthesized following the same methodology described for I-13: starting from intermediate I-18 (0.53 g, 2.39 mmol) and 3,6-dihydro-4-(4,4,5,5-tetramethyl-1,3,2-dioxaborolan-2-yl)-1(2H)-pyridinecarboxylic acid 1,1-dimethylethyl ester [C.A.S. 286961-14-6] (0.62 g, 1.99 mmol) to yield intermediate I-19 (1.2 g quant. yield).

### Intermediate 20

### 4-(2,3-Difluoro-6-methoxyl-phenyl)-3,6-dihydro-2H-pyridine-1-carboxylic acid tert-butyl ester (I-20)

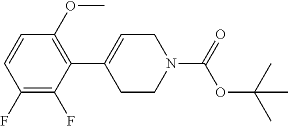 [SMILES: COc1ccc(F)c(F)c1C1=CCN(C(=O)OC(C)(C)C)CC1]

Intermediate I-20 was synthesized following the same synthetic pathway described for I-13: starting from 2-bromo-3,4-difluoroanisole [C.A.S. 935285-66-8] (0.79 g, 3.55 mmol) and 3,6-dihydro-4-(4,4,5,5-tetramethyl-1,3,2-dioxaborolan-2-yl)-1(2H)-pyridinecarboxylicacid 1,1-dimethylethyl ester (1 g, 3.23 mmol) [C.A.S. 286961-14-6], to yield intermediate I-20 (1.05 g, quant. yield).

### Intermediate 21

### 6-Bromo-2,3-difluoro-phenol (I-21)

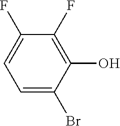

To a solution of 2,3-difluorophenol [C.A.S. 6418-38-8] (0.5 g, 3.84 mmol) and isopropylamine (0.40 ml, 3.84 mmol) in dry DCM (20 mL) was added NBS (3.01 g, 16.19 mmol) portionwise at −10° C. The resulting r.m. was stirred at that temperature for 30 min and then allowed to get to r.t. The resulting mixture was diluted with HCl (1N in H₂O) and the organic layer was separated, dried (Na₂SO₄), and the solvent evaporated in vacuo. The crude compound was purified by chromatography (silica gel, EtOAc in heptane 0:100 to 20:80). The desired fractions were collected the solvent evaporated in vacuo to yield intermediate I-21 (0.63 g, 78%).

### Intermediate I-22

### 1-Bromo-3,4-difluoro-2-methoxy-benzene (I-22)

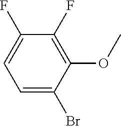

Intermediate I-22 was synthesized following the same methodology described for I-12: starting form intermediate I-21 (0.63 g, 3.01 mmol) treated with Met (0.28 mL, 4.51 mmol), derivative I-22 was afforded (0.62 g, 92.2%).

### Intermediate I-23

### 4-(3,4-Difluoro-2-methoxy-phenyl)-3,6-dihydro-2H-pyridine-1-carboxylic acid tert-butyl ester (I-23)

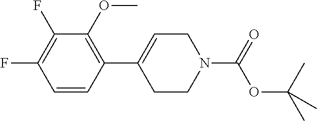 [SMILES: COc1c(C2=CCN(C(=O)OC(C)(C)C)CC2)ccc(F)c1F]

Intermediate I-23 was synthesized following the same methodology described for I-13: starting from intermediate I-22 (0.86 g, 3.83 mmol) treated with 3,6-dihydro-4-(4,4,5,5-tetramethyl-1,3,2-dioxaborolan-2-yl)-1(2H)-pyridinecarboxylic acid 1,1-dimethylethyl ester [C.A.S. 286961-14-6] (0.22 g, 0.19 mmol), intermediate I-23 was obtained (0.79 g, 63%).

### Intermediate I-24

### 4-(2-Fluoro-6-methoxy-phenyl)-1,2,3,6-tetrahydro-pyridine (I-24)

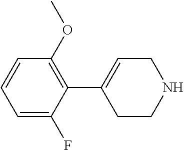

HCl (7M in iPrOH) (25 mL) was added to a stirred solution of intermediate I-16 (6.63 g, 0.60 mmol) in MeOH (15 mL). The mixture was stirred at r.t. for 1.5 h. The mixture was diluted with Na₂CO₃ (aq. sat. sol.) and extracted with DCM. The organic phase was separated, dried (Na₂SO₄) and concentrated in vacuo to yield intermediate I-24 (2 g, 74.5%).

### Intermediate I-25

### 4-(5-Fluoro-2-methoxy-phenyl)-1,2,3,6-tetrahydro-pyridine (I-25)

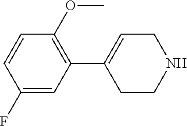

Intermediate I-25 was synthesized as reported for intermediate I-24: starting from intermediate I-17 (3.4 g, 7.41 mmol) and treated with HCl (7M in iPrOH) (23.5 mL), intermediate I-25 was obtained (1.7 g, quant. yield).

### Intermediate I-26

### 4-(2,4-Difluoro-6-methoxy-phenyl)-1,2,3,6-tetrahydro-pyridine (I-26)

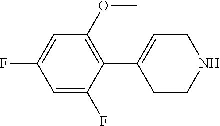 [SMILES: COc1cc(F)cc(F)c1C1=CCNCC1]

Intermediate I-26 was synthesized as reported for intermediate I-24: starting from intermediate I-19 (1.2 g, 1.99 mmol) and treated with HCl (7M in iPrOH) (4 mL), intermediate I-26 was obtained (0.33 g, 73.5%).

### Intermediate I-27

### 4-(2,3-Difluoro-6-methoxy-phenyl)-1,2,3,6-tetrahydro-pyridine (I-27)

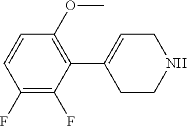 [SMILES: COc1ccc(F)c(F)c1C1=CCNCC1]

Intermediate I-27 was synthesized as reported for intermediate I-24: starting from intermediate I-20 (1.05 g, 3.23 mmol) and treated with HCl (7M in iPrOH) (10 mL), intermediate I-27 was obtained (0.34 g, 47.2%).

### Intermediate I-28

### 4-(3,4-Difluoro-2-methoxy-phenyl)-piperidine-1-carboxylic acid tert-butyl ester (I-28)

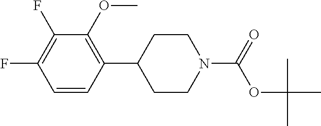 [SMILES: COc1c(C2CCN(C(=O)OC(C)(C)C)CC2)ccc(F)c1F]

Intermediate I-28 was synthesized as reported for intermediate I-14: starting from intermediate I-23 (0.54 g, 1.66 mmol) that was reduced to yield intermediate I-28 (0.54 g, quant. yield).

### Intermediate I-29

### 4-(2-Fluoro-6-methoxy-phenyl)-piperidine (I-29)

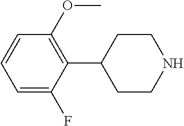

A solution of intermediate I-24 (2 g, 9.65 mmol) in EtOH (200 mL) was hydrogenated in a H-Cube® reactor (1.5 ml/min, Pd(OH)₂ 20% cartridge, full H₂ mode, 80° C.). The solvent was evaporated in vacuo to yield intermediate I-29 (1.8 g, 89.1%).

### Intermediate 30

### 4-(5-Fluoro-2-methoxy-phenyl)-piperidine (I-30)

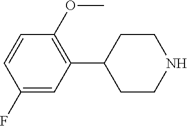

Intermediate I-30 was synthesized following the same methodology described for I-29: starting from intermediate I-25 that was reduced by hydrogenation to yield intermediate I-30 (0.76 g, 44.1%).

### Intermediate 31

### 4-(2,4-Difluoro-6-methoxy-phenyl)-piperidine (I-31)

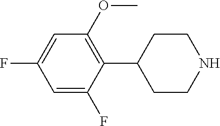 [SMILES: COc1cc(F)cc(F)c1C1CCNCC1]

Intermediate I-31 was synthesized following the same methodology described for I-29: starting from intermediate I-26 that was reduced by hydrogenation to yield intermediate I-31 (0.188 g, 71.6%).

### Intermediate 32

### 4-(2,3-Difluoro-6-methoxy-phenyl)-piperidine (I-32)

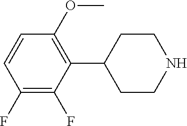 [SMILES: COc1ccc(F)c(F)c1C1CCNCC1]

Intermediate I-32 was synthesized following the same methodology described for I-29: starting from intermediate I-27 that was reduced by hydrogenation to yield intermediate I-32 (0.293 g, 84.4%).

### Intermediate 33

### 4-(3,4-Difluoro-2-methoxy-phenyl)-piperidine (I-33)

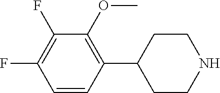 [SMILES: COc1c(C2CCNCC2)ccc(F)c1F]

Intermediate I-33 was synthesized following the same methodology described for I-15: upon treatment of I-28 with HCl (7 M in iPrOH) the N-boc protecting group was removed to yield I-33 (0.380 g, quant. yield).

### Intermediate 34

### 2-Benzyloxy-1-bromo-3-fluoro-benzene (I-34)

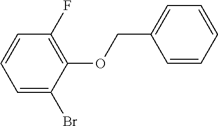 [SMILES: Fc1cccc(Br)c1OCc1ccccc1]

To a solution of 2-Bromo-6-fluorophenol [C.A.S. 2040-89-3] (1 g, 5.23 mmol) and benzylbromide [C.A.S. 100-39-0] (0.57 mL, 4.76 mmol) in CH₃CN (10 mL), K₂CO₃ (0.79 g, 5.71 mmol) was added. The r.m. was heated under microwave irradiation at 150° C. for 15 min. Then the r.m. was diluted with water and Et₂O, the organic layer separated, dried (Na₂SO₄), filtered and the solvent evaporated in vacuo. The residue was purified by column chromatography (silica gel, DCM in heptane 0/100 to 20/80) the desired fractions were collected and concentrated in vacuo to yield intermediate I-34 (1.34 g, quant. yield).

### Intermediate 35

### 4-(2-Benzyloxy-3-fluoro-phenyl)-3,6-dihydro-2H-pyridine-1-carboxylic acid tert-butyl ester (I-35)

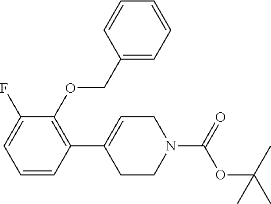 [SMILES: CC(C)(C)OC(=O)N1CC=C(c2cccc(F)c2OCc2ccccc2)CC1]

Intermediate I-34 (1.34 g, 4.76 mmol) was added to a stirred solution of 3,6-dihydro-4-(4,4,5,5-tetramethyl-1,3,2-dioxaborolan-2-yl)-1(2H)-pyridinecarboxylic acid, 1,1-dimethylethyl ester (1.23 g, 3.97 mmol) [C.A.S. 286961-14-6], Pd(PPh₃)₄ (0.14 g, 0.12 mmol) and K₂CO₃ (6 mL, aq. sat. sol.) in 1,4-dioxane (12 mL). The r.m. was heated under microwave irradiation at 150° C. for 10 min. After cooling, the mixture was diluted with water and extracted with EtOAc. The organic phase was separated, dried (Na₂SO₄), filtered and the solvent evaporated in vacuo. The crude product was purified by column chromatography, (silica gel, DCM in Heptane 50/50 to 100/0) the desired fractions were collected and concentrated in vacuo to yield intermediate I-35 (1.52, quant. yield).

### Intermediate I-36

### 4-(2-Benzyloxy-3-fluoro-phenyl)-1,2,3,6-tetrahydro-pyridine (I-36)

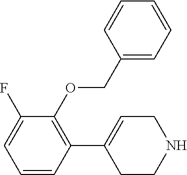 [SMILES: Fc1cccc(C2=CCNCC2)c1OCc1ccccc1]

HCl (7M in iPrOH) (15 mL) was added to a stirred solution of intermediate I-35 (1.52 g, 3.96 mmol) in MeOH (7.5 mL). The mixture was stirred at r.t. for 2 h. The mixture was diluted with water and extracted with Et₂O. The aqueous layer was separated and neutralized with Na₂CO₃ (aq. sat. sol.), then extracted with DCM, the organic layer was separated, dried (Na₂SO₄), filtered and the solvent evaporated in vacuo. The residue was purified by column chromatography (7M solution of NH₃ in MeOH in DCM 1/99 to 10/90) the desired fractions were collected, the solvent evaporated in vacuo to yield intermediate I-36 (0.78 g, 69.4%).

### Intermediate I-37

### 2-Fluoro-6-piperidin-4-yl-phenol (I-37)

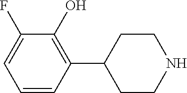 [SMILES: Oc1c(F)cccc1C1CCNCC1]

A solution of intermediate I-36 (0.78 g, 2.75 mmol) in EtOH (55 mL) was hydrogenated in an H-Cube® reactor (1 ml/min, Pd/C 10% cartridge, full H₂ mode, 100° C.). The solvent was evaporated in vacuo to yield intermediate I-37 (0.5 g, 93%).

### Intermediate I-38

### 1-Benzyloxy-2-bromo-4-fluoro-benzene (I-38)

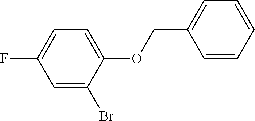 [SMILES: Fc1ccc(OCc2ccccc2)c(Br)c1]

Intermediate I-38 was synthesised following the same methodology described for I-34: starting from 2-Bromo-4-fluorophenol [C.A.S. 496-69-5] (1 g, 5.23 mmol) and benzyl bromide [C.A.S. 100-39-0] (0.62 mL, 5.23 mmol), intermediate I-38 was obtained (1.5 g, 98.5%).

### Intermediate I-39

### 1-Benzyloxy-2-bromo-3-fluoro-benzene (I-39)

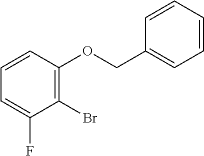 [SMILES: Fc1cccc(OCc2ccccc2)c1Br]

Intermediate I-39 was synthesised following the same methodology described for I-34: starting from 2-Bromo-3-fluorophenol [C.A.S. 443-81-2] (0.760 g, 3.97 mmol) and benzyl bromide [C.A.S. 100-39-0] (0.47 mL, 3.97 mmol) to yield intermediate I-39 (1.06 g, 94.7%).

### Intermediate I-40

### 2-Bromo-3,4,difluoro-phenol (I-40)

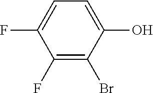

To a solution of 2-bromo-3-fluoroanisole [C.A.S. 935285-66-8] (1 g, 4.48) in DCM (2 mL), BBr₃ (17.93 mL, 17.93 mmol) was added dropwise at 0° C. The reaction was stirred 2 h at r.t. Then the excess of BBr₃ was quenched dropwise with water at 0° C., the organic layer was separated, dried (Na₂SO₄), filtered and the solvent evaporated in vacuo to yield intermediate I-40 (0.94 g, quant. yield) that was used as such in the next reaction step.

### Intermediate I-41

### 1-Benzyloxy-2-bromo-3,4-difluoro-benzene (I-41)

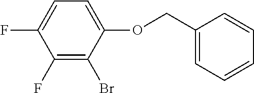 [SMILES: Fc1ccc(OCc2ccccc2)c(Br)c1F]

Intermediate I-41 was synthesised following the same methodology described for I-34: starting from intermediate I-40 (0.94 g, 4.49 mmol) and benzyl bromide [C.A.S. 100-39-0] (0.53 mL, 4.49 mmol) to yield intermediate I-41 (1.18 g, 88%).

### Intermediate I-42

### 1-Benzyloxy-2-bromo-3,5-difluoro-benzene (I-42)

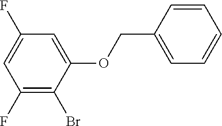 [SMILES: Fc1cc(F)c(Br)c(OCc2ccccc2)c1]

Intermediate I-42 was synthesised following the same methodology described for I-34: starting from 2-Bromo-3,5-difluorophenol [C.A.S. 325486-43-9] (1 g, 4.78 mmol) and benzyl bromide [C.A.S. 100-39-0] (0.569 mL, 4.78 mmol) to yield intermediate I-42 (1.43 g, quant. yield).

### Intermediate I-43

### 4-(2-Benzyloxy-5-fluoro-phenyl)-3,6-dihydro-2H-pyridine-1-carboxylic acid tert-butyl ester (I-43)

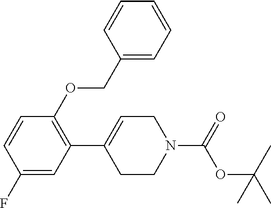 [SMILES: CC(C)(C)OC(=O)N1CC=C(c2cc(F)ccc2OCc2ccccc2)CC1]

Intermediate I-43 was synthesized as described for intermediate I-35. Starting from intermediate I-38 (1.48 g, 5.26 mmol) coupled with 3,6-dihydro-4-(4,4,5,5-tetramethyl-1,3,2-dioxaborolan-2-yl)-1(2H)-pyridinecarboxylic acid, 1,1-dimethylethyl ester [C.A.S. 286961-14-6] (1.36 g, 4.39 mmol) to yield intermediate I-43 (1.5 g, 85%).

### Intermediate I-44

### 4-(2-Benzyloxy-6-fluoro-phenyl)-3,6-dihydro-2H-pyridine-1-carboxylic acid tert-butyl ester (I-44)

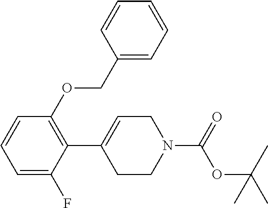 [SMILES: CC(C)(C)OC(=O)N1CC=C(c2c(F)cccc2OCc2ccccc2)CC1]

Intermediate I-44 was synthesized as described for intermediate I-35. Starting from intermediate I-39 (1.06 g, 3.77 mmol) coupled with 3,6-dihydro-4-(4,4,5,5-tetramethyl-1,3,2-dioxaborolan-2-yl)-1(2H)-pyridinecarboxylic acid, 1,1-dimethylethyl ester [C.A.S. 286961-14-6] (0.97 g, 3.14 mmol) to yield intermediate I-44 (1.01 g, 83.8%).

### Intermediate I-45

### 4-(6-Benzyloxy-2,3-difluoro-phenyl)-3,6-dihydro-2H-pyridine-1-carboxylic acid tert-butyl ester (I-45)

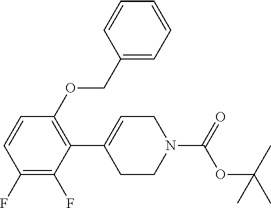 [SMILES: CC(C)(C)OC(=O)N1CC=C(c2c(OCc3ccccc3)ccc(F)c2F)CC1]

Intermediate I-45 was synthesized as described for intermediate I-35. Starting from intermediate I-41 (1.18 g, 3.96 mmol) coupled with 3,6-dihydro-4-(4,4,5,5-tetramethyl-1,3,2-dioxaborolan-2-yl)-1(2H)-pyridinecarboxylic acid, 1,1-dimethylethyl ester [C.A.S. 286961-14-6] (1.02 g, 3.3 mmol) to yield intermediate I-45 (0.9 g, 68%).

### Intermediate I-46

### 4-(2-Benzyloxy-4,6-difluoro-phenyl)-3,6-dihydro-2H-pyridine-1-carboxylic acid tert-butyl ester (I-46)

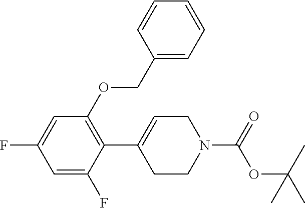 [SMILES: CC(C)(C)OC(=O)N1CC=C(c2c(F)cc(F)cc2OCc2ccccc2)CC1]

Intermediate I-46 was synthesized as described for intermediate I-35. Starting from intermediate I-42 (1.43 g, 4.78 mmol) coupled with 3,6-dihydro-4-(4,4,5,5-tetramethyl-1,3,2-dioxaborolan-2-yl)-1(2H)-pyridinecarboxylic acid, 1,1-dimethylethyl ester [C.A.S. 286961-14-6] (1.23 g, 3.98 mmol) to yield intermediate I-46 (1.51 g, 94.4%).

### Intermediate I-47

### 4-(2-Benzyloxy-5-fluoro-phenyl)-1,2,3,6-tetrahydro-pyridine (I-47)

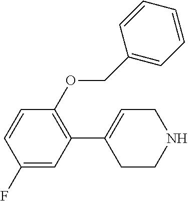 [SMILES: Fc1ccc(OCc2ccccc2)c(C2=CCNCC2)c1]

Intermediate I-47 was synthesized as described for intermediate I-36. Starting from I-43 (1.5 g, 3.91 mmol) and treated with HCl (7 M in iPrOH) (15 mL), intermediate I-47 was obtained (1.1 g, quant. yield).

### Intermediate I-48

### 4-(2-Benzyloxy-6-fluoro-phenyl)-1,2,3,6-tetrahydro-pyridine (I-48)

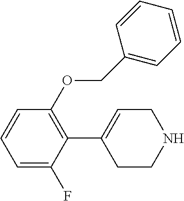 [SMILES: Fc1cccc(OCc2ccccc2)c1C1=CCNCC1]

Intermediate I-48 was synthesized as described for intermediate I-36. Starting from I-44 (1 g, 2.63 mmol) and treated with HCl (7 M in iPrOH) (5 mL), intermediate I-48 was obtained (0.46 g, 62%).

### Intermediate I-49

### 4-(6-Benzyloxy-2,3-difluoro-phenyl)-1,2,3,6-tetrahydro-pyridine (I-49)

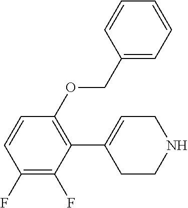 [SMILES: Fc1ccc(OCc2ccccc2)c(C2=CCNCC2)c1F]

Intermediate I-49 was synthesized as described for intermediate I-36. Starting from I-45 (0.9 g, 2.24 mmol) and treated with HCl (7 M in iPrOH) (5 mL), intermediate I-49 was obtained (0.38 g, 56.6%).

### Intermediate I-50

### 4-(2-Benzyloxy-4,6-difluoro-phenyl)-1,2,3,6-tetrahydro-pyridine (I-50)

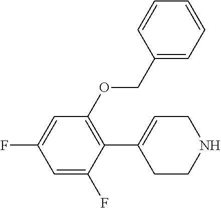 [SMILES: Fc1cc(F)c(C2=CCNCC2)c(OCc2ccccc2)c1]

Intermediate I-50 was synthesized as described for intermediate I-36. Starting from intermediate I-46 (1.51 g, 3.76 mmol) and treated with HCl (7 M in iPrOH) (7.5 mL), intermediate I-50 was obtained (1.07 g, 94%).

### Intermediate I-51

### 4-Fluoro-2-piperidine-4-yl-phenol (I-51)

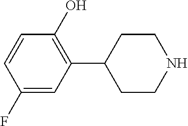 [SMILES: Oc1ccc(F)cc1C1CCNCC1]

Intermediate I-51 was synthesized following the same methodology described for I-37: Starting from intermediate I-47 (1.1 g, 3.88 mmol) through a hydrogenation, intermediate I-51 (0.75 g, 98%) was obtained.

### Intermediate I-52

### 3-Fluoro-2-piperidin-4-yl-phenol (I-52)

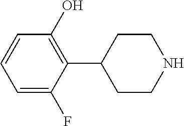 [SMILES: Oc1cccc(F)c1C1CCNCC1]

Intermediate I-52 was synthesized following the same methodology described for I-37: Starting from intermediate I-48 (0.46 g, 1.62 mmol) through a hydrogenation, intermediate I-52 (0.275 g, 86.5%) was obtained.

### Intermediate I-53

### 3,4-Difluoro-2-piperidin-4-yl-phenol (I-52)

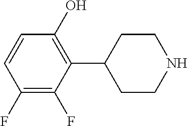 [SMILES: Oc1ccc(F)c(F)c1C1CCNCC1]

Intermediate I-53 was synthesized following the same methodology described for I-37: Starting from intermediate I-49 (0.38 g, 1.27 mmol) through a hydrogenation, intermediate I-53 (0.271 g, quant. yield) was obtained.

### Intermediate I-54

### 3,5-Difluoro-2-piperidine-4-yl-phenol (I-54)

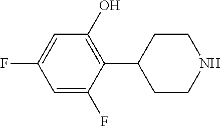 [SMILES: Oc1cc(F)cc(F)c1C1CCNCC1]

Intermediate I-54 was synthesized following the same methodology described for I-37: Starting from intermediate I-50 (1.07 g, 3.55 mmol) through a hydrogenation, intermediate I-54 (0.75 g, quant. yield) was obtained.

### Intermediate I-55

### 4-(3-Fluoro-2-hydroxy-phenyl)-piperidine-1-carboxylic acid tert-butyl ester (I-55)

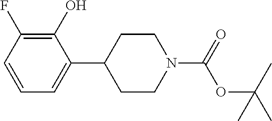 [SMILES: CC(C)(C)OC(=O)N1CCC(c2cccc(F)c2O)CC1]

To a solution of intermediate I-37 in DCM, di-tert-butyl-dicarbonate was added at 0° C., the r.m. was allowed to r.t. and stirred at this temperature for 30 min. Then HCl (2N in H₂O) was added, the organic layer was separated, dried (Na₂SO₄), filtered and the solvent evaporated in vacuo to yield intermediate I-55 (0.58 g, quant. yield), that was used as such in the next reaction step.

### Intermediate I-56

### 4-(3-Fluoro-2-methoxy-phenyl)-piperidine-1-carboxylic acid tert-butyl ester (I-56)

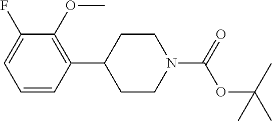 [SMILES: COc1c(F)cccc1C1CCN(C(=O)OC(C)(C)C)CC1]

Intermediate I-55 (0.58 g, 1.95 mmol), MeI (0.24 mL 3.9 mmol) and K₂CO₃ (0.54 g, 3.9 mmol) in CH₃CN (7.5 mL) were heated under microwave irradiation at 150° C. for 15 min. The mixture was diluted with H₂O and Et₂O. The organic layer was separated, dried (Na₂SO₄), filtered and the solvent evaporated in vacuo to yield intermediate I-56 (0.61 g, quant yield), that was used as such in the next reaction step.

### Intermediate I-57

### 4-(3-Fluoro-2-methoxy-phenyl)-piperidine (I-57)

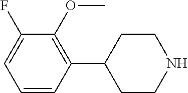

Intermediate I-57 was synthesized as described for I-29. Starting from intermediate I-56 (0.60 g, 1.95 mmol), after N-Boc deprotection, intermediate I-57 was obtained (0.29 g, 70.8%).

### Intermediate I-58

### 2-[1-[8-Chloro-3-(cyclopropylmethyl)-1,2,4-triazolo[4,3-a]pyridin-7-yl]-4-piperidinyl]-4-fluoro-phenol (I-58)

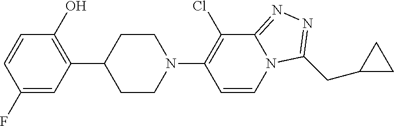 [SMILES: Oc1ccc(F)cc1C1CCN(c2ccn3c(CC4CC4)nnc3c2Cl)CC1]

To a mixture of intermediate I-4 (0.20 g, 0.61 mmol) and intermediate I-51 (0.18 g, 0.92 mmol) in propionitrile (1.5 mL), NaHCO₃ (0.15 g, 1.84 mmol) was added. The r.m. was heated under microwave irradiation at 230° C. for 30 min. Then the solvent was evaporated and the residue purified by column chromatography (silica gel, EtOAc in DCM 10/90 to 100/0), the desired fractions were collected and concentrated in vacuo, the compound obtained was then treated with EtOAc to yield intermediate I-58 (0.065 g, 26.38% yield). C₂₁H₂₂ClFN₄O. LCMS: Rt 3.04, m/z 401 [(M+H)]⁺ (method 1). ¹H NMR (400 MHz, DMSO-d₆) δ ppm 0.21-0.33 (m, 2H), 0.44-0.57 (m, 2H), 1.09-1.22 (m, 1H), 1.72-1.83 (m, 2H), 1.81-1.96 (m, 2H), 2.89-3.13 (m, 5H), 3.61 (br. d, J=11.8 Hz, 2H), 6.73-6.91 (m, 2H), 6.91-6.99 (m, 1H), 6.98 (d, J=7.6 Hz, 1H), 8.38 (d, J=7.4 Hz, 1H), 9.40 (s, 1H).

### Intermediate I-59

### 2-[1-[8-Chloro-3-(cyclopropylmethyl)-1,2,4-triazolo[4,3-a]pyridin-7-yl]-4-piperidinyl]-3-fluoro-phenol (I-59)

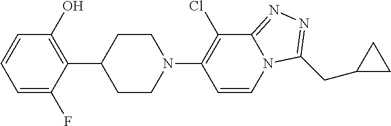 [SMILES: Oc1cccc(F)c1C1CCN(c2ccn3c(CC4CC4)nnc3c2Cl)CC1]

Intermediate I-59 was synthesized following the same synthetic procedure described for intermediate I-58. Starting from intermediate I-4 (0.1 g, 0.3 mmol) and I-52 (0.087 g, 0.45 mmol), derivative I-59 was obtained (0.034 g, 28.3%). C₂H₂₂ClFN₄O. LCMS: Rt 2.76, m/z 401 [(M+H)]⁺ (method 3). ¹H NMR (500 MHz, DMSO-d₆) δ ppm 0.21-0.34 (m, 2H), 0.45-0.56 (m, 2H), 1.05-1.21 (m, 1H), 1.67 (br. d, J=10.7 Hz, 2H), 2.25-2.35 (m, 2H), 2.97 (br. t, J=11.7 Hz, 2H), 3.02 (d, J=6.6 Hz, 2H), 3.22 (tt, J=12.3, 3.3 Hz, 1H), 3.60 (br. d, J=11.8 Hz, 2H), 6.56 (dd, J=10.4, 8.7 Hz, 1H), 6.67 (d, J=8.1 Hz, 1H), 6.97 (d, J=7.2 Hz, 1H), 6.99-7.07 (m, 1H), 8.39 (d, J=7.5 Hz, 1H), 9.96 (br. s., 1H).

### Intermediate I-60

### 2-[1-[8-Chloro-3-(cyclopropylmethyl)-1,2,4-triazolo[4,3-a]pyridin-7-yl)-4-piperidinyl]-3,4-difluoro-phenol (I-60)

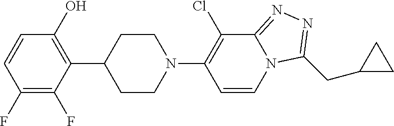 [SMILES: Oc1ccc(F)c(F)c1C1CCN(c2ccn3c(CC4CC4)nnc3c2Cl)CC1]

Intermediate I-60 was synthesized following the same synthetic procedure described for intermediate I-58. Starting from intermediates I-4 (0.1 g, 0.3 mmol) and I-53 (0.1 g, 0.45 mmol), intermediate I-60 was obtained (0.016 g, 11.6%). C₂₁H₂₁ClF₂N₄O. LCMS: Rt 2.85, m/z 419 [(M+H)]⁺ (method 3). ¹H NMR (500 MHz, DMSO-d₆) δ ppm 0.21-0.33 (m, 2H), 0.44-0.56 (m, 2H), 1.12-1.21 (m, 1H), 1.71 (br. d, J=10.7 Hz, 2H), 2.18-2.36 (m, 2H), 2.98 (br. t, J=11.7 Hz, 2H), 3.02 (d, J=6.6 Hz, 2H), 3.19-3.27 (m, 1H), 3.61 (br. d, J=11.8 Hz, 2H), 6.60 (dd, J=9.0, 2.9 Hz, 1H), 6.98 (d, J=7.5 Hz, 1H), 7.04 (q, J=9.5 Hz, 1H), 8.39 (d, J=7.5 Hz, 1H), 10.10 (br. s, 1H).

### Intermediate I-61

### 2-[1-[8-Chloro-3-(cyclopropylmethyl)-1,2,4-triazolo[4,3-a]pyridin-7-yl]-4-piperidinyl]-3,5-difluoro-phenol (I-61)

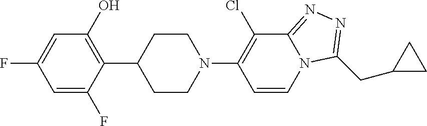 [SMILES: Oc1cc(F)cc(F)c1C1CCN(c2ccn3c(CC4CC4)nnc3c2Cl)CC1]

Intermediate I-61 was synthesized following the same synthetic procedure described for intermediate I-58. Starting from intermediate I-4 (0.1 g, 0.3 mmol) and I-54 (0.17 g, 0.6 mmol), intermediate I-61 was obtained (0.014 g, 11.2%). C₂₁H₂₁ClF₂N₄O. LCMS: Rt 2.97, m/z 419 [(M+H)]⁺ (method 3). ¹H NMR (500 MHz, DMSO-d₆) δ ppm 0.22-0.32 (m, 2H), 0.45-0.56 (m, 2H), 1.12-1.21 (m, 1H), 1.66 (br. d, J=10.7 Hz, 2H), 2.18-2.34 (m, 2H), 2.96 (br. t, J=11.7 Hz, 2H), 3.02 (d, J=6.9 Hz, 2H), 3.10-3.20 (m, 1H), 3.59 (br. d, J=11.8 Hz, 2H), 6.48 (br. d, J=10.4 Hz, 1H), 6.51-6.61 (m, 1H), 6.96 (d, J=7.5 Hz, 1H), 8.37 (d, J=7.5 Hz, 1H), 10.44 (br. s., 1H).

### Intermediate I-62

### 2-[1-[8-Chloro-3-(cyclopropylmethyl)-1,2,4-triazolo[4,3-a]pyridin-7-yl]-4-piperidinyl]-3,6-difluoro-phenol (I-62)

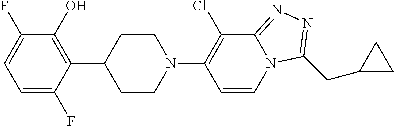 [SMILES: Oc1c(F)ccc(F)c1C1CCN(c2ccn3c(CC4CC4)nnc3c2Cl)CC1]

To a solution of compound B-2 (0.05 g, 0.116) in DCM (0.5 mL), BBr₃ (0.231 mL, 0.231 mmol) was added dropwise at 0° C. The reaction was stirred 45 min at r.t. The excess of BBr₃ was quenched dropwise with 1 mL of MeOH at 0° C. and then Na₂CO₃ (sat. aq. sol.) was added (to pH˜7). The organic layer was separated, dried (Na₂SO₄), filtered and the solvent evaporated in vacuo. The residue was purified by column chromatography (silica gel, MeOH in DCM 0/100 to 6/94), the desired fractions were collected and the solvent evaporated in vacuo. The compound obtained was then treated with CH₃CN and then purified again by chromatography (same eluent as before), and then treated with Et₂O to yield finally intermediate I-62 (0.018 g, 38%). C₂₁H₂₁ClF₂N₄O. LCMS: Rt 2.02, m/z 419 [(M+H)]⁺ (method 4). ¹H NMR (500 MHz, DMSO-d₆) δ ppm 0.21-0.35 (m, 2H), 0.45-0.56 (m, 2H), 1.11-1.22 (m, 1H), 1.70 (br. d, J=10.7 Hz, 2H), 2.24-2.40 (m, 2H), 2.98 (br. t, J=11.8 Hz, 2H), 3.02 (d, J=6.9 Hz, 2H), 3.24 (tt, J=12.4, 3.3 Hz, 1H), 3.61 (br. d, J=11.8 Hz, 2H), 6.63 (td, J=9.8, 3.9 Hz, 1H), 6.97 (d, J=7.5 Hz, 1H), 7.07 (td, J=9.7, 4.9 Hz, 1H), 8.38 (d, J=7.2 Hz, 1H), 9.99 (br. s., 1H).

### Intermediate I-63

### 2-[1-[3-(Cyclopropylmethyl)-8-(trifluoromethyl)-1,2,4-triazolo[4,3-a]pyridin-7-yl]-4-piperidinyl]-3,6-difluoro-phenol (I-63)

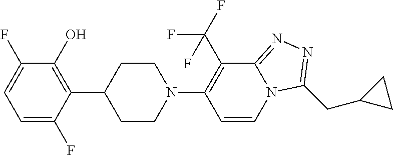 [SMILES: Oc1c(F)ccc(F)c1C1CCN(c2ccn3c(CC4CC4)nnc3c2C(F)(F)F)CC1]

Intermediate I-63 was synthesised following the same approach reported for I-62.

Starting from compound B-3 (0.15 g, 0.32 mmol) after deprotection with BBr₃, intermediate I-63 was obtained (0.01 g, 8.9%). C₂₂H₂₁F₅N₄O. LCMS: Rt 2.92, m/z 453 [(M+H)]⁺ (method 3). ¹H NMR (500 MHz, DMSO-d₆) δ ppm 0.21-0.35 (m, 2H), 0.42-0.59 (m, 2H), 1.11-1.21 (m, 1H), 1.67 (br. d, J=11.0 Hz, 2H), 2.15-2.34 (m, 2H), 3.00 (d, J=6.9 Hz, 2H), 3.17 (br. t, J=12.1 Hz, 2H), 3.53 (br. d, J=12.4 Hz, 2H), 6.60 (td, J=9.5, 3.3 Hz, 1H), 7.00 (d, J=7.8 Hz, 1H), 7.05 (td, J=9.6, 5.1 Hz, 1H), 8.47 (d, J=7.5 Hz, 1H), 9.96 (br. s., 1H).

### Intermediate I-64

### 2′,3′-Dichloro-4-(5-fluoro-2-methoxy-phenyl)-3,4,5,6-tetrahydro-2H-[1,4′]bipyridinyl (I-64)

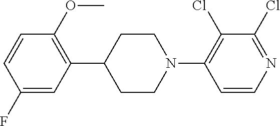 [SMILES: COc1ccc(F)cc1C1CCN(c2ccnc(Cl)c2Cl)CC1]

To a suspension of intermediates I-30 (0.79 g, 3.78 mmol) and I-1 (0.87 g, 3.15 mmol) in CH₃CN (8 mL), DIPEA (1.37 mL, 7.89 mmol) was added. The r.m. was heated at 110° C. overnight. Then the solvent was evaporated and the crude mixture was purified by column chromatography (silica gel, DCM in heptane 80/20), the desired fractions were collected, and concentrated in vacuo to yield intermediate I-64 (0.55 g, 48.5%).

### Intermediate I-65

### [3′-Chloro-4-(5-fluoro-2-methoxy-phenyl)-3,4,5,6-tetrahydro-2H-[1,4′]bipyridinyl-2′-yl]-hydrazine (I-65)

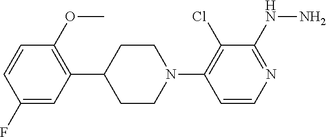 [SMILES: COc1ccc(F)cc1C1CCN(c2ccnc(NN)c2Cl)CC1]

To a suspension of intermediate I-64 (0.55 g, 1.53 mmol) in EtOH, hydrazine hydrate (50-60% in H₂O, 1.52 mL, 30.68 mmol) was added. The r.m. was heated under microwave irradiation at 160° C. for 20 min. After that more hydrazine hydrate (1.52 mL) was added and the mixture was irradiated again at the same temperature as before for 25 min. The solvent was then evaporated in vacuo to yield intermediate I-65 (0.5 g, 92.8%) that was used as such in the next reaction step.

### Intermediate I-66

### 3,3,3-Trifluoro-propionic acid N′-[3′-chloro-4-(5-fluoro-2-methoxy-phenyl)-3,4,5,6,tetrahydro-2H-[1,4′]bipyridinyl-2′-yl]-hydrazide (I-66)

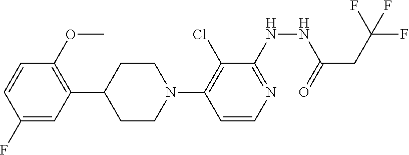 [SMILES: COc1ccc(F)cc1C1CCN(c2ccnc(NNC(=O)CC(F)(F)F)c2Cl)CC1]

To a solution of intermediate I-65 (0.53 g, 3.51 mmol) in dry DCM (10 ml) cooled at 0° C. was added Et₃N (0.52 mL, 3.78 mmol) and 3,3,3-trifluoropropionyl chloride [C.A.S. 41463-83-6] (0.29 mg, 1.96 mmol). The resulting r.m. was gradually warmed to r.t. and stirred for 1 h. Then more 3,3,3-trifluoropropionyl chloride was added and the mixture was stirred at r.t. overnight. The r.m. was washed with NaHCO₃ (sat. aq. sol.) and extracted with DCM. The organic phase was separated, dried (Na₂SO₄), and concentrated in vacuo to yield intermediate I-66 (0.35 g, 54.8%) that was used as such in the next reaction step.

### Intermediate I-67

### 2′,3′-Dichloro-4-(2-fluoro-6-methoxy-phenyl)-3,4,5,6-tetrahydro-2H-[1,4′]bipyridinyl (I-67)

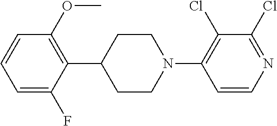 [SMILES: COc1cccc(F)c1C1CCN(c2ccnc(Cl)c2Cl)CC1]

Intermediate I-67 was synthesized following the same approach described for intermediate I-64. Starting from I-29 (0.35 g, 1.67 mmol) and I-1 (0.46 g, 1.67 mmol), intermediate I-67 was obtained (0.21 g, 35.5%).

### Intermediate I-68

### [3′-Chloro-4-(2-fluoro-6-methoxy-phenyl)-3,4,5,6-tetrahydro-2H-[1,4′]bipyridinyl-2′-yl]-hydrazine (I-68)

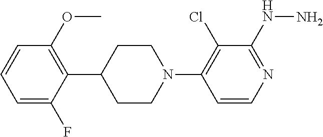 [SMILES: COc1cccc(F)c1C1CCN(c2ccnc(NN)c2Cl)CC1]

Intermediate I-68 was synthesized following the same approach described for intermediate I-65. Starting from I-67 (0.21 g, 0.59 mmol) and hydrazine hydrate (0.57, 11.88 mmol), intermediate I-68 was obtained (0.11 g, 52.3%).

### Intermediate I-69

### 3,3,3-Trifluoro-propionic acid N′-[3 ‘-chloro-4-(2-fluoro-6-methoxy-phenyl)-3,4,5,6-tetrahydro-2H-[1,4′]bipyridinyl-2’-yl]-hydrazide (I-69)

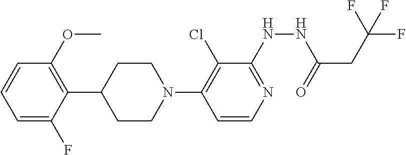 [SMILES: COc1cccc(F)c1C1CCN(c2ccnc(NNC(=O)CC(F)(F)F)c2Cl)CC1]

Intermediate I-69 was synthesized following the same approach reported for intermediate I-66. Starting from intermediate I-68 (0.11 g, 0.31 mmol) and 3,3,3-trifluoropropionyl chloride [C.A.S. 41463-83-6] (0.065 mL, 0.47 mmol), intermediate I-69 (0.144 g, quant. yield) was obtained.

### B. Preparation of the Final Compounds

### Example B1

### 8-Chloro-7-[4-(5-fluoro-2-methoxyphenyl)-1-piperidinyl]-3-(2,2,2-trifluoroethyl)-1,2,4-triazolo[4,3-a]pyridine (B-1)

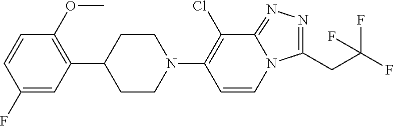 [SMILES: COc1ccc(F)cc1C1CCN(c2ccn3c(CC(F)(F)F)nnc3c2Cl)CC1]

To a solution of intermediate I-66 (0.35 g, 0.77 mmol) dissolved in CH₃CN (4 mL), POCl₃ [C.A.S. 10025-87-3] (0.09 mL, 1 mmol) was added. The r.m. was heated under microwave irradiation at 160° C. for 10 min. Then more POCl₃ (1 eq.) was added and the r.m. was heated again in a microwave oven at 150° C. for 5 min (cycle repeated twice). The mixture was then quenched with NaHCO₃ (sat. aq. sol.) and extracted with DCM. The organic layer was separated, dried (Na₂SO₄), filtered and the solvent evaporated in vacuo. The crude compound was purified by column chromatography (silica gel, EtOAc in DCM 0/100 to 15/85) the desired fractions were collected, the solvent evaporated in vacuo to yield compound B-1 as off-white solid (0.11 g, 33.5%). ¹H NMR (500 MHz, CDCl₃) δ ppm 1.89 (qd, J=12.4, 3.8 Hz, 2H), 1.94-2.00 (m, 2H), 3.08 (td, J=11.8, 2.3 Hz, 2H), 3.15 (tt, J=11.9, 3.4 Hz, 1H), 3.73-3.79 (m, 2H), 3.83 (s, 3H), 4.02 (q, J=9.8 Hz, 2H), 6.80 (dd, J=9.0, 4.6 Hz, 1H), 6.85 (d, J=7.5 Hz, 1H), 6.86-6.91 (m, 1H), 6.97 (dd, J=9.5, 3.2 Hz, 1H), 7.86 (d, J=7.2 Hz, 1H).

### Example B-2

### 8-Chloro-3-(cyclopropylmethyl)-7-[4-(3,6-difluoro-2-methoxyphenyl)-1-piperidinyl]-1,2,4-triazolo[4,3-a]pyridine (B-2)

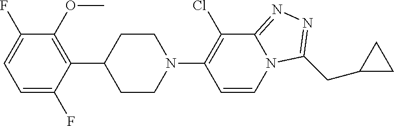 [SMILES: COc1c(F)ccc(F)c1C1CCN(c2ccn3c(CC4CC4)nnc3c2Cl)CC1]

To a mixture of intermediates I-4 (0.25 g, 0.75 mmol) and I-15 (0.22 g, 0.97 mmol) in toluene (2.5 mL), Pd(OAc)₂ (0.008 g, 0.04 mmol), (±)BINAP [C.A.S. 98327-87-8] (0.046 g, 0.07 mmol) and Cs₂CO₃ (0.37 g, 1.12 mmol) were added. The r.m. was heated at 125° C. overnight. Then DCM was added, the solid was filtered off, the filtrate solvent evaporated in vacuo, and the crude material purified by column chromatography (MeOH in DCM 0/100 to 5/95). The desired fractions were collected, the solvent evaporated in vacuo, and the solid material obtained was then washed with Et₂O to yield compound B-2 as off-white solid (0.19 g, 59.2%).

¹H NMR (500 MHz, CDCl₃) δ ppm 0.20-0.38 (m, 2H), 0.47-0.67 (m, 2H), 1.13-1.20 (m, 1H), 1.78 (br. d, J=12.4 Hz, 2H), 2.41 (qd, J=12.5, 2.7 Hz, 2H), 3.01 (t, J=12.1 Hz, 2H), 3.05 (d, J=6.9 Hz, 2H), 3.25 (tt, J=12.5, 3.4 Hz, 1H), 3.72 (br. d, J=11.8 Hz, 2H), 3.95 (d, J=1.7 Hz, 3H), 6.74 (td, J=9.2, 4.0 Hz, 1H), 6.76 (d, J=7.5 Hz, 1H), 6.93 (ddd, J=10.5, 9.2, 5.1 Hz, 1H), 7.84 (d, J=7.5 Hz, 1H).

### Example B-3

### 3-(Cyclopropylmethyl)-7-[4-(3,6-difluoro-2-methoxyphenyl)-1-piperidinyl]-8-(trifluoromethyl)-1,2,4-triazolo[4,3,-a]pyridine (B-3)

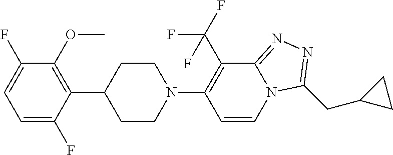 [SMILES: COc1c(F)ccc(F)c1C1CCN(c2ccn3c(CC4CC4)nnc3c2C(F)(F)F)CC1]

A mixture of intermediates I-10 (0.3 g, 1.09 mmol) and I-15 (0.37 g, 1.63 mmol) and DIPEA (0.38 mL, 2.18 mmol) was heated under microwave irradiation at 190° C. for 20 min. Then the solvent was evaporated and the crude material purified by column chromatography (EtOAc in DCM 0/100 to 100/0), the desired fractions were collected, the solvent evaporated in vacuo. The solid compound obtained was then washed with DIPE to yield compound B-3 as off-white solid (0.25 g, 48.2%). ¹H NMR (500 MHz, CDCl₃) δ ppm 0.28-0.38 (m, 2H), 0.57-0.67 (m, 2H), 1.11-1.20 (m, 1H), 1.75 (dd, J=12.1, 1.7 Hz, 2H), 2.35 (qd, J=12.4, 3.2 Hz, 2H), 3.04 (d, J=6.6 Hz, 2H), 3.18 (br. t, J=12.4 Hz, 2H), 3.27 (tt, J=12.4, 3.6 Hz, 1H), 3.62 (br. d, J=12.7 Hz, 2H), 3.94 (d, J=2.0 Hz, 3H), 6.72 (ddd, J=9.8, 9.3, 4.1 Hz, 1H), 6.75 (d, J=7.5 Hz, 1H), 6.93 (ddd, J=10.8, 9.2, 4.9 Hz, 1H), 7.91 (d, J=7.5 Hz, 1H).

### Example B-4

### 8-Chloro-3-(cyclopropylmethyl)-7-[4-(5-fluoro-2-methoxyphenyl)-1-piperidinyl]-1,2,4-triazolo[4,3-a]pyridine (B-4)

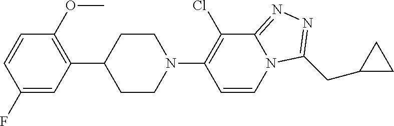 [SMILES: COc1ccc(F)cc1C1CCN(c2ccn3c(CC4CC4)nnc3c2Cl)CC1]

A suspension of intermediates I-4 (0.1 g, 0.3 mmol) and I-30 (0.13 g, 0.6 mmol) and NaHCO₃(0.061 g, 0.75 mmol) in CH₃CN (1 mL) was heated in a pressure tube (Q-Tube™) at 180° C. overnight. Then the r.m. was diluted with DCM and HCl (2N in H₂O), the organic layer separated, dried (Na₂SO₄), and the solvent evaporated in vacuo. The crude material was purified by column chromatography (EtOAc in DCM 0/100 to 100/0), the desired fractions were collected and the solvent evaporated in vacuo. The solid compound obtained was then washed with DIPE to yield compound B-4 as off-white solid (0.06 g, 49%). ¹H NMR (500 MHz, CDCl₃) δ ppm 0.27-0.38 (m, 2H), 0.55-0.67 (m, 2H), 1.13-1.20 (m, 1H), 1.89 (qd, J=12.1, 3.8 Hz, 2H), 1.93-1.99 (m, 2H), 3.00-3.07 (m, 2H), 3.05 (d, J=6.6 Hz, 2H), 3.14 (tt, J=11.7, 3.6 Hz, 1H), 3.71 (br. d, J=11.8 Hz, 2H), 3.83 (s, 3H), 6.76 (d, J=7.5 Hz, 1H), 6.80 (dd, J=9.0, 4.6 Hz, 1H), 6.86-6.92 (m, 1H), 6.97 (dd, J=9.5, 3.2 Hz, 1H), 7.84 (d, J=7.5 Hz, 1H).

### Example B-5

### 8-Chloro-7-[4-(2-fluoro-6-methoxyphenyl)-1-piperidinyl]-3-(2,2,2-trifluoroethyl)-1,2,4-triazolo[4,3-a]pyridine (B-5)

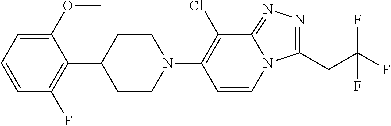 [SMILES: COc1cccc(F)c1C1CCN(c2ccn3c(CC(F)(F)F)nnc3c2Cl)CC1]

Compound B-5 was synthesized following the same methodology described for B-1. Starting from intermediate I-69 (0.1 g, 0.13 mmol) and treated with POCl₃ [C.A.S. 10025-87-3] (0.04 mL, 0.43 mmol), compound B-5 was obtained as off-white solid (0.058 g, 61%). ¹H NMR (500 MHz, CDCl₃) δ ppm 1.71-1.81 (m, 2H), 2.45 (qd, J=12.4, 3.3 Hz, 2H), 3.04 (br. t, J=11.8, 2H), 3.33 (tt, J=12.4, 3.5 Hz, 1H), 3.77 (br. d, J=11.8 Hz, 2H), 3.85 (s, 3H), 4.02 (q, J=9.8 Hz, 2H), 6.66-6.72 (m, 2H), 6.85 (d, J=7.5 Hz, 1H), 7.15 (td, J=8.3, 6.5 Hz, 1H), 7.85 (d, J=7.5 Hz, 1H).

### Example B-6

### 8-Chloro-3-(cyclopropylmethyl)-7-[4-(2-fluoro-6-methoxyphenyl)-1-piperidinyl]-1,2,4-triazolo[4,3-a]pyridine (B-6)

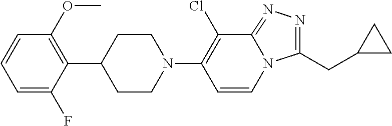 [SMILES: COc1cccc(F)c1C1CCN(c2ccn3c(CC4CC4)nnc3c2Cl)CC1]

Compound B-6 was synthesized following a similar approach to that described for B-4 changing the heating system from pressure tube to microwave irradiation (230° C., 30 min). Starting from intermediate I-4 (0.1 g, 0.3 mmol) and intermediate I-29 (0.094 g, 0.45 mmol), final product B-6 was obtained as off-white solid (0.05 g, 38.5%). ¹H NMR (500 MHz, CDCl₃) δ ppm 0.28-0.38 (m, 2H), 0.56-0.66 (m, 2H), 1.13-1.22 (m, 1H), 1.71-1.78 (m, 2H), 2.45 (qd, J=12.3, 3.2 Hz, 2H), 3.01 (br. t, J=11.8 Hz, 2H), 3.05 (d, J=6.6 Hz, 2H), 3.31 (tt, J=12.3, 3.5 Hz, 1H), 3.72 (br. d, J=11.8 Hz, 2H), 3.85 (s, 3H), 6.66-6.72 (m, 2H), 6.77 (d, J=7.5 Hz, 1H), 7.15 (td, J=8.3, 6.5 Hz, 1H), 7.83 (d, J=7.5 Hz, 1H).

### Example B-7

### 8-Chloro-3-(cyclopropylmethyl)-7-[4-(2,4-difluoro-6-methoxyphenyl)-1-piperidinyl]-1,2,4-triazolo[4,3-a]pyridine (B-7)

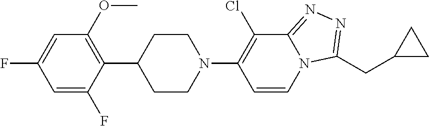 [SMILES: COc1cc(F)cc(F)c1C1CCN(c2ccn3c(CC4CC4)nnc3c2Cl)CC1]

Compound B-7 was synthesized following the same approach described for B-2. Starting from intermediate I-4 (0.1 g, 0.3 mmol) and intermediate I-31 (0.08 g, 0.36 mmol), compound B-7 was obtained as off-white solid (0.05 g, 38%). ¹H NMR (500 MHz, CDCl₃) δ ppm 0.28-0.38 (m, 2H), 0.55-0.67 (m, 2H), 1.12-1.21 (m, 1H), 1.68-1.76 (m, 2H), 2.40 (qd, J=12.3, 3.3 Hz, 2H), 2.98 (br. t, J=11.7 Hz, 2H), 3.05 (d, J=6.6 Hz, 2H), 3.22 (tt, J=12.4, 3.6 Hz, 1H), 3.70 (br. d, J=11.8 Hz, 2H), 3.84 (s, 3H), 6.39-6.47 (m, 2H), 6.76 (d, J=7.5 Hz, 1H), 7.83 (d, J=7.5 Hz, 1H).

### Example B-8

### 8-Chloro-3-(cyclopropylmethyl)-7-[4-(3,4-difluoro-2-methoxyphenyl)-1-piperidinyl]-1,2,4-triazolo[4,3-a]pyridine (B-8)

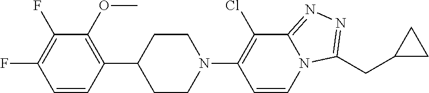 [SMILES: COc1c(C2CCN(c3ccn4c(CC5CC5)nnc4c3Cl)CC2)ccc(F)c1F]

Compound B-8 was synthesized following the same approach described for compound B-2. Starting from intermediates I-4 (0.15 g, 0.45 mmol) and I-33 (0.12 g, 0.54 mmol), compound B-8 was obtained as off-white solid (0.042 g, 21%). ¹H NMR (400 MHz, CDCl₃) δ ppm 0.26-0.39 (m, 2H), 0.54-0.68 (m, 2H), 1.11-1.23 (m, 1H), 1.85-2.00 (m, 4H), 2.97-3.13 (m, 5H), 3.70 (br. d, J=11.8 Hz, 2H), 4.00 (d, J=2.1 Hz, 3H), 6.75 (d, J=7.4 Hz, 1H), 6.83-6.92 (m, 1H), 6.93-6.99 (m, 1H), 7.84 (d, J=7.4 Hz, 1H).

### Example B-9

### 8-Chloro-3-(cyclopropylmethyl)-7-[4-(3-fluoro-2-methoxyphenyl)-1-piperidinyl]-1,2,4-triazolo[4,3-a]pyridine (B-9)

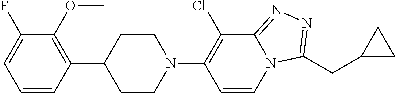 [SMILES: COc1c(F)cccc1C1CCN(c2ccn3c(CC4CC4)nnc3c2Cl)CC1]

Compound B-9 was synthesized following the same approach described for compound B-2. Starting from intermediates I-4 (0.1 g, 0.3 mmol) and I-57 (0.075 g, 0.36 mmol), final product B-9 was obtained as off-white solid (0.025 g, 19.5%). ¹H NMR (400 MHz, CDCl₃) δ ppm 0.26-0.39 (m, 2H), 0.54-0.68 (m, 2H), 1.11-1.23 (m, 1H), 1.86-2.04 (m, 4H), 2.98-3.10 (m, 4H), 3.11-3.21 (m, 1H), 3.67-3.75 (m, 2H), 3.95 (d, J=1.8 Hz, 3H), 6.77 (d, J=7.4 Hz, 1H), 6.94-7.09 (m, 3H), 7.85 (d, J=7.4 Hz, 1H).

### Example B-10

### 8-Chloro-3-(cyclopropylmethyl)-7-[4-(2,3-difluoro-6-methoxyphenyl)-1-piperidinyl]-1,2,4-triazolo[4,3-a]pyridine (B-10)

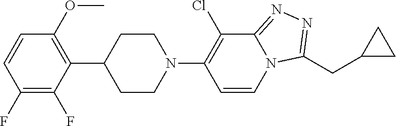 [SMILES: COc1ccc(F)c(F)c1C1CCN(c2ccn3c(CC4CC4)nnc3c2Cl)CC1]

Compound B-10 was synthesized following the same approach described for compound B-2. Starting from intermediates I-4 (0.1 g, 0.3 mmol) and I-32 (0.08 g, 0.36 mmol), compound B-10 was obtained as off-white solid (0.04 g, 27.6%). ¹H NMR (400 MHz, CDCl₃) δ ppm 0.26-0.39 (m, 2H), 0.54-0.68 (m, 2H), 1.11-1.22 (m, 1H), 1.71-1.80 (m, 2H), 2.45 (qd, J=12.4, 3.4 Hz, 2H), 3.00 (br. t, J=11.4, 2H), 3.05 (d, J=6.7 Hz, 2H), 3.30 (tt, J=12.4, 3.5 Hz, 1H), 3.67-3.75 (m, 2H), 3.83 (s, 3H), 6.52-6.61 (m, 1H), 6.76 (d, J=7.6 Hz, 1H), 6.98 (q, J=9.2 Hz, 1H), 7.83 (d, J=7.6 Hz, 1H).

### Example B-11

### 8-Chloro-3-(cyclopropylmethyl)-7-[4-(2-methoxyphenyl)-1-piperidinyl]-1,2,4-triazolo[4,3-a]pyridine (B-11)

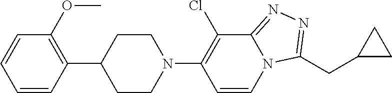 [SMILES: COc1ccccc1C1CCN(c2ccn3c(CC4CC4)nnc3c2Cl)CC1]

Compound B-11 was synthesized following the same approach described for compound B-2. Starting from intermediates I-4 (0.15 g, 0.45 mmol) and 4-(2-methoxyphenyl)piperidine [C.A.S. 58333-75-8] (0.1 g, 0.54 mmol), compound B-11 was obtained as off-white solid (0.056 g, 29.5%). ¹H NMR (400 MHz, CDCl₃) δ ppm 0.25-0.39 (m, 2H), 0.54-0.67 (m, 2H), 1.10-1.23 (m, 1H), 1.87-2.03 (m, 4H), 3.00-3.09 (m, 4H), 3.11-3.21 (m, 1H), 3.71 (br. d, J=12.5 Hz, 2H), 3.86 (s, 3H), 6.77 (d, J=7.4 Hz, 1H), 6.89 (br. d, J=8.1 Hz, 1H), 6.97 (br. t, J=7.4, 7.4 Hz, 1H), 7.19-7.24 (m, 1H), 7.25-7.29 (m, 1H), 7.84 (d, J=7.6 Hz, 1H).

### Example B-12

### 3-(Cyclopropylmethyl)-7-[4-(3-fluoro-2-methoxyphenyl)-1-piperidinyl]-8-(trifluoromethyl)-1,2,4-triazolo[4,3-a]pyridine (B-12)

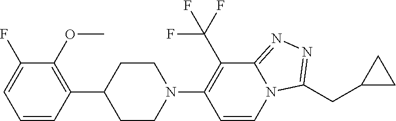 [SMILES: COc1c(F)cccc1C1CCN(c2ccn3c(CC4CC4)nnc3c2C(F)(F)F)CC1]

Compound B-12 was synthesized following the same approach described for compound B-3. Starting from intermediates I-10 (0.1 g, 0.36 mmol) and I-57 (0.09 g, 0.44 mmol), compound B-12 was obtained as off-white solid (0.045 g, 27.6%). ¹H NMR (500 MHz, CDCl₃) δ ppm 0.28-0.39 (m, 2H), 0.56-0.68 (m, 2H), 1.08-1.20 (m, 1H), 1.82-1.97 (m, 4H), 3.05 (d, J=6.6 Hz, 2H), 3.10-3.18 (m, 1H), 3.18-3.28 (m, 2H), 3.60 (br. d, J=13.0 Hz, 2H), 3.95 (d, J=1.7 Hz, 3H), 6.77 (d, J=7.8 Hz, 1H), 6.92-7.07 (m, 3H), 7.93 (d, J=7.8 Hz, 1H).

### Radiosynthesis

### Materials and Methods

HPLC analysis was performed on a LaChrom Elite HPLC pump (Hitachi, Darmstadt, Germany) connected to a UV spectrometer (Hitachi) set at 254 nm. For the analysis of radiolabeled compounds, the HPLC eluate after passage through the UV detector was led over a 7.62 cm (3 inch) NaI(Tl) scintillation detector connected to a single channel analyzer (Medi-Laboratory Select, Mechelen, Belgium). The radioactivity measurements during biodistribution studies and in vivo stability analyses were done using an automatic gamma counter (with a 3 in. NaI(Tl) well crystal) coupled to a multichannel analyzer (Wallac 1480 Wizard 3″, Wallac, Turku, Finland).

### Preparation of [¹¹C]B-2, [¹¹C]B-3, [¹¹C]B-4, [¹¹C]B-6, [¹¹C]B-7 and [¹¹C]B-10

Carbon-11 was produced using a Cyclone 18/9 cyclotron (Ion Beam Applications, Louvain-la-Neuve, Belgium) via a [¹⁴N(p,α)¹¹C] nuclear reaction. The target gas, which was a mixture of N₂ (95%) and H₂ (5%) was irradiated using 18 MeV protons at a beam current of 25 μA. The irradiation was done for about 30 min to yield [¹¹C] methane ([¹¹C]CH₄). The [¹¹C]CH₄ was then transferred to a home-built recirculation synthesis module and trapped on a Porapak® column that was immersed in liquid nitrogen. After flushing with helium, the condensed [¹¹C]CH₄ was converted to the gaseous phase by bringing the Porapak® loop to room temperature. This [¹¹C]CH₄ was then reacted with vaporous I₂ at 650° C. to convert it to [¹¹C]methyl iodide ([¹¹C]MeI). The resulting volatile [¹¹C]MeI was bubbled with a flow of helium through a solution of radiolabeling precursor I-58 (for [¹¹C]B-4), I-59 (for [¹¹C]B-6, I-62 (for [¹¹C]B-2), I-61 (for [¹¹C]B-7), I-60 (for [¹¹C]B-10), I-63 (for [¹¹C]B-3) (0.2 mg) and Cs₂CO₃ (1-3 mg) in anhydrous DMF (0.2 mL). When the amount of radioactivity in the reaction vial had stabilized, the reaction mixture was heated at 90° C. for 3 min. After dilution, the crude reaction mixture was injected onto an HPLC system consisting of a semi-preparative XBridge® column (C₁₈, 5 μm; 4.6 mm×150 mm; Waters, Milford, Mass., USA) that was eluted with a mixture of 0.05 M sodium acetate buffer (pH 5.5) and EtOH (50:50 v/v) at a flow rate of 1 mL/min. UV detection was done at 254 nm. The radiolabeled product was collected between 12 and 16 min (small difference in Rₜ time for the different tracers). The collected peak corresponding to the desired radioligand was then diluted with saline (Mini Plasco®, Braun, Melsungen, Germany) to obtain a final EtOH concentration of 10% and the solution was sterile filtered through 0.22 μm membrane filter (Millex®-GV, Millipore, Ireland). This formulation was then used for all in vivo experiments. The purity of the radiotracer was analyzed using an analytical HPLC system consisting of an XBridge column (C₁₈, 3.5 μm; 3 mm×100 mm; Waters) eluted with a mixture of 0.05 M NaOAc buffer (pH 5.5) and CH₃CN (55:45 v/v) at a flow rate of 0.8 mL/min (Rt=4-7 min, small difference in Rₜ for the different tracers).
[¹¹C]B-2 was synthesized in 74% radiochemical yield (n=13),
[¹¹C]B-3 was synthesized in 74% radiochemical yield (n=4),
[¹¹C]B-4 was synthesized in 44% radiochemical yield (n=7),
[¹¹C]B-6 was synthesized in 35% radiochemical yield (n=3),
[¹¹C]B-7 was synthesized in 61% radiochemical yield (n=5),
[¹¹C]B-10 was synthesized in 59% radiochemical yield (n=4).
All yields are determined relative to [¹¹C]MeI starting radioactivity, non-decay corrected. All radioligands were obtained with radiochemical purity >95% as examined using the above described analytical HPLC system.

The identity of the radiotracers was confirmed using the same analytical HPLC method as described above after co-injection with their non-radioactive analogue.

### C. Analytical Part

### Melting Points (mp):

Values are peak values, and are obtained with experimental uncertainties that are commonly associated with this analytical method.

For a number of compounds, noted as “DSC” in the table below, melting points were determined with a DSC823e (Mettler-Toledo). Melting points were measured with a temperature gradient of 30° C./minute. Maximum temperature was 400° C.

For a number of compounds, melting points were determined in open capillary tubes on a Mettler FP62 apparatus. Melting points were measured with a temperature gradient of 10° C./minute. Maximum temperature was 300° C. The melting point was read from a digital display.

Nuclear Magnetic Resonance (NMR)

¹H NMR spectra were recorded either on a Bruker DPX-400 or on a Bruker AV-500 spectrometer with standard pulse sequences, operating at 400 MHz and 500 MHz respectively. Chemical shifts (δ) are reported in parts per million (ppm) downfield from tetramethylsilane (TMS), which was used as internal standard.

### LCMS-Methods:

For LCMS-characterization of the compounds of the present invention, the following methods were used.

### General Procedure A

The HPLC measurement was performed using an HP 1100 (Agilent Technologies) system comprising a pump (quaternary or binary) with degasser, an autosampler, a column oven, a diode-array detector (DAD) and a column as specified in the respective methods below. Flow from the column was split to the MS spectrometer. The MS detector was configured with either an electrospray ionization source or an ESCI dual ionization source (electrospray combined with atmospheric pressure chemical ionization). Nitrogen was used as the nebulizer gas. The source temperature was maintained at 140° C. Data acquisition was performed with MassLynx-Openlynx software.

### General Procedure B

The UPLC (Ultra Performance Liquid Chromatography) measurement was performed using an Acquity UPLC (Waters) system comprising a sampler organizer, a binary pump with degasser, a four column's oven, a diode-array detector (DAD) and a column as specified in the respective methods below. Column flow was used without split to the MS detector. The MS detector was configured with an ESCI dual ionization source (electrospray combined with atmospheric pressure chemical ionization). Nitrogen was used as the nebulizer gas. The source temperature was maintained at 140° C. Data acquisition was performed with MassLynx-Openlynx software.

### Method 1

In addition to the general procedure B: Reversed phase UPLC was carried out on a BEH-C18 column (1.7 μm, 2.1×50 mm) from Waters, with a flow rate of 0.8 ml/min, at 60° C. without split to the MS detector. The gradient conditions used are: 95% A (0.5 g/l ammonium acetate solution +5% acetonitrile), 5% B (mixture of acetonitrile/methanol, 1/1), to 20% A, 80% B in 4.9 minutes, to 100% B in 5.3 minutes, kept till 5.8 minutes and equilibrated to initial conditions at 6.0 minutes until 7.0 minutes. Injection volume 0.5 μl. Low-resolution mass spectra (single quadrupole, SQD detector) were acquired by scanning from 100 to 1000 in 0.1 seconds using an inter-channel delay of 0.08 second. The capillary needle voltage was 3 kV. The cone voltage was 20 V for positive ionization mode and 30 V for negative ionization mode.

### Method 2

In addition to the general procedure B: Reversed phase UPLC was carried out on a BEH-C18 column (1.7 μm, 2.1×50 mm) from Waters, with a flow rate of 0.8 ml/min, at 60° C. without split to the MS detector. The gradient conditions used are: 95% A (0.5 g/l ammonium acetate solution +5% acetonitrile), 5% B (mixture of acetonitrile/methanol, 1/1), kept 0.2 minutes, to 20% A, 80% B in 3.5 minutes, to 100% B in 3.8 minutes, kept till 4.15 minutes and equilibrated to initial conditions at 4.3 minutes until 5.0 minutes. Injection volume 0.5 μl. Low-resolution mass spectra (single quadrupole, SQD detector) were acquired by scanning from 100 to 1000 in 0.1 seconds using an inter-channel delay of 0.08 second. The capillary needle voltage was 3 kV. The cone voltage was 20 V for positive ionization mode and 30 V for negative ionization mode.

### Method 3

In addition to the general procedure B: Reversed phase UPLC was carried out on a BEH-C18 column (1.7 μm, 2.1×50 mm) from Waters, with a flow rate of 1.0 ml/min, at 50° C. without split to the MS detector. The gradient conditions used are: 95% A (0.5 g/l ammonium acetate solution +5% acetonitrile), 5% B (acetonitrile), to 40% A, 60% B in 4.4 minutes, to 5% A, 95% B in 5.6 minutes, kept till 5.8 minutes and equilibrated to initial conditions at 6.0 minutes until 7.0 minutes. Injection volume 0.5 μl. Low-resolution mass spectra (single quadrupole, SQD detector) were acquired by scanning from 100 to 1000 in 0.1 seconds using an inter-channel delay of 0.08 second. The capillary needle voltage was 3 kV. The cone voltage was 25 V for positive ionization mode and 30 V for negative ionization mode.

### Method 4

In addition to the general procedure B: Reversed phase UPLC was carried out on a BEH-C18 column (1.7 μm, 2.1×50 mm) from Waters, with a flow rate of 1.0 ml/min, at 50° C. without split to the MS detector. The gradient conditions used are: 95% A (0.5 g/l ammonium acetate solution +5% acetonitrile), 5% B (acetonitrile), to 40% A, 60% B in 2.8 minutes, to 5% A, 95% B in 3.6 minutes, kept till 3.8 minutes and equilibrated to initial conditions at 4.0 minutes until 5.0 minutes. Injection volume 0.5 μl. Low-resolution mass spectra (single quadrupole, SQD detector) were acquired by scanning from 100 to 1000 in 0.1 seconds using an inter-channel delay of 0.08 second. The capillary needle voltage was 3 kV. The cone voltage was 25 V for positive ionization mode and 30 V for negative ionization mode.

### Method 5

In addition to the general procedure A: Reversed phase HPLC was carried out on an Eclipse Plus-C18 column (3.5 μm, 2.1×30 mm) from Agilent, with a flow rate of 1.0 ml/min, at 60° C. without split to the MS detector. The gradient conditions used are: 95% A (0.5 g/l ammonium acetate solution +5% acetonitrile), 5% B (mixture of acetonitrile/methanol, 1/1), to 100% B in 5.0 minutes, kept till 5.15 minutes and equilibrated to initial conditions at 5.30 minutes until 7.0 minutes. Injection volume 2 μl. Low-resolution mass spectra (single quadrupole, SQD detector) were acquired by scanning from 100 to 1000 in 0.1 second using an inter-channel delay of 0.08 second. The capillary needle voltage was 3 kV. The cone voltage was 20 V for positive ionization mode and 30 V for negative ionization mode.

### Method 6

In addition to the general procedure A: Reversed phase HPLC was carried out on a Sunfire-C18 column (2.5 μm, 2.1×30 mm) from Waters, with a flow rate of 1.0 ml/min, at 60° C. The gradient conditions used are: 95% A (0.5 g/l ammonium acetate solution +5% of acetonitrile), 2.5% B (acetonitrile), 2.5% C (methanol) to 50% B, 50% C in 6.5 minutes, kept till 7.0 minutes and equilibrated to initial conditions at 7.3 minutes until 9.0 minutes. Injection volume 2 μl. High-resolution mass spectra (Time of Flight, TOF detector) were acquired by scanning from 100 to 750 in 0.5 seconds using a dwell time of 0.3 seconds. The capillary needle voltage was 2.5 kV for positive ionization mode and 2.9 kV for negative ionization mode. The cone voltage was 20 V for both positive and negative ionization modes. Leucine-Enkephaline was the standard substance used for the lock mass calibration.

TABLE I
Compounds of formula (I)

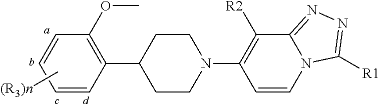

| Co. | | | | mp | | | LCMS |
|---|---|---|---|---|---|---|---|
| No. | R¹ | R² | (R³)ₙ | (° C.) | [MH⁺] | Rₜ | (method) |
| 1 | —CH₂—CF₃ | —Cl | c-F | 259 | 443 | 3.87 | 5 |
| 2 | 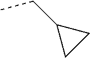 | —Cl | a-F d-F | 163.2 | 433 | 2.43 | 4 |
| 3 | 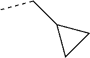 | —CF₃ | a-F d-F | 180.7 | 467 | 3.56 | 2 |
| 4 | 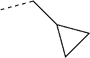 | —Cl | c-F | >300 | 415 | 3.43 | 3 |
| 5 | —CH₂—CF₃ | —Cl | d-F | Foam | 443 | 3.47 | 3 |
| 6 | 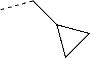 | —Cl | d-F | Foam | 415 | 4.72 | 6 |
| 7 | 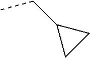 | —Cl | b-F d-F | 162.2 | 433 | 3.6 | 3 |
| 8 | 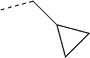 | —Cl | a-F b-F | 173.5 | 433 | 3.56 | 3 |
| 9 | 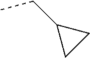 | —Cl | a-F | 168.3 | 415 | 3.40 | 3 |
| 10 | 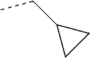 | —Cl | c-F d-F | 208.5 | 433 | 3.48 | 3 |
| 11 | 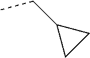 | —Cl | — | Foam | 397 | 3.38 | 3 |
| 12 | 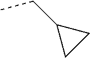 | —CF₃ | a-F | 195.7 | 449 | 3.57 | 3 |

Analytical data (Rₜ means retention time in minutes; [MH⁺] means the protonated mass of the compound; LCMS procedure refers to the method used for LCMS).

### II. [³⁵S]GTPγS Binding Assay

The compounds provided in the present invention are positive allosteric modulators of mGluR2. These compounds appear to potentiate glutamate responses by binding to an allosteric site other than the glutamate binding site. The response of mGluR2 to a concentration of glutamate is increased when compounds of Formula (I) are present. Compounds of Formula (I) are expected to have their effect substantially at mGluR2 by virtue of their ability to enhance the function of the receptor. The effects of positive allosteric modulators tested at mGluR2 using the [³⁵S]GTPγS binding assay method described below and which is suitable for the identification of such compounds, and more particularly the compounds according to Formula (I), is shown in Table II.

### [³⁵S]GTPγS Binding Assay

The [³⁵S]GTPγS binding assay is a functional membrane-based assay used to study G-protein coupled receptor (GPCR) function whereby incorporation of a non-hydrolysable form of GTP, [³⁵S]GTPγS (guanosine 5′-triphosphate, labelled with gamma-emitting ³⁵S), is measured. The G-protein α subunit catalyzes the exchange of guanosine 5′-diphosphate (GDP) by guanosine triphosphate (GTP) and on activation of the GPCR by an agonist, [³⁵S]GTPγS, becomes incorporated and cannot be cleaved to continue the exchange cycle (Harper (1998) Current Protocols in Pharmacology 2.6.1-10, John Wiley & Sons, Inc.). The amount of radioactive [³⁵S]GTPγS incorporation is a direct measure of the activity of the G-protein and hence the activity of the agonist can be determined mGluR2 receptors are shown to be preferentially coupled to Gαi-protein, a preferential coupling for this method, and hence it is widely used to study receptor activation of mGluR2 receptors both in recombinant cell lines and in tissues. Here we describe the use of the [³⁵S]GTPγS binding assay using membranes from cells transfected with the human mGluR2 receptor and adapted from Schaffhauser et al. ((2003) Molecular Pharmacology 4:798-810) for the detection of the positive allosteric modulation (PAM) properties of the compounds of this invention.

### Membrane Preparation

CHO-cells were cultured to pre-confluence and stimulated with 5 mM butyrate for 24 h. Cells were then collected by scraping in PBS and cell suspension was centrifuged (10 min at 4000 RPM in benchtop centrifuge). Supernatant was discarded and pellet gently resuspended in 50 mM Tris-HCl, pH 7.4 by mixing with a vortex and pipetting up and down. The suspension was centrifuged at 16,000 RPM (Sorvall RC-5C plus rotor SS-34) for 10 minutes and the supernatant discarded. The pellet was homogenized in 5 mM Tris-HCl, pH 7.4 using an ultra-turrax homogenizer and centrifuged again (18,000 RPM, 20 min, 4° C.). The final pellet was resuspended in 50 mM Tris-HCl, pH 7.4 and stored at −80° C. in appropriate aliquots before use. Protein concentration was determined by the Bradford method (Bio-Rad, USA) with bovine serum albumin as standard.

### [³⁵S]GTPγS Binding Assay

Measurement of mGluR2 positive allosteric modulatory activity of test compounds was performed as follows. Test compounds and glutamate were diluted in assay buffer containing 10 mM HEPES acid, 10 mM HEPES salt, pH 7.4, 100 mM NaCl, 3 mM MgCl₂ and 10 μM GDP. Human mGlu2 receptor-containing membranes were thawed on ice and diluted in assay buffer supplemented with 14 μg/ml saponin. Membranes were pre-incubated with compound alone or together with a predefined (˜EC₂₀) concentration of glutamate (PAM assay) for 30 min at 30° C. After addition of [³⁵S]GTPγS (f.c. 0.1 nM), assay mixtures were shaken briefly and further incubated to allow [³⁵S]GTPγS incorporation on activation (30 minutes, 30° C.). Final assay mixtures contained 7 μg of membrane protein in 10 mM HEPES acid, 10 mM HEPES salt, pH 7.4, 100 mM NaCl, 3 mM MgCl₂,10 μM GDP and 10 μg/ml saponin. Total reaction volume was 200 μl. Reactions were terminated by rapid filtration through Unifilter-96 GF/B plates (Perkin Elmer, Mass., USA) using a 96-well filtermate universal harvester. Filters were washed 6 times with ice-cold 10 mM NaH₂PO₄/10 mM Na₂HPO₄, pH 7.4. Filters were then air-dried, and 40 μl of liquid scintillation cocktail (Microscint-O) was added to each well. Membrane-bound radioactivity was counted in a Microplate Scintillation and Luminescence Counter from Perkin Elmer.

### Data Analysis

The concentration-response curves of representative compounds of the present invention—obtained in the presence of EC₂₀ of mGluR2 agonist glutamate to determine positive allosteric modulation (PAM)—were generated using the Lexis software interface (developed at J&J). Data were calculated as % of the control glutamate response, defined as the maximal response that is generated upon addition of glutamate alone. Sigmoid concentration-response curves plotting these percentages versus the log concentration of the test compound were analyzed using non-linear regression analysis. The concentration producing half-maximal effect is then calculated as EC₅₀.

The pEC₅₀ values below were calculated as the −log EC₅₀, when the EC₅₀ is expressed in M.

Selectivity of the compounds for hmGluR2 versus hmGluR1, hmGluR3, hmGluR4, hmGluR5, rmGluR6, hmGluR7 and hmGluR8 was determined using functional receptor assays (either measuring changes in intracellular Ca2+ mobilization or G protein activation via [³⁵S]GTPγS) with cells overexpressing the receptor of interest. Table II below shows the pharmacological data obtained for compounds B1-B12.

TABLE II
Data in the [³⁵S]GTPγS binding assay and selectivity for mGluR2 versus
mGluR1, mGluR3-mGluR8.

| | GTPγS- | |
|---|---|---|
| | hmGluR2 | |
| Co. | PAM | |
| No. | pEC₅₀ | Selectivity over mGluR1, mGluR3-mGluR8 |
| 1 | 7.98 | >1,000 fold |
| 2 | 8.13 | >1,000 fold |
| 3 | 8.39 | >1,000 fold |
| 4 | 8.03 | >500, except for mGluR3 ≧40 fold |
| 5 | 8.41 | ≧1,000 fold |
| 6 | 8.22 | >1,000 fold, except for mGluR3 >300 fold, and |
| | | mGluR7 and mGluR8 for which selectivity >500 fold |
| 7 | 8.16 | >1,000 fold, except for mGluR3 and mGluR8 |
| | | ≧500 fold |
| 8 | 7.58 | >1,000 fold, except for mGluR3 ≧400 fold |
| 9 | 7.43 | >500 fold |
| 10 | 8.03 | >1,000 fold, except for mGluR3 and mGluR8 for |
| | | which selectivity ≧500 fold |
| 11 | 7.7 | >100, except for mGluR3 for which selectivity |
| | | ~20 fold |
| 12 | 7.95 | ≧1,000 fold, except for mGluR3 ≧200 fold |

pEC₅₀ values were calculated from a concentration-response experiment of at least 8 concentrations. If more experiments were performed, the average pEC₅₀ value is reported and error deviation was <0.5.

### III. Biodistribution Studies

### General Method

Biodistribution studies were carried out in healthy male Wistar rats (body weight 200-450 g) at 2 min, 30 min and 60 min post injection (p.i.) (n=3/time point). Rats were injected with about 11 MBq (2 min, 30 min analysis) or 22 MBq (60 min analysis) of the tracer via tail vein under anesthesia (2.5% isoflurane in O₂ at 1 L/min flow rate) and sacrificed by decapitation at above specified time points. Blood and major organs were collected in tared tubes and weighed. The radioactivity in blood, organs and other body parts was measured using an automated gamma counter. The distribution of radioactivity in different parts of the body at different time points p.i. of the tracer was calculated and expressed as percentage of injected dose (% ID), and as percentage of injected dose per gram tissue (% ID/g) for the selected organs. % ID is calculated as cpm in organ/total cpm recovered. For calculation of total radioactivity in blood, blood mass was assumed to be 7% of the body mass.

All animal experiments were conducted with the approval of the institutional ethical committee for conduct of experiments on animals.

III.a. Biodistribution Results for Compound [¹¹C]B-2

The results of the biodistribution study of [¹¹C]B-2 in male Wistar rats is presented in Tables 1 and 2. Table 1 shows the % ID values at 2 min, 30 min and 60 min p.i. of the radiotracer. The total initial brain uptake of the tracer was 0.88% of the ID, with 0.69% ID in the cerebrum and 0.17% ID in the cerebellum. At 2 min p.i. 4.3% of the injected dose was present in the blood, and this cleared to 2.0% by 60 min p.i. The tracer was cleared mainly by the hepatobiliary system as there was in total 35.7% of ID present in liver and intestines 60 min after injection of the radiotracer. Because of its lipophilic character, the urinary excretion of the tracer was minimal with only 2.4% ID present in the urinary system at 60 min p.i. In view of the large mass of the carcass, significant amount of the injected dose (˜50% ID) was present in the carcass at all time points examined. Typically, carcass constitutes ≧90% of the total body weight of the animal.

TABLE 1
Biodistribution of [¹¹C]B-2 in normal rats at 2, 30 and 60 min p.i.

| | % ID^{a} | | |
|---|---|---|---|
| Organ | 2 min | 30 min | 60 min |
| Urine | 0.1 ± 0.0 | 0.3 ± 0.1 | 0.3 ± 0.1 |
| Kidneys | 6.6 ± 0.7 | 4.3 ± 1.0 | 2.1 ± 0.2 |
| Liver | 33.5 ± 1.4 | 22.7 ± 3.0 | 20.1 ± 7.0 |
| Spleen + Pancreas | 1.4 ± 0.1 | 1.4 ± 0.2 | 0.7 ± 0.0 |
| Lungs | 1.5 ± 0.1 | 1.1 ± 0.5 | 0.6 ± 0.1 |
| Heart | 4.6 ± 0.6 | 2.5 ± 0.8 | 1.2 ± 0.2 |
| Stomach | 1.4 ± 0.2 | 3.7 ± 0.3 | 1.7 ± 0.4 |
| Intestines | 8.5 ± 0.3 | 10.4 ± 1.2 | 15.6 ± 2.7 |
| Striatum | 0.032 ± 0.008 | 0.047 ± 0.008 | 0.033 ± 0.008 |
| Hippocampus | 0.028 ± 0.008 | 0.045 ± 0.005 | 0.024 ± 0.006 |
| Cortex | 0.097 ± 0.019 | 0.118 ± 0.041 | 0.080 ± 0.022 |
| Rest of cerebrum | 0.535 ± 0.121 | 0.704 ± 0.112 | 0.421 ± 0.010 |
| Cerebrum total | 0.691 ± 0.146 | 0.914 ± 0.140 | 0.558 ± 0.042 |
| Cerebellum | 0.174 ± 0.039 | 0.291 ± 0.088 | 0.142 ± 0.029 |
| Blood | 4.3 ± 0.6 | 2.7 ± 0.9 | 2.0 ± 0.0 |
| Carcass | 38.4 ± 2.6 | 50.9 ± 3.4 | 55.8 ± 9.4 |

Data are expressed as mean ± SD;
n = 3 per time point;
^{a}Percentage of ID calculated as cpm in organ/total cpm recovered

In order to correct for differences in body weight between different animals, the % ID/g tissue values were normalized for body weight. The normalized values (SUV, standard uptake value) for striatum, hippocampus, cortex and cerebellum are presented in Table 2.

At 30 min p.i. the radioactivity concentration has increased for all brain regions. This accumulation of radioactivity in all studied brain regions is consistent with the fact that mGluR2 receptors are expressed in several brain areas including hippocampus, cortical regions, olfactory bulb, cerebellum and striatum. Most significant increase was observed for striatum (SUV 1.22 at 2 min p.i. to SUV 2.14 at 30 min p.i.), followed by cerebellum. The highest radioactivity concentration at 30 min is found in the cerebellum (SUV 2.62), followed by striatum. For all brain regions the radioactivity concentration at 60 min p.i. is lower compared to 30 min time point, indicating that wash-out has started.

TABLE 2
[¹¹C]B-2 concentration in different brain regions and blood at
2, 30 and 60 min p.i. normalized for the body weight of the animal

| | SUV^{a} | | |
|---|---|---|---|
| Organ | 2 min | 30 min | 60 min |
| Striatum | 1.22 ± 0.02 | 2.14 ± 0.04 | 1.72 ± 0.02 |
| Hippocampus | 0.90 ± 0.01 | 1.49 ± 0.03 | 0.73 ± 0.06 |
| Cortex | 1.46 ± 0.03 | 1.77 ± 0.04 | 1.28 ± 0.02 |
| Cerebrum total | 1.32 ± 0.03 | 1.96 ± 0.03 | 1.11 ± 0.06 |
| Cerebellum | 1.59 ± 0.03 | 2.62 ± 0.04 | 1.75 ± 0.04 |
| Blood | 0.60 ± 0.01 | 0.40 ± 0.01 | 0.30 ± 0.01 |

Data are expressed as mean ± SD;
n = 3 per time point;
^{a}SUV are calculated as (radioactivity in cpm in organ/weight of the organ in g)/(total counts recovered/body weight in g)

### III.b. [¹¹C]B-4

The results of the in vivo distribution study of [¹¹C]B-4 in male Wistar rats is presented in Tables 3 and 4. Table 3 shows the % ID values at 2 min, 30 min and 60 min p.i. of the radiotracer. At 2 min p.i. 5.6% of the ID was present in the blood, and this cleared to 3.3% by 60 min after injection of the tracer. The total initial brain uptake of the tracer was 0.58%, with 0.45% of the ID in the cerebrum and 0.10% in the cerebellum. At 60 min after injection of the radiotracer, 26.5% ID was present in the liver and intestines. Because of its lipophilic character, the urinary excretion of the tracer was minimal with only 3.5% ID present in the urinary system at 60 min p.i. In view of the large mass of the carcass, significant amount of the ID (˜56% ID) was present in the carcass at all time points examined. Typically, carcass constitutes ≧90% of the total body weight of the animal.

TABLE 3
Biodistribution of [¹¹C]B-4 in normal rats at 2, 30 and 60 min p.i.

| | % ID^{a} | | |
|---|---|---|---|
| Organ | 2 min | 30 min | 60 min |
| Urine | 0.0 ± 0.0 | 0.3 ± 0.2 | 0.6 ± 0.1 |
| Kidneys | 6.4 ± 0.6 | 4.0 ± 0.7 | 2.9 ± 0.4 |
| Liver | 29.9 ± 2.0 | 14.2 ± 2.4 | 15.4 ± 0.9 |
| Spleen + Pancreas | 1.9 ± 0.2 | 1.4 ± 0.2 | 1.3 ± 0.2 |
| Lungs | 2.9 ± 0.4 | 0.7 ± 0.1 | 0.5 ± 0.1 |
| Heart | 4.1 ± 0.3 | 2.5 ± 0.4 | 1.3 ± 0.3 |
| Stomach | 1.5 ± 0.3 | 2.1 ± 0.3 | 1.6 ± 0.3 |
| Intestines | 7.3 ± 0.6 | 8.3 ± 2.0 | 11.1 ± 0.9 |
| Striatum | 0.014 ± 0.001 | 0.034 ± 0.005 | 0.029 ± 0.009 |
| Hippocampus | 0.010 ± 0.001 | 0.026 ± 0.007 | 0.021 ± 0.005 |
| Cortex | 0.092 ± 0.019 | 0.165 ± 0.081 | 0.097 ± 0.039 |
| Rest of cerebrum | 0.344 ± 0.047 | 0.469 ± 0.047 | 0.450 ± 0.086 |
| Cerebrum total | 0.450 ± 0.029 | 0.694 ± 0.117 | 0.596 ± 0.128 |
| Cerebellum | 0.100 ± 0.000 | 0.191 ± 0.030 | 0.144 ± 0.021 |
| Blood | 5.6 ± 0.4 | 2.8 ± 0.3 | 3.3 ± 0.2 |
| Carcass | 42.0 ± 2.0 | 64.3 ± 5.8 | 62.8 ± 2.5 |

Data are expressed as mean ± SD;
n = 3 per time point;
^{a}Percentage of ID calculated as cpm in organ/total cpm recovered

In order to correct for differences in body weight between different animals, the % ID/g tissue values were normalized for body weight. The normalized values for striatum, hippocampus, cortex and cerebellum are presented in Table 4.

At 30 min p.i. the radioactivity concentration has increased for all brain regions. This accumulation of radioactivity in all studied brain regions is consistent with the fact that mGluR2 receptors are expressed in several brain areas including hippocampus, cortical regions, olfactory bulb, cerebellum and striatum. Most significant increase was observed for striatum and cerebellum (SUV 1.46 at 2 min p.i. to SUV 2.31 at 30 min p.i.). The highest radioactivity concentration at 30 min is found in the cerebellum and the striatum SUV ˜2.32), followed by the cortex. For all brain regions the radioactivity concentration at 60 min p.i. is lower compared to 30 min time point, indicating that wash-out has started.

TABLE 4
[¹¹C]B-4 concentration in different brain regions and blood at
2, 30 and 60 min p.i. normalized for the body weight of the animal

| | SUV^{a} | | |
|---|---|---|---|
| Organ | 2 min | 30 min | 60 min |
| Striatum | 1.46 ± 0.02 | 2.31 ± 0.04 | 1.78 ± 0.04 |
| Hippocampus | 1.04 ± 0.01 | 1.57 ± 0.03 | 1.13 ± 0.02 |
| Cortex | 1.65 ± 0.02 | 1.87 ± 0.02 | 1.34 ± 0.02 |
| Cerebrum total | 1.39 ± 0.01 | 1.66 ± 0.03 | 1.40 ± 0.03 |
| Cerebellum | 1.46 ± 0.02 | 2.33 ± 0.04 | 1.68 ± 0.04 |
| Blood | 0.80 ± 0.01 | 0.40 ± 0.01 | 0.50 ± 0.00 |

Data are expressed as mean ± SD;
n = 3 per time point;
^{a}SUV are calculated as (radioactivity in cpm in organ/weight of the organ in g)/(total counts recovered/body weight in g)

### III.c. [¹¹C]B-7

The results of the in vivo distribution study of [¹¹C]B-7 in male Wistar rats is presented in Tables 5 and 6. Table 5 shows the % ID values at 2 min, 30 min and 60 min p.i. of the radiotracer. At 2 min p.i. 5.4% of the ID was present in blood, and this cleared to 3.7% by 60 min after injection of the tracer. The total initial brain uptake of the tracer was 0.75%, with 0.53% of the ID in the cerebrum and 0.18% in the cerebellum. At 60 min after injection of the radiotracer, 28.7% ID was present in the liver and intestines. Because of its lipophilic character, the urinary excretion of the tracer was minimal with only 2.5% ID present in the urinary system at 60 min p.i. In view of the large mass of the carcass, significant amount of the ID (40% ID at 2 min, ˜62% ID at 30 and 60 min p.i.) was present in the carcass at all time points examined. Typically, carcass constitutes ≧90% of the total body weight of the animal.

TABLE 5
Biodistribution of [¹¹C]B-7 in normal rats at 2, 30 and 60 min p.i.

| | % ID^{a} | | |
|---|---|---|---|
| Organ | 2 min | 30 min | 60 min |
| Urine | 0.1 ± 0.0 | 0.4 ± 0.1 | 0.5 ± 0.2 |
| Kidneys | 6.5 ± 0.7 | 2.8 ± 0.3 | 2.0 ± 0.3 |
| Liver | 33.4 ± 2.0 | 14.6 ± 1.2 | 15.3 ± 2.1 |
| Spleen + Pancreas | 1.3 ± 0.2 | 1.2 ± 0.3 | 0.9 ± 0.0 |
| Lungs | 1.8 ± 0.7 | 0.6 ± 0.1 | 0.7 ± 0.1 |
| Heart | 4.1 ± 0.4 | 1.4 ± 0.2 | 0.9 ± 0.1 |
| Stomach | 1.5 ± 0.2 | 1.4 ± 0.2 | 2.4 ± 0.7 |
| Intestines | 8.2 ± 1.1 | 10.2 ± 1.5 | 13.4 ± 3.3 |
| Striatum | 0.028 ± 0.008 | 0.045 ± 0.014 | 0.026 ± 0.007 |
| Hippocampus | 0.020 ± 0.004 | 0.030 ± 0.003 | 0.022 ± 0.003 |
| Cortex | 0.081 ± 0.011 | 0.120 ± 0.018 | 0.059 ± 0.007 |
| Rest of cerebrum | 0.428 ± 0.084 | 0.523 ± 0.117 | 0.435 ± 0.004 |
| Cerebrum total | 0.529 ± 0.098 | 0.718 ± 0.142 | 0.543 ± 0.014 |
| Cerebellum | 0.179 ± 0.043 | 0.198 ± 0.026 | 0.163 ± 0.011 |
| Blood | 5.4 ± 0.3 | 3.5 ± 0.2 | 3.7 ± 0.4 |
| Carcass | 39.8 ± 2.8 | 64.9 ± 4.2 | 61.5 ± 5.7 |

Data are expressed as mean ± SD;
n = 3 per time point;
^{a}Percentage of ID calculated as cpm in organ/total cpm recovered

In order to correct for differences in body weight between different animals, the % ID/g tissue values were normalized for body weight. The normalized values for striatum, hippocampus, cortex and cerebellum are presented in Table 6.

At 30 min p.i. the radioactivity concentration has increased for all brain regions. This accumulation of radioactivity in all studied brain regions is consistent with the fact that mGluR2 receptors are expressed in several brain areas including hippocampus, cortical regions, olfactory bulb, cerebellum and striatum. Most significant increase was observed for striatum and cortex (SUV ˜1.13 at 2 min p.i. to SUV ˜1.71 at 30 min p.i.) The highest radioactivity concentration at 30 min is found in the cerebellum (SUV 2.0), followed by the cortex. For all brain regions the radioactivity concentration at 60 min p.i. is lower compared to 30 min time point, indicating that wash-out has started.

TABLE 6
[¹¹C]B-7 concentration in different brain regions and blood at
2, 30 and 60 min p.i. normalized for the body weight of the animal

| | SUV^{a} | | |
|---|---|---|---|
| Organ | 2 min | 30 min | 60 min |
| Striatum | 1.13 ± 0.03 | 1.70 ± 0.03 | 1.43 ± 0.01 |
| Hippocampus | 0.85 ± 0.02 | 1.20 ± 0.01 | 0.98 ± 0.01 |
| Cortex | 1.14 ± 0.03 | 1.72 ± 0.05 | 1.10 ± 0.01 |
| Cerebrum total | 1.08 ± 0.02 | 1.51 ± 0.03 | 1.19 ± 0.01 |
| Cerebellum | 1.53 ± 0.03 | 2.00 ± 0.03 | 1.50 ± 0.01 |
| Blood | 0.80 ± 0.00 | 0.50 ± 0.00 | 0.50 ± 0.01 |

Data are expressed as mean ± SD;
n = 3 per time point;
^{a}SUV are calculated as (radioactivity in cpm in organ/weight of the organ in g)/(total counts recovered/body weight in g)

### III.d. [¹¹C]B-6

The results of the in vivo distribution study of [¹¹C]B-6 in male Wistar rats is presented in Tables 7 and 8. Table 7 shows the % ID values at 2 min, 30 min and 60 min p.i. of the radiotracer. At 2 min p.i. 6.5% of the injected dose was present in the blood, and this cleared to 3.6% by 60 min after injection of the tracer. The total initial brain uptake of the tracer was 0.65%, with 0.45% of the ID in the cerebrum and 0.17% in the cerebellum. At 60 min after injection of the radiotracer, 30.6% ID was present in the liver and intestines. Because of its lipophilic character, the urinary excretion of the tracer was minimal with only 2.5% ID present in the urinary system at 60 min p.i. In view of the large mass of the carcass, significant amount of the ID (˜54%) was present in the carcass at all time points examined. Typically, carcass constitutes ≧90% of the total body weight of the animal.

TABLE 7
Biodistribution of [¹¹C]B-6 in normal rats at 2, 30 and 60 min p.i.

| | % ID^{a} | | |
|---|---|---|---|
| Organ | 2 min | 30 min | 60 min |
| Urine | 0.1 ± 0.0 | 0.3 ± 0.1 | 0.6 ± 0.1 |
| Kidneys | 6.8 ± 0.7 | 3.0 ± 0.4 | 1.9 ± 0.3 |
| Liver | 30.2 ± 0.9 | 17.0 ± 1.1 | 18.6 ± 1.0 |
| Spleen + Pancreas | 1.4 ± 0.1 | 1.0 ± 0.2 | 0.8 ± 0.0 |
| Lungs | 1.8 ± 0.5 | 0.8 ± 0.1 | 0.6 ± 0.1 |
| Heart | 4.1 ± 0.1 | 1.7 ± 0.2 | 1.0 ± 0.1 |
| Stomach | 1.3 ± 0.2 | 2.3 ± 0.5 | 4.3 ± 1.8 |
| Intestines | 7.6 ± 0.5 | 9.9 ± 1.4 | 12.0 ± 1.1 |
| Striatum | 0.022 ± 0.002 | 0.037 ± 0.005 | 0.031 ± 0.003 |
| Hippocampus | 0.019 ± 0.000 | 0.028 ± 0.006 | 0.024 ± 0.003 |
| Cortex | 0.068 ± 0.015 | 0.078 ± 0.020 | 0.074 ± 0.021 |
| Rest of cerebrum | 0.359 ± 0.086 | 0.580 ± 0.081 | 0.468 ± 0.054 |
| Cerebrum total | 0.446 ± 0.073 | 0.723 ± 0.103 | 0.597 ± 0.062 |
| Cerebellum | 0.170 ± 0.012 | 0.201 ± 0.016 | 0.155 ± 0.024 |
| Blood | 6.5 ± 0.9 | 2.9 ± 0.1 | 3.6 ± 0.2 |
| Carcass | 43.5 ± 1.4 | 61.8 ± 2.1 | 57.8 ± 1.9 |

Data are expressed as mean ± SD;
n = 3 per time point;
^{a}Percentage of ID calculated as cpm in organ/total cpm recovered

In order to correct for differences in body weight between different animals, the % ID/g tissue values were normalized for body weight. The normalized values for striatum, hippocampus, cortex and cerebellum are presented in Table 8.

At 30 min p.i. the radioactivity concentration has increased for all brain regions. This accumulation of radioactivity in all studied brain regions is consistent with the fact that mGluR2 receptors are expressed in several brain areas including hippocampus, cortical regions, olfactory bulb, cerebellum and striatum. Most significant increase was observed for striatum (SUV ˜1.01 at 2 min p.i. to SUV ˜1.70 at 30 min p.i.) The highest radioactivity concentration at 30 min is found in the cerebellum (SUV 2.28), followed by the cortex. For all brain regions the radioactivity concentration at 60 min p.i. is lower compared to 30 min time point, indicating that wash-out has started.

TABLE 8
[¹¹C]B-6 concentration in different brain regions and blood at
2, 30 and 60 min p.i. normalized for the body weight of the animal

| | SUV^{a} | | |
|---|---|---|---|
| Organ | 2 min | 30 min | 60 min |
| Striatum | 1.01 ± 0.01 | 1.70 ± 0.01 | 1.46 ± 0.01 |
| Hippocampus | 0.86 ± 0.01 | 1.36 ± 0.01 | 1.02 ± 0.01 |
| Cortex | 1.04 ± 0.00 | 1.47 ± 0.03 | 1.01 ± 0.01 |
| Cerebrum total | 1.00 ± 0.02 | 1.66 ± 0.02 | 1.24 ± 0.01 |
| Cerebellum | 1.62 ± 0.00 | 2.28 ± 0.01 | 1.57 ± 0.01 |
| Blood | 0.90 ± 0.01 | 0.40 ± 0.00 | 0.50 ± 0.00 |

Data are expressed as mean ± SD;
n = 3 per time point;
^{a}SUV are calculated as (radioactivity in cpm in organ/weight of the organ in g)/(total counts recovered/body weight in g)

### III.e. [¹¹C]B-10

The results of the in vivo distribution study of [¹¹C]B-10 in male Wistar rats is presented in Tables 9 and 10. Table 9 shows the % ID values at 2 min, 30 min and 60 min p.i. of the radiotracer. The total initial brain uptake of the tracer was 0.64% of the ID, with 0.46% ID in the cerebrum and 0.15% ID in the cerebellum. At 2 min p.i. 6.0% of the ID was present in the blood, and this cleared to 3.4% by 60 min p.i. The tracer was cleared mainly by the hepatobiliary system as there was in total 25.5% of ID present in liver and intestines 60 min after injection of the radiotracer. Because of its lipophilic character, the urinary excretion of the tracer was minimal with only 3.0% ID present in the urinary system at 60 min p.i. In view of the large mass of the carcass, significant amount of the ID (˜38% ID at 2 min, ˜63% ID at 30 and 60 min p.i.) was present in the carcass at all time points examined. Typically, carcass constitutes ≧90% of the total body weight of the animal.

TABLE 9
Biodistribution of [¹¹C]B-10 in normal rats at 2, 30 and 60 min p.i.

| | % ID^{a} | | |
|---|---|---|---|
| Organ | 2 min | 30 min | 60 min |
| Urine | 0.1 ± 0.0 | 0.3 ± 0.0 | 0.4 ± 0.1 |
| Kidneys | 7.8 ± 1.1 | 3.3 ± 0.2 | 2.6 ± 0.2 |
| Liver | 32.3 ± 3.2 | 16.2 ± 0.4 | 13.7 ± 1.3 |
| Spleen + Pancreas | 1.5 ± 0.3 | 1.1 ± 0.1 | 1.4 ± 0.5 |
| Lungs | 1.8 ± 0.1 | 0.8 ± 0.0 | 0.7 ± 0.0 |
| Heart | 4.3 ± 0.3 | 1.8 ± 0.1 | 1.2 ± 0.1 |
| Stomach | 1.8 ± 0.1 | 1.8 ± 0.4 | 2.0 ± 0.4 |
| Intestines | 8.5 ± 0.2 | 9.2 ± 1.4 | 11.8 ± 0.0 |
| Striatum | 0.026 ± 0.012 | 0.034 ± 0.005 | 0.035 ± 0.005 |
| Hippocampus | 0.017 ± 0.005 | 0.021 ± 0.004 | 0.026 ± 0.002 |
| Cortex | 0.053 ± 0.025 | 0.071 ± 0.006 | 0.070 ± 0.002 |
| Rest of cerebrum | 0.387 ± 0.084 | 0.511 ± 0.063 | 0.466 ± 0.033 |
| Cerebrum total | 0.456 ± 0.114 | 0.637 ± 0.078 | 0.598 ± 0.036 |
| Cerebellum | 0.149 ± 0.054 | 0.152 ± 0.023 | 0.172 ± 0.030 |
| Blood | 6.0 ± 1.3 | 3.9 ± 0.1 | 3.4 ± 0.2 |
| Carcass | 38.5 ± 2.5 | 62.97 ± 2.4 | 63.7 ± 1.8 |

Data are expressed as mean ± SD;
n = 3 per time point;
^{a}Percentage of ID calculated as cpm in organ/total cpm recovered

In order to correct for differences in body weight between different animals, the % ID/g tissue values were normalized for body weight. The normalized values for striatum, hippocampus, cortex and cerebellum are presented in Table 10.

At 30 min p.i. the radioactivity concentration has increased for almost all brain regions (small decrease for hippocampus but this can be due to an unpunctual dissection of this small brain region). This accumulation of radioactivity in these brain regions is consistent with the fact that mGluR2 receptors are expressed in several brain areas including hippocampus, cortical regions, olfactory bulb, cerebellum and striatum. Most significant increase was observed for cortex (SUV 1.16 at 2 min p.i. to SUV 1.39 at 30 min p.i.). The highest radioactivity concentration at 30 min is found in the cerebellum (SUV 1.68).

TABLE 10
[¹¹C]B-10 concentration in different brain regions and blood at
2, 30 and 60 min p.i. normalized for the body weight of the animal

| | SUV^{a} | | |
|---|---|---|---|
| Organ | 2 min | 30 min | 60 min |
| Striatum | 1.37 ± 0.05 | 1.39 ± 0.03 | 1.55 ± 0.01 |
| Hippocampus | 1.11 ± 0.08 | 0.93 ± 0.02 | 0.94 ± 0.01 |
| Cortex | 1.16 ± 0.04 | 1.39 ± 0.05 | 1.08 ± 0.01 |
| Cerebrum total | 1.12 ± 0.03 | 1.34 ± 0.03 | 1.19 ± 0.01 |
| Cerebellum | 1.59 ± 0.06 | 1.68 ± 0.05 | 1.52 ± 0.02 |
| Blood | 0.90 ± 0.02 | 0.50 ± 0.00 | 0.50 ± 0.00 |

Data are expressed as mean ± SD;
n = 3 per time point;
^{a}SUV are calculated as (radioactivity in cpm in organ/weight of the organ in g)/(total counts recovered/body weight in g)

### III.f [¹¹C]B-3

The results of the in vivo distribution study of [¹¹C]B-3 in male Wistar rats is presented in Tables 11 and 12. Table 11 shows the % ID values at 2 min, 30 min and 60 min p.i. of the radiotracer. At 2 min p.i. 8.5% of the ID was present in the blood, and this cleared to 2.9% by 60 min after injection of the tracer. The total initial brain uptake of the tracer was 0.75%, with 0.54% of the ID in the cerebrum and 0.17% in the cerebellum. At 60 min after injection of the radiotracer, 38.4% ID was present in the liver and intestines. Because of its lipophilic character, the urinary excretion of the tracer was minimal with only 2.8% ID present in the urinary system at 60 min p.i. In view of the large mass of the carcass, significant amount of the ID (˜42%) was present in the carcass at all time points examined Typically, carcass constitutes ≧90% of the total body weight of the animal.

TABLE 11
Biodistribution of [¹¹C]B-3 in normal rats at 2, 30 and 60 min p.i.

| | % ID^{a} | | |
|---|---|---|---|
| Organ | 2 min | 30 min | 60 min |
| Urine | 0.1 ± 0.0 | 0.5 ± 0.1 | 0.4 ± 0.1 |
| Kidneys | 8.8 ± 0.7 | 3.4 ± 1.0 | 2.4 ± 0.9 |
| Liver | 28.7 ± 2.1 | 31.3 ± 9.7 | 23.6 ± 12.9 |
| Spleen + Pancreas | 2.0 ± 0.1 | 1.1 ± 0.3 | 0.9 ± 0.3 |
| Lungs | 3.7 ± 1.7 | 0.5 ± 0.2 | 0.7 ± 0.3 |
| Heart | 4.8 ± 0.4 | 1.7 ± 0.7 | 1.1 ± 0.6 |
| Stomach | 1.5 ± 0.4 | 3.8 ± 1.4 | 9.6 ± 2.2 |
| Intestines | 9.4 ± 0.8 | 8.9 ± 1.4 | 14.8 ± 1.7 |
| Striatum | 0.028 ± 0.002 | 0.027 ± 0.007 | 0.036 ± 0.014 |
| Hippocampus | 0.017 ± 0.002 | 0.019 ± 0.006 | 0.023 ± 0.011 |
| Cortex | 0.062 ± 0.009 | 0.071 ± 0.031 | 0.069 ± 0.027 |
| Rest of cerebrum | 0.457 ± 0.050 | 0.373 ± 0.084 | 0.371 ± 0.119 |
| Cerebrum total | 0.536 ± 0.048 | 0.489 ± 0.121 | 0.499 ± 0.168 |
| Cerebellum | 0.165 ± 0.009 | 0.142 ± 0.042 | 0.142 ± 0.050 |
| Blood | 8.5 ± 1.9 | 2.6 ± 0.7 | 2.9 ± 0.4 |
| Carcass | 36.0 ± 0.7 | 46.7 ± 7.8 | 44.2 ± 10.9 |

Data are expressed as mean ± SD;
n = 3 per time point;
^{a}Percentage of ID calculated as cpm in organ/total cpm recovered

In order to correct for differences in body weight between different animals, the % ID/g tissue values were normalized for body weight. The normalized values for striatum, hippocampus, cortex and cerebellum are presented in Table 12. The radioactivity concentration at 2 and 30 min p.i. is more or less the same in all brain regions. The highest radioactivity concentration is found in the cerebellum (SUV 1.54 at 2 and 30 min p.i.). Accumulation of the radioactivity is observed from 30 to 60 min for all brain regions.

TABLE 12
[¹¹C]B-3 concentration in different brain regions and blood at
2, 30 and 60 min p.i. normalized for the body weight of the animal

| | SUV^{a} | | |
|---|---|---|---|
| Organ | 2 min | 30 min | 60 min |
| Striatum | 0.99 ± 0.00 | 1.15 ± 0.04 | 1.76 ± 0.07 |
| Hippocampus | 0.85 ± 0.01 | 0.84 ± 0.02 | 1.11 ± 0.04 |
| Cortex | 1.03 ± 0.00 | 1.00 ± 0.03 | 1.13 ± 0.04 |
| Cerebrum total | 1.09 ± 0.01 | 1.11 ± 0.03 | 1.38 ± 0.05 |
| Cerebellum | 1.54 ± 0.01 | 1.54 ± 0.04 | 1.84 ± 0.07 |
| Blood | 1.20 ± 0.03 | 0.40 ± 0.01 | 0.40 ± 0.01 |

Data are expressed as mean ± SD;
n = 3 per time point;
^{a}SUV are calculated as (radioactivity in cpm in organ/weight of the organ in g)/(total counts recovered/body weight in g)

The results from these biodistribution studies indicate that although the initial brain uptake is low to modest, there is an accumulation of radioactivity from 2 to 30 min p.i. in all studied brain regions and this is observed for all five ¹¹C-labelled chloropyridinotriazoles [¹¹C]B-4, [¹¹C]B-6, [¹¹C]B-2, [¹¹C]B-7 and [¹¹C]B-10. From 30 to 60 min p.i. wash-out of the radioactivity from brain has started. The tissue distribution looks slightly different for the trifluoromethylpyridinotriazole [¹¹C]B-3. For this tracer the radioactivity concentration at 2 and 30 min p.i. is more or less similar while there is a slight increase from 30 to 60 min p.i. Table 13 gives an overview of the total brain uptake (% ID) at the three studied time points for the six ¹¹C-labelled pyridinotriazoles. [¹¹C]B-2 has the highest total brain uptake at 2 and 30 min p.i. From these biodistribution studies, [¹¹C]B-2 looks the most promising PET tracer for in vivo mGluR2 imaging.

TABLE 13
Comparative total brain uptake in normal rats at 2, 30 and
60 min p.i. for all six studied ¹¹C-labelled tracers

| | Total brain uptake (% ID^{a}) | | |
|---|---|---|---|
| | 2 min p.i. | 30 min p.i. | 60 min p.i. |
| [¹¹C]B-4 | 0.58 ± 0.0 | 0.93 ± 0.2 | 0.76 ± 0.1 |
| [¹¹C]B-2 | 0.88 ± 0.2 | 1.23 ± 0.2 | 0.71 ± 0.0 |
| [¹¹C]B-7 | 0.75 ± 0.1 | 0.93 ± 0.2 | 0.72 ± 0.0 |
| [¹¹C]B-6 | 0.65 ± 0.1 | 0.95 ± 0.1 | 0.76 ± 0.1 |
| [¹¹C]B-10 | 0.64 ± 0.1 | 0.80 ± 0.1 | 0.78 ± 0.0 |
| [¹¹C]B-3 | 0.75 ± 0.0 | 0.64 ± 0.2 | 0.66 ± 0.2 |

Data are expressed as mean ± SD;
n = 3 per time point;
^{a}Percentage of ID calculated as cpm in organ/total cpm recovered

### IV. Plasma Radiometabolite Analysis (30 Min p.i.)

The metabolic stability of [¹¹C]B-4, [¹¹C]B-2, [¹¹C]B-7, and [¹¹C]B-10 was studied in healthy male Wistar rats by determination of the relative amounts of parent tracer and radiometabolites in plasma at 30 min p.i. of the tracer. After intravenous (i.v.) administration of about 74 MBq of the radioligand via tail vein under anesthesia (2.5% Isoflurane in O₂ at 1 L/min flow rate), rats were sacrificed by decapitation at 30 min p.i. (n=2). Blood was collected in heparin containing tubes (4.5 mL LH PST tubes; BD vacutainer, BD, Franklin Lakes, N.J., USA) and stored on ice to stop the metabolism. Next, the blood was centrifuged for 5 min at 3000 rpm to separate the plasma. About 0.5 mL of plasma was spiked with about 10 μg of the authentic non-radioactive compound (1 mg/mL solution) and injected on to HPLC, which was connected to a Chromolith® performance column (C18, 3 mm×100 mm, Merck KGaA, Darmstadt, Germany). The mobile phase consisted of 0.05 M NaOAc buffer (pH 5.5) (solution A) and CH₃CN (solvent B). The following method was used for the analysis: isocratic elution with 100% A for 4 min at a flow rate of 0.5 mL/min, linear gradient to 90% B by 9 min at a flow rate of 1 mL/min, and isocratic elution with mixture of 10% A and 90% B until 12 min. After passing through the UV detector (254 nm), the HPLC eluate was collected as 1 mL fractions (fraction collection each minute) using an automatic fraction collector and the radioactivity of these fractions was measured using an automated gamma counter.

An overview of the results of the plasma radiometabolite analysis for the four studied tracers is presented in Table 14. Of all four studied ¹¹C-labeled tracers, [¹¹C]B-2 is most stable in plasma with 70% of the recovered radioactivity present as the intact tracer 30 min p.i.

TABLE 14
Relative percentages of intact tracer and radiometabolites in rat plasma
at 30 min p.i. of [¹¹C]B-2, [¹¹C]B-4, [¹¹C]B-7, and [¹¹C]B-10

| | Mean ± SD (n = 2) | | | |
|---|---|---|---|---|
| (%) | [¹¹C]B-2 | [¹¹C]B-4 | [¹¹C]B-7 | [¹¹C]B-10 |
| Polar metabolites | 30.3 ± 5.1 | 59.0 ± 7.1 | 69.2 ± 7.0 | 54.5 ± 2.1 |
| Intact tracer | 69.7 ± 5.1 | 41.0 ± 7.1 | 30.8 ± 7.0 | 45.5 ± 2.1 |

Results are presented as mean ± SD (n = 2)

### V. Perfused Brain Radiometabolite Analysis (30 Min p.i.)

The relative amounts of parent tracer and radiometabolites in perfused cerebellum and cerebrum at 30 min p.i. of the tracer was determined in healthy male Wistar rats for [¹¹C]B-4, [¹¹C]B-2, [¹¹C]B-7, and [¹¹C]B-10. After i.v. administration of about 74 MBq of the radioligand via tail vein under anesthesia (2.5% Isoflurane in O₂ at 1 L/min flow rate), rats were sacrificed by administering an overdose of Nembutal (CEVA Santé Animale, 200 mg/kg intraperitoneal). When breathing had stopped, the rats were perfused with saline (Mini Plasco®, Braun, Melsungen, Germany) until the liver turned pale. Brain was isolated, cerebrum and cerebellum were separated and homogenized in 3 mL and 2 mL of CH₃CN, respectively, for about 2 min. A volume of 1 mL of this homogenate was diluted with an equal volume of water and a part of this homogenate was filtered through a 0.22 μm filter (Millipore, Bedford, USA). About 0.5 mL of the filtrate was diluted with 0.1 mL of water and spiked with 10 μg of authentic non-radioactive compound (1 mg/mL solution) for identification of the intact tracer. The cerebrum/cerebellum extract was then injected onto an HPLC system consisting of an analytical XBridge® column (C₁₈, 5 μM, 3 mm×100 mm, Waters) eluted with a mixture of 0.05 M NaOAc buffer (pH 5.5) and CH₃CN (60:40 v/v) at a flow rate of 0.8 mL/min. The HPLC eluate was collected as 1 mL fractions (fraction collection each minute) after passing through the UV detector (254 nm), and the radioactivity in the fractions was measured using an automated gamma counter.

An overview of the results from the perfused rat brain radiometabolite analysis for all four studied tracers is presented in Table 15. Results are very similar for the four studied tracers. The fraction of apolar radiometabolites detected in brain is negligible. The percentage of polar radiometabolites detected in brain is very small. On average, about 90% of the recovered radioactivity was present as intact tracer in both cerebrum as well as in cerebellum for [¹¹C]B-4, [¹¹C]B-2, [¹¹C]B-7, and [¹¹C]B-10.

TABLE 15
Relative percentages of intact tracer and radiometabolites in
perfused rat cerebrum and cerebellum at 30 min
p.i. of [¹¹C]B-4, [¹¹C]B-2, [¹¹C]B-7, and [¹¹C]B-10

| | [¹¹C]B-2 | | [¹¹C]B-4 | | [¹¹C]B-7 | | [¹¹C]B-10 | |
|---|---|---|---|---|---|---|---|---|
| % | cbr | cbll | cbr | cbll | cbr | cbll | cbr | cbll |
| polar | 9.7 ± 0.3 | 4.1 ± 1.5 | 7.6 | 7.3 | 7.1 | 4.5 | 6.9 | 3.6 |
| metab- | | | | | | | | |
| olite | | | | | | | | |
| intact | 90.3 ± 0.3 | 95.5 ± 1.3 | 92.4 | 92.7 | 92.9 | 95.5 | 93.1 | 96.4 |
| tracer | | | | | | | | |

Results are presented as mean ± SD (n = 2) for [¹¹C]B-2.
For all other tracers: n = 1.
cbr = cerebrum,
cbll = cerebellum

### VI. MicroPET (μPET/microPET) Imaging Studies

Imaging experiments were performed on a Focus™ 220 microPET scanner (Concorde Microsystems, Knoxyille, Tenn., USA) using healthy male Wistar rats. During all scan sessions, animals were kept under gas anesthesia (2.5% isoflurane in O₂ at 1 L/min flow rate).

Dynamic scans of 90 min were acquired. After reconstruction of the images (filtered back projection), they were spatially normalized to an in-house created [¹¹C]raclopride template of the rat brain in Paxinos coordinates. Automated and symmetric volumes of interest (VOIs) were generated for different brain regions (striatum, cortex, cerebellum, hippocampus, hypothalamus, thalamus, substantia nigra, nucleus accumbens and lateral globus pallidus) from which time-activity curves (TAC) were constructed for each individual scan, using PMOD software (v 3.1, PMOD Technologies Ltd.). The radioactivity concentration in the different brain regions was expressed as SUV as a function of time p.i. of the radiotracer by normalization for body weight of the animal and injected dose.

Rats were injected with 30-60 MBq of high specific activity formulation of [¹¹C]B-4, [¹¹C]B-2, [¹¹C]B-7, or [¹¹C]B-10 via the tail vein under isoflurane anesthesia (2.5% in O₂ at 1 L/min flow rate).

For pretreatment and displacement experiments, compound A, compound B or ritanserin were dissolved and administered in a vehicle containing 20% (2-hydroxypropyl)-β-cyclodextrine and two equivalents hydrochloric acid. The ritanserin solution was protected from light.

Compound A and compound B have affinity for mGluR2.

A self-blocking study was done by subcutaneous (s.c.) administration of the authentic reference material (for [¹¹C]B-4) at ˜30 min prior to the radiotracer injection. Displacement studies were performed by i.v. injection of compound B at dose 4, 1, 0.3 and 0.1 mg/kg, compound A at dose 1 mg/kg or ritanserin at dose 0.3 mg/kg. All chase compounds were injected ˜30 min after radiotracer injection. A wash-out period of at least four days was maintained between the different pretreatment and displacement studies.

### VI.a. [¹¹C]B-4: Baseline/Self-Blocking/Self-Displacement

[¹¹C]B-4 was evaluated in vivo in three rats which were scanned dynamically for 90 min using μPET. The first rat was used for a baseline scan. The second rat was pretreated with authentic reference material B-4 via s.c. administration (dose 10 mg/kg) at 30 min prior to tracer injection. The third rat was used in a chase experiment and was injected i.v. with authentic reference material B-4 (dose 3 mg/kg) 30 min after tracer injection.

The baseline scan shows uptake of [¹¹C]B-4 in all studied brain regions. Maximum radioactivity concentration is reached after about 9 min p.i. and stays constant until about 27 min p.i., followed by wash-out. Self-blocking results in a lower brain uptake and faster wash-out for all studied brain regions. Injection of the chase results in significant displacement of the radioactivity in all brain areas. These results indicate that [¹¹C]B-4 binds reversible and specific to mGluR2 in striatum, cortex and cerebellum.

VI.b. [¹¹C]B-4, [¹¹C]B-2, [¹¹C]B-7, and [¹¹C]B-10: Baseline/Chase with Compound B

Two rats were injected with high specific activity tracer ([¹¹C]B-4, [¹¹C]B-2, [¹¹C]B-7, or [¹¹C]B-10) and scanned dynamically for 90 min. The first rat was scanned baseline, the second rat was injected i.v. with compound B (dose 4 mg/kg) 30 min after tracer injection. Table 16 gives an overview of the maximum and minimum SUV values in the chase experiment for the four studied tracers.

TABLE 16
Reduction of SUV value (of total brain) due to injection of the chase
compound B (4 mg/kg) for [¹¹C]B-4, [¹¹C]B-2, [¹¹C]B-7,
and [¹¹C]B-10

| SUV | [¹¹C]B-7 | [¹¹C]B-10 | [¹¹C]B-2 | [¹¹C]B-4 |
|---|---|---|---|---|
| before chase | 1.2 | 1.2 | 1.4 | 1.05 |
| after chase | 0.5 | 0.5 | 0.38 | 0.32 |
| % reduction | 58% | 58% | 73% | 70% |

Baseline images showed tracer accumulation in all studied brain regions. After injection of compound B, a structurally unrelated compound with affinity for mGluR2, a significant displacement of the activity was observed for all brain regions, indicating that all four tracers bind reversible and specific to mGluR2. Of the four studied tracers, [¹¹C]B-2 has the highest total brain SUV value before injection of the chase and the lowest total brain SUV value after chase administration. [¹¹C]B-2 shows the strongest displacement (˜73%, largest dynamic range of the four studied tracers), and therefore this tracer was further studied in chase experiments with lower doses of compound B (see section VI.c.).

VI.c. [¹¹C]B-2: Chase with Different Doses of Compound B/Chase with Compound A/Chase with Ritanserin

A chase experiment was performed for [¹¹C]B-2 with different doses of compound B (4, 1, 0.3, 0.1 mg/kg). The chase compound was injected i.v. 30 min after tracer injection. Table 17 gives an overview of the average SUV values before and after injection of the chase for the total brain. This study shows that there is a clear relationship between the administered dose of the chase compound B and the receptor occupancy.

TABLE 17
Reduction of SUV value of [¹¹C]B-2 (of total brain) due to injection of
different doses of chase compound B (4, 1, 0.3, 0.1 mg/kg)

| [¹¹C]B-2 | | Compound B | | | |
|---|---|---|---|---|---|
| SUV | baseline | 4 mg/kg | 1 mg/kg | 0.3 mg/kg | 0.1 mg/kg |
| before chase | 1.26 | 1.36 | 1.78 | 2.15 | 1.35 |
| after chase | | 0.36 | 0.61 | 0.78 | 0.67 |
| % reduction | | 74% | 66% | 64% | 50% |

SUV values are averaged values. Before chase injection: averaged values of time period 930-1650 sec p.i. After chase injection: averaged values of time period 4650-5250 sec p.i.)

To further prove that [¹¹C]B-2 binds selectively to mGluR2, additional chase experiments were performed with compound A, an compound with high selectivity for mGluR2. To exclude binding to the serotonin receptor, an additional chase experiment was performed with ritanserin, a 5HT₂ antagonist.

Compound A displaces the radioligand with a reduction of the average SUV value of about 68% (total brain). Ritanserin has no significant effect on the binding of [¹¹C]B-2. From these chase experiments we can conclude that [¹¹C]B-2 binds reversible, specific and selective to mGluR2.

### VII. Conclusion

Biodistribution studies and baseline microPET imaging in rats showed accumulation of radioactivity in all studied brain regions. Of all six tracers, [¹¹C]B-2 had the highest radioactivity concentration in total brain at 30 min p.i. (>1%) and was most stable in plasma with 70% of the recovered radioactivity present as the intact tracer 30 min p.i. The amount of radiometabolites detected in brain was negligible (<10%). MicroPET chase experiments showed that of all studied tracers [¹¹C]B-2 has the largest dynamic range and binds reversible, specific and selective to mGluR2.

1. A compound according to Formula (I) 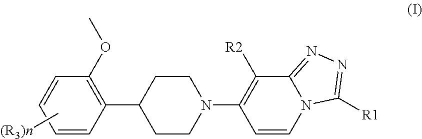
or a stereoisomeric form thereof, wherein
R¹ is selected from the group consisting of cyclopropylmethyl and C₁₋₃alkyl substituted with one or more fluoro substituents;
R² is selected from chloro and trifluoromethyl;
R³ is fluoro;
n is selected from 0, 1 and 2;
wherein at least one C is [¹¹C];
or a salt or thereof.

2. The compound according to claim 1, having the formula [¹¹C]-(I) 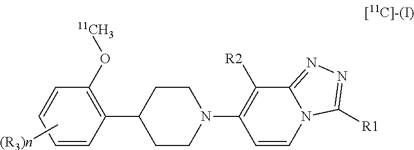
or a stereisomeric form thereof, wherein
R¹ is selected from the group consisting of cyclopropylmethyl and C₁₋₃alkyl substituted with one or more fluoro substituents;
R² is selected from chloro and trifluoromethyl;
R³ is fluoro;
n is selected from 0, 1 and 2;
or a salt thereof.

3. The compound according to claim 1 wherein
R¹ is selected from cyclopropylmethyl and 2,2,2-trifluoroethyl; and
R² is selected from chloro and trifluoromethyl.

4. The compound according to claim 1, wherein R¹ is cyclopropylmethyl and R² is chloro.

5. The compound according to claim 1, wherein n is 0 or 2.

6. The compound according to claim 1, selected from the group consisting of
8-chloro-3-(cyclopropylmethyl)-7-[4-[5-fluoro-2-[¹¹C]methoxyphenyl]-1-piperidinyl]-1,2,4-triazolo[4,3-a]pyridine,
8-chloro-3-(cyclopropylmethyl)-7-[4-[2-fluoro-6-[¹¹C]methoxyphenyl]-1-piperidinyl]-1,2,4-triazolo[4,3-a]pyridine,
8-chloro-7-[4-[5-fluoro-2-[¹¹C]methoxyphenyl]-1-piperidinyl]-3-(2,2,2-trifluoroethyl)-1,2,4-triazolo[4,3-a]pyridine,
8-chloro-7-[4-[2-fluoro-6-[¹¹C]methoxyphenyl]-1-piperidinyl]-3-(2,2,2-trifluoroethyl)-1,2,4-triazolo[4,3-a]pyridine,
8-chloro-3-(cyclopropylmethyl)-7-[4-[2,4-difluoro-6-[¹¹C]methoxyphenyl]-1-piperidinyl]-1,2,4-triazolo[4,3-a]pyridine,
8-chloro-3-(cyclopropylmethyl)-7-[4-(3,6-difluoro-2-[¹¹C]methoxyphenyl)-1-piperidinyl]-1,2,4-triazolo[4,3-a]pyridine,
8-chloro-3-(cyclopropylmethyl)-7-[4-[2,3-difluoro-6-[¹¹C]methoxyphenyl]-1-piperidinyl]-1,2,4-triazolo[4,3-a]pyridine,
8-chloro-3-(cyclopropylmethyl)-7-[4-[3-fluoro-2-[¹¹C]methoxyphenyl]-1-piperidinyl]-1,2,4-triazolo[4,3-a]pyridine,
8-chloro-3-(cyclopropylmethyl)-7-[4-[2-[¹¹C]methoxyphenyl]-1-piperidinyl]-1,2,4-triazolo[4,3-a]pyridine,
8-chloro-3-(cyclopropylmethyl)-7-[4-[3,4-difluoro-2-[¹¹C]methoxyphenyl]-1-piperidinyl]-1,2,4-triazolo[4,3-a]pyridine,
3-(cyclopropylmethyl)-7-[4-[3-fluoro-2-[¹¹C]methoxyphenyl]-1-piperidinyl]-8-(trifluoromethyl)-1,2,4-triazolo[4,3-a]pyridine, and
3-(cyclopropylmethyl)-7-[4-[3,6-difluoro-2-[¹¹C]methoxyphenyl]-1-piperidinyl]-8-(trifluoromethyl)-1,2,4-triazolo[4,3-a]pyridine;
or a stereoisomeric form, or a salt or a solvate thereof.

7. A sterile solution comprising a compound of Formula (I) as defined in claim 1.

8. (canceled)

9. A method of imaging a tissue, cells or a host, comprising contacting with or administering to a tissue, cells or a host, a compound of Formula (I) as defined in claim 1, and imaging the tissue, cells or host with a positron-emission tomography imaging system.

10. A compound according to formula (V) 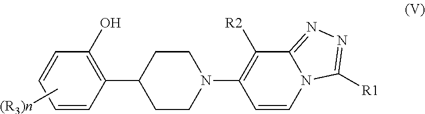
or a stereisomeric form thereof, wherein
R¹ is selected from the group consisting of cyclopropylmethyl and C₁₋₃alkyl substituted with one or more fluoro substituents;
R² is selected from chloro and trifluoromethyl;
R³ is fluoro;
n is selected from 0, 1 and 2;
or a salt thereof;
with the proviso that 2-[1-[8-chloro-3-(cyclopropylmethyl)-1,2,4-triazolo[4,3-a]pyridin-7-yl]-4-piperidinyl]-4-fluoro-phenol is excluded.

11. The compound according to claim 10, wherein n is 0 or 2.

12. The compound according to claim 10, selected from the group consisting of
2-[1-[8-chloro-3-(cyclopropylmethyl)-1,2,4-triazolo[4,3-a]pyridin-7-yl]-4-piperidinyl]-3-fluoro-phenol,
2-[1-[8-chloro-3-(cyclopropylmethyl)-1,2,4-triazolo[4,3-a]pyridin-7-yl]-4-piperidinyl]-3,6-difluoro-phenol,
2-[1-[8-chloro-3-(cyclopropylmethyl)-1,2,4-triazolo[4,3-a]pyridin-7-yl]-4-piperidinyl]-3,5-difluoro-phenol,
2-[1-[8-chloro-3-(cyclopropylmethyl)-1,2,4-triazolo[4,3-a]pyridin-7-yl]-4-piperidinyl]-3,4-difluoro-phenol, and
2-[1-[3-(cyclopropylmethyl)-8-(trifluoromethyl)-1,2,4-triazolo[4,3-a]pyridin-7-yl]-4-piperidinyl]-3,6-difluoro-phenol;
or a stereoisomeric form, or a salt thereof.

13. A process for the preparation of a compound according to Formula [¹¹C]-(I), 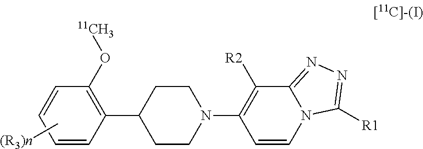
or a stereisomeric form thereof, wherein
R1 is selected from the group consisting of cyclopropylmethyl and C1-3 alkyl substituted with one or more fluoro substituents;
R2 is selected from chloro and trifluoromethyl;
R3 is fluoro;
n is selected from 0, 1 and 2;
or a salt thereof.
comprising the step of reacting a compound according to formula (V) 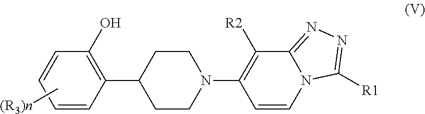
or a stereisomeric form thereof, wherein
R1 is selected from the group consisting of cyclopropylmethyl and C1-3 alkyl substituted with one or more fluoro substituents;
R2 is selected from chloro and trifluoromethyl;
R3 is fluoro;
n is selected from 0, 1 and 2;
or a salt thereof;
with the proviso that 2-[1-[8-chloro-3-(cyclopropylmethyl)-1,2,4-triazolo[4,3-a]pyridin-7-yl]-4-piperidinyl]-4-fluoro-phenol is excluded.
with [¹¹C]CH₃I or [¹¹C]CH₃OTf in the presence of a base in an inert solvent to form [11C]-(I)

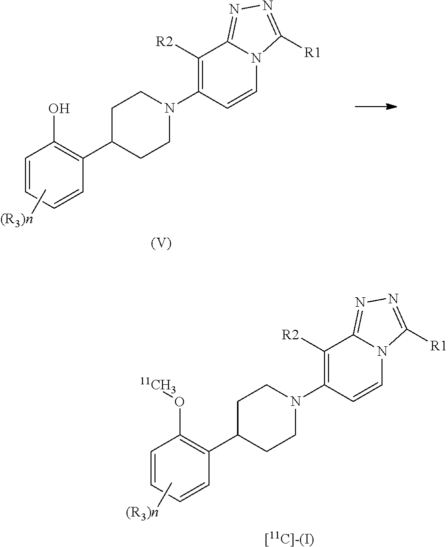

14. A process for the preparation of a compound according to Formula (V), 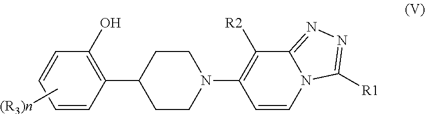
or a stereisomeric form thereof, wherein
R1 is selected from the group consisting of cyclopropylmethyl and C1-3alkyl substituted with one or more fluoro substituents;
R2 is selected from chloro and trifluoromethyl;
R3 is fluoro;
n is selected from 0, 1 and 2;
or a salt thereof;
with the proviso that 2-[1-[8-chloro-3-(cyclopropylmethyl)-1,2,4-triazolo[4,3-a]pyridin-7-yl]-4-piperidinyl]-4-fluoro-phenol is excluded.
comprising
(a) the step of reacting a compound according to formula [¹²C]-(I), with a Lewis acid selected from boron trichloride or boron tribromide in the presence of an inert solvent 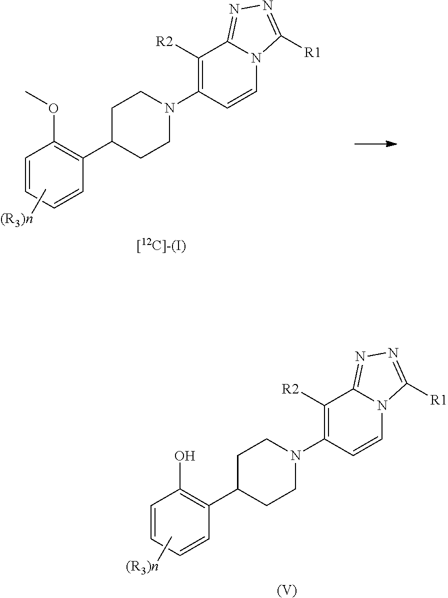
or
(b) the step of reacting a compound according to formula (XX) with a compound of formula (IV), in the presence of a suitable base, in an inert solvent 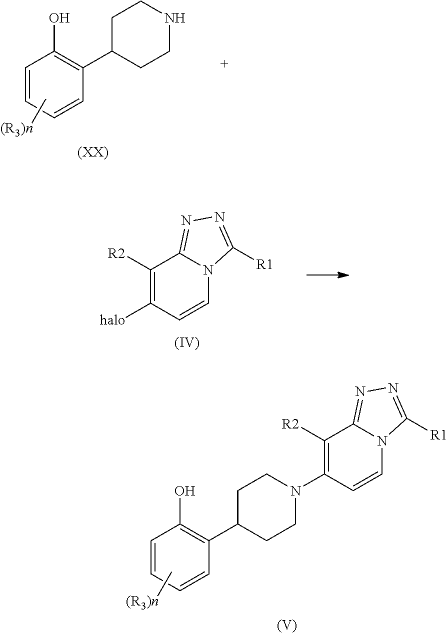
wherein
R¹ is selected from the group consisting of cyclopropylmethyl and C₁₋₃alkyl substituted with one or more fluoro substituents;
R² is selected from chloro and trifluoromethyl;
R³ is fluoro; and
n is selected from 0, 1 and 2.

15. A compound selected from the group consisting of
8-chloro-3-(cyclopropylmethyl)-7-[4-(2,4-difluoro-6-methoxyphenyl)-1-piperidinyl]-1,2,4-triazolo[4,3-a]pyridine,
8-chloro-3-(cyclopropylmethyl)-7-[4-(3,6-difluoro-2-methoxyphenyl)-1-piperidinyl]-1,2,4-triazolo[4,3-a]pyridine,
8-chloro-3-(cyclopropylmethyl)-7-[4-(2,3-difluoro-6-methoxyphenyl)-1-piperidinyl]-1,2,4-triazolo[4,3-a]pyridine,
8-chloro-3-(cyclopropylmethyl)-7-[4-(3-fluoro-2-methoxyphenyl)-1-piperidinyl]-1,2,4-triazolo[4,3-a]pyridine,
8-chloro-3-(cyclopropylmethyl)-7-[4-(2-methoxyphenyl)-1-piperidinyl]-1,2,4-triazolo[4,3-a]pyridine,
8-chloro-3-(cyclopropylmethyl)-7-[4-(3,4-difluoro-2-methoxyphenyl)-1-piperidinyl]-1,2,4-triazolo[4,3-a]pyridine,
3-(cyclopropylmethyl)-7-[4-(3-fluoro-2-methoxyphenyl)-1-piperidinyl]-8-(trifluoromethyl)-1,2,4-triazolo[4,3-a]pyridine, and
3-(cyclopropylmethyl)-7-[4-(3,6-difluoro-2-methoxyphenyl)-1-piperidinyl]-8-(trifluoromethyl)-1,2,4-triazolo[4,3-a]pyridine;
or a stereoisomeric form, or a salt thereof.
